(19) United States
(12) Patent Application Publication
(10) Pub. No.: US 2022/0106296 A1
(43) Pub. Date: Apr. 7, 2022

(54) CYANOTRIAZOLE COMPOUNDS AND USES THEREOF

(71) Applicant: Novartis AG, Basel (CH)

(72) Inventors: Jan Jiricek, Berkeley, CA (US); Shuyi Pearly Ng, Singapore (SG); Srinivasa P S Rao, San Ramon, CA (US)

(21) Appl. No.: 17/253,737

(22) PCT Filed: Jun. 18, 2019
(86) PCT No.: PCT/IB2019/055123

§ 371 Date: Dec. 18, 2020

Related U.S. Application Data
(60) Provisional application No. 62/687,045, filed on Jun. 19, 2018.

(51) Int. Cl. C07D 403/06; C07D 401/14; A61K 45/06

(52) U.S. Cl. CPC C07D 403/06; A61K 45/06; C07D 401/14

(57) ABSTRACT

The present invention provides a compound of Formula (I), or a pharmaceutically acceptable salt thereof: (I) wherein R¹, R², R³, and R⁴ are as defined herein. The present invention further provides therapeutic uses of these compounds, for example against human African typanosomiasis; pharmaceutical compositions comprising these compounds, and compositions comprising these compounds with a therapeutic co-agent.

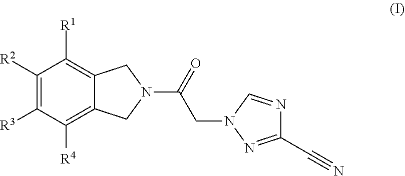

### CROSS-REFERENCE TO RELATED APPLICATIONS

This application claims the benefit of priority to U.S. Ser. No. 62/687,045, filed 19 Jun. 2018, the contents of which is incorporated herein by reference.

### FIELD OF THE INVENTION

The present invention relates to cyanotriazoles compounds, compositions comprising such compounds, and their use for the treatment of kinetoplastid diseases, in particular human African trypanosomiasis (HAT), Chagas disease and leishmaniasis.

### BACKGROUND

Human African Trypanosomiasis (HAT), also known as sleeping sickness, is a parasitic disease caused by the protozoan parasite Trypanosoma brucei (T.b), which is transmitted by the bite of the tsetse fly. Over 95% of sleeping sickness cases reported is caused by T.b. gambiense and <5% of cases are caused by T.b. rhodesiense. The disease is endemic in 36 sub-Saharan African countries and the WHO recently reported that there were <5000 new cases of the disease in 2016, but it is likely that significant numbers of cases remain unreported. Following transmission, HAT manifests itself in 2 stages as the parasite replicates and spreads within the host. Parasites first reside in the bloodstream (stage 1); if untreated, they eventually cross into the central nervous system (CNS) leading to stage 2 disease causing severe neurologic disturbances which eventually cause death.

Four drugs are registered for the treatment of sleeping sickness. The protocol depends on the stage of the disease. The current standard treatment for first-stage disease is intravenous or intramuscular pentamidine (for T.b. gambiense), or intravenous suramin (for T.b. rhodesiense). The current standard treatment for second-stage disease is: Intravenous melarsoprol, or interavenous melarsoprol in combination with oral nifurtimox, intravenous eflornithine only or eflornithine in combination with nifurtimox. All of the drugs have undesirable or sometime serious side effects. For example, 3-10% of patients those injected with Melarsoprol (Arsobal), an organoarsenical, developed reactive encephalopathy (convulsions, progressive coma, or psychotic reactions), and 10-70% of such cases result in death.

The current gold-standard HAT treatment (NECT) combines 7 days eflornithine (2 infusions/day) and 10 days of oral nifurtimox. The administration of this treatment is challenging as it requires an infusion which is extremely complex to administer in resource-poor settings. NECT administration has also led to a significant increase in the cost of HAT treatment, which has led the WHO to question the sustainability of NECT administration in the long term. In particular, it is clear that the current therapies will not be practical for the scaling of disease control and ultimately elimination programs. Hence there is an urgent need for safer, more efficacious and ‘easy to use’ oral drugs for HAT.

The most advanced new molecule is the DNDi clinical candidate fexinidazole, for which stage II trials are ongoing to determine the safety and efficacy of an oral dosing regimen of 1800 mg for 4 days followed by 1200 mg for 6 days. A safety interim analysis of the 300 patients suggests that the compound is at least as effective as the current NECT therapy. Although, the new fexinidazole trial has the potential to successfully replace the infusions required for NECT treatment, the allegedly reported low safety margins and the 10 day long dosing with high pill burden is still a challenging regimen under resource-poor settings. It is thus very important to identify novel short course oral therapy with no need for safety monitoring. We will continuously assess the potential to combine our new drug candidates with fexinidazole (and SCYX-7158) as any oral agents that in combination with these compound could enhance their efficacy and/or improve safety margins could significantly improve the treatment regimen.

Chagas disease, also called American trypanosomiasis, is a tropical parasitic disease caused by the flagellate protozoan Trypanosoma cruzi. T. cruzi is commonly transmitted to humans and other mammals by the blood-sucking “kissing bugs” of the subfamily Triatominae (family Reduviidae).

Chagas disease is contracted primarily in the Americas. It is endemic in twenty one Central and Latin American countries; particularly in poor, rural areas of Mexico, Central America, and South America. Large-scale population movements from rural to urban areas of Latin America and to other regions of the world have increased the geographic distribution of Chagas disease, and cases have been noted in many countries, particularly in Europe. Although there are triatomine bugs in the U.S., only very rarely vectorborne cases of Chagas disease have been documented.

Each year, an estimated 10 to 15 million people across the world are infected with Chagas disease, most of whom do not know they are infected. Every year, 14,000 people die as a consequence of the disease. In Central and South America, Chagas kills more people than any other parasite-borne disease, including malaria. By applying published seroprevalence figures to immigrant populations, CDC estimates that more than 300,000 persons with Trypanosoma cruzi infection live in the United States. Most people with Chagas disease in the United States acquired their infections in endemic countries.

Chagas disease has an acute and a chronic phase. If untreated, infection is lifelong. Acute Chagas disease occurs immediately after infection and may last up to a few weeks or months, and parasites may be found in the circulating blood. Infection may be mild or asymptomatic. There may be fever or swelling around the site of inoculation (where the parasite entered into the skin or mucous membrane). Rarely, acute infection may result in severe inflammation of the heart muscle or the brain and lining around the brain. The initial acute phase is responsive to antiparasitic treatments, with 60-90% cure rates. Following the acute phase, most infected people enter into a prolonged asymptomatic form of disease (called “chronic indeterminate”) during which few or no parasites are found in the blood. During this time, most people are unaware of their infection. Many people may remain asymptomatic for life and never develop Chagas-related symptoms. However, an estimated 20-30% of infected people will develop debilitating and sometimes life-threatening medical problems over the course of their lives.

The symptoms of Chagas disease vary over the course of an infection. In the early, acute stage, symptoms are mild and usually produce no more than local swelling at the site of infection. The initial acute phase is responsive to antiparasitic treatments, with 60-90% cure rates. After 4-8 weeks, individuals with active infections enter the chronic phase of Chagas disease that is asymptomatic for 60-80% of chronically infected individuals through their lifetime.

There is no vaccine against Chagas disease. Treatment for Chagas disease focuses on killing the parasite and managing signs and symptoms.

During the acute phase of Chagas disease, the drugs currently available for treatment are benznidazole and nifurtimox. Once Chagas disease reaches the chronic phase, medications aren't effective for curing the disease. Instead, treatment depends on the specific signs and symptoms. However, problems with these current therapies include their diverse side effects, the length of treatment, and the requirement for medical supervision during treatment.

Leishmaniasis is a disease caused by protozoan parasites that belong to the genus Leishmania and is transmitted by the bite of certain species of sand fly.

Leishmaniasis is mostly a disease of the developing world, and is rarely known in the developed world outside a small number of cases, mostly in instances where troops are stationed away from their home countries. Leishmaniasis can be transmitted in many tropical and subtropical countries, and is found in parts of about 88 countries. Approximately 350 million people live in these areas. The settings in which leishmaniasis is found range from rainforests in Central and South America to deserts in West Asia and the Middle East. It affects as many as 12 million people worldwide, with 1.5-2 million new cases each year. The visceral form of leishmaniasis has an estimated incidence of 500,000 new cases and 60,000 deaths each year. More than 90 percent of the world's cases of visceral leishmaniasis are in India, Bangladesh, Nepal, Sudan, and Brazil. Kabul is estimated as the largest center of cutaneous leishmaniasis in the world, with approximately 67,500 cases as of 2004.

There are four main forms of Leishmaniasis. Cutaneous leishmaniasis is the most common form of leishmaniasis. Visceral leishmaniasis, also called kala-azar, is the most serious form in which the parasites migrate to the vital organs. Visceral leishmaniasis is caused by the parasite Leishmania donovani, and is potentially fatal if untreated. Currently, no vaccines are in routine use.

The two main therapies for visceral leishmaniasis are the antimony derivatives sodium stibogluconate (PENTOSTAM©) and meglumine antimoniate (GLUCANTIM©). Sodium stibogluconate has been used for about 70 years and resistance to this drug is a growing problem. In addition, the treatment is relatively long and painful, and can cause undesirable side effects. Amphotericin (AmBisome) is now the treatment of choice. Miltefosine (Impavido) and paromomycin are the other treatment alternatives. These drugs are known to produce a definitive cure in >90% of patients. Amphotericin (AmBisome) is expansive and has to be given intravenously; it is not affordable to most patients affected. Paromomycin, although affordable, requires intramuscular injections for 3 weeks; compliance is a major issue. Miltefosine is an oral drug and has shown to be more effective and better tolerated than other drugs. However, there are problems associated with the use of miltefosine that arise from its teratogenicity and pharmacokinetics. Miltefosine was shown to be much slower eliminated from the body and was still detectable five months after the end of treatment. The presence of subtherapeutic miltefosine concentrations in the blood beyond five months after treatment might contribute to the selection of resistant parasites and, moreover, the measures for preventing the teratogenic risks of miltefosine must be reconsidered. This led to some reluctance to taking Miltefosine by affected populations.

In view of the foregoing, it is desirable to develop novel compounds as antiparasitic agents. Our invention meets that needs.

Novartis carried out whole cell based high-throughput screening using ˜2 million compounds and obtained ˜28,000 hits showing >50% growth inhibition against T. brucei brucei (Tbb) at 10 μM concentrations. Further biocentric characterization of hits using a battery of biological assays identified the current cyanotriazole series as one of the most potent “concentration and time dependent” cidal inhibitor of T. brucei brucei. This compound series was not only active against T.b. gambiense and T.b. rhodesiense, but also active against T. cruzi, the causative organism of Chagas disease and L. donovani causative agent of visceral leishmaniasis.

### SUMMARY

The present invention relates to a compound of Formula (I):

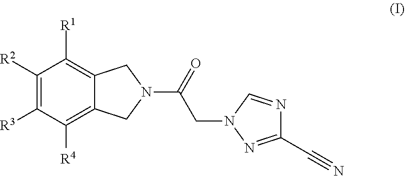

wherein R¹, R², R³, and R⁴ are as defined herein, including stereoisomers, tautomers, pharmaceutically acceptable salts, polymorphs, or solvates thereof, which are useful for the treatment of human African trypanosomiasis.

The present invention also relates to processes and intermediates for making the compounds of the present disclosure.

The present invention also relates to pharmaceutical compositions comprising at least one of the compounds of the present invention and at least one pharmaceutically acceptable carrier, diluent or excipient. The pharmaceutical composition may further comprise at least one additional therapeutic agent. Of particular interest are additional therapeutic agents selected from fexinidazole and SCYX-7158, and combinations thereof.

The compounds of the present invention may be used in the treatment of human African trypanosomiasis.

The compounds of the present invention may be used in therapy.

The compounds of the present invention may be used for the manufacture of a medicament for the treatment of human African trypanosomiasis.

The present invention also relates to a method for the treatment of human African trypanosomiasis, comprising administering to a patient in need thereof a therapeutically effective amount of a first therapeutic agent optionally with a second therapeutic agent, wherein the first therapeutic agent is a compound of the present invention and the second therapeutic agent is one other type of therapeutic agent.

The present invention further relates to a method for the treatment of human African trypanosomiasis, comprising administering to a patient in need thereof a therapeutically effective amount of a compound of the present invention and optionally with a second therapeutic agent that is one other type of therapeutic agent.

The compounds of the present invention can be used alone, in combination with other compounds of the present invention, or in combination with one or more, preferably one to two other agent(s), simultaneously or sequentially.

Other features and advantages of the present invention will be apparent from the following detailed description and claims.

### DETAILED DESCRIPTION

### I. Compounds

In a first embodiment, the present invention provides, inter alia, a compound of Formula (I):

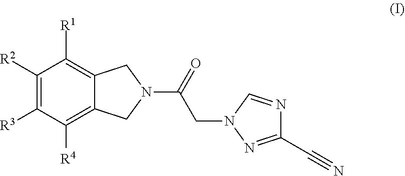

or a pharmaceutically acceptable salt thereof, wherein

R¹, R² and R⁴ are independently H, halogen or C₁-C₄ alkyl;

R³ is independently selected from phenyl and a 5- to 6-membered heteroaryl comprising carbon atoms and 1-4 heteroatoms selected from N, NR^{a}, O, and S(O)ₚ;

wherein said phenyl and heteroaryl are substituted with 0-4 R^{3A};

each R^{3A} is independently selected from halogen, CN, OH, C₁-C₆ alkyl, C₂-C₆ alkenyl, C₂-C₆ alkynyl, C₁-C₆ alkoxy, C₁-C₆ haloalkyl, C₁-C₆ haloalkoxy, C(O)—C₁-C₄ alkyl, and phenyl;

R¹, R² and R⁴ are independently H, halogen or C₁-C₄ alkyl;

each R^{a} is independently selected from H and C₁-C₄ alkyl; and

each p is independently selected from 0, 1 and 2.

In a second embodiment, the present invention includes a compound of any of the embodiments herein, or a pharmaceutically acceptable salt thereof, wherein R³ is phenyl.

In another embodiment, the present invention includes a compound of any of the embodiments herein, or a pharmaceutically acceptable salt thereof, wherein R³ is selected from Ph, 2-F-Ph, 3-F-Ph, 4-F-Ph, 2-Cl-Ph, 3-Cl-Ph, 4-Cl-Ph, 4-Br-Ph, 3-CF₃-Ph, 4-CF₃-Ph, 2-OMe-Ph, 3-OMe-Ph, 4-OMe-Ph, 2-OCF₃-Ph, 4-OCF₃-Ph, 2-CN-Ph, 3-CN-Ph, 4-CN-Ph, 2-C(O)Me-Ph, 1,1′-biphenyl-2-yl, 3,4-diF-Ph, 3,5-diF-Ph, 2-F-4-Cl-Ph, 3-F-4-Cl-Ph, 2-Cl-4-F-Ph, 3-Cl-4-F-Ph, 2,4-diCl-Ph, 2-CF₃-4-F-Ph, 2-CF₃-5-F-Ph, 2-CN-4-F-Ph, 2-F-3-CN-Ph, 2-F-5-CN-Ph, 2-CN-4-F-Ph, 2-OMe-4-F-Ph, 2-OMe-5-F-Ph, 2-Cl-4-OMe-Ph, 2-OMe-5-Cl-Ph, 2-OMe-4-OMe-Ph, 2-OMe-5-OMe-Ph, 3-Me-4-CF₃-Ph, 2-CF₃-4-F-Ph, 2-CF₃-4-OMe-Ph, 2-OMe-5-OCF₃-Ph, 2,4,5-triF-Ph, 2-Cl-4,5-diF-Ph, 2,6-diCl-Ph, 2-CF₃-Ph, 2-Cl-4-CF₃-Ph, 2,4-diF-Ph, and 4-ethynyl-Ph, wherein Ph represents phenyl.

In another embodiment, the present invention includes a compound of any of the embodiments herein, or a pharmaceutically acceptable salt thereof, wherein R³ is pyridinyl.

In another embodiment, the present invention includes a compound of any of the embodiments herein, or a pharmaceutically acceptable salt thereof, wherein R³ is selected from pyrid-4-yl, 2-F-pyrid-3-yl, 6-F-pyrid-3-yl, 2-F-pyrid-4-yl, 3-F-pyrid-4-yl, 2-Cl-pyrid-3-yl, 2-Cl-pyrid-4-yl, 2-CF₃-pyrid-4-yl, 3-Cl-pyrid-4-yl, 3-CF₃-pyrid-4-yl, 2-CF₃-pyrid-3-yl, and 4-CF₃-pyrid-3-yl.

In another embodiment, the present invention includes a compound of any of the embodiments herein, or a pharmaceutically acceptable salt thereof, wherein at least one of R¹, R² or R³ is H.

In another embodiment, the present invention includes a compound of any of the embodiments herein, wherein the compound is of Formula IA:

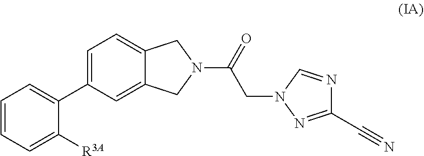

or a pharmaceutically acceptable salt thereof.

In another embodiment, the present invention includes a compound of any of the embodiments herein, wherein the compound is of Formula (IB):

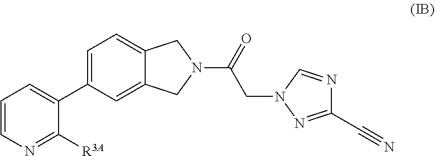

or a pharmaceutically acceptable salt thereof.

In another embodiment, the present invention includes a compound of any of the embodiments herein, wherein the compound is of Formula (IC):

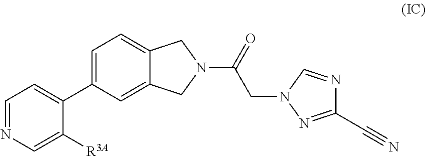

or a pharmaceutically acceptable salt thereof.

In another embodiment, the present invention includes a compound of any of the embodiments herein, or a pharmaceutically acceptable salt thereof, wherein R^{3A} is independently selected from -Me, —OH, —F, —Cl, —CH₂F, —CHF₂, —CF₃, —CH₂CF₃, —OMe, —OCF₃ and —O—CH₂—CF₃.

In another embodiment, the compound of the present invention or a pharmaceutically acceptable salt thereof, is selected from 1-(2-(5-(2-(difluoromethyl)pyridin-3-yl)isoindolin-2-yl)-2-oxoethyl)-1H-1,2,4-triazole-3-carbonitrile; 1-(2-(5-(3-chloro-5-(trifluoromethyl)pyridin-4-yl)isoindolin-2-yl)-2-oxoethyl)-1H-1,2,4-triazole-3-carbonitrile; 1-(2-(5-(5-chloro-2-fluoropyridin-4-yl)isoindolin-2-yl)-2-oxoethyl)-1H-1,2,4-triazole-3-carbonitrile; 1-(2-(5-(3-chloro-2-(trifluoromethyl)phenyl)isoindolin-2-yl)-2-oxoethyl)-1H-1,2,4-triazole-3-carbonitrile; 1-(2-oxo-2-(5-(2-(2,2,2-trifluoroethoxy)pyridin-3-yl)isoindolin-2-yl)ethyl)-1H-1,2,4-triazole-3-carbonitrile; 1-(2-(5-(6-methyl-2-(trifluoromethyl)pyridin-3-yl)isoindolin-2-yl)-2-oxoethyl)-1H-1,2,4-triazole-3-carbonitrile; 1-(2-(5-(5-fluoro-2-(trifluoromethyl)pyridin-3-yl)isoindolin-2-yl)-2-oxoethyl)-1H-1,2,4-triazole-3-carbonitrile; 1-(2-(5-(5-fluoro-4-(trifluoromethyl)pyridin-3-yl)isoindolin-2-yl)-2-oxoethyl)-1H-1,2,4-triazole-3-carbonitrile; 1-(2-(5-(2-chloro-4,6-difluorophenyl)isoindolin-2-yl)-2-oxoethyl)-1H-1,2,4-triazole-3-carbonitrile; 1-(2-(5-(4-fluoro-2-(pyrrolidin-1-yl)phenyl)isoindolin-2-yl)-2-oxoethyl)-1H-1,2,4-triazole-3-carbonitrile; 1-(2-(5-(2-fluoro-6-(trifluoromethyl)phenyl)isoindolin-2-yl)-2-oxoethyl)-1H-1,2,4-triazole-3-carbonitrile; 1-(2-(5-(2-methylpyridin-3-yl)isoindolin-2-yl)-2-oxoethyl)-1H-1,2,4-triazole-3-carbonitrile; 1-(2-(5-(6-methoxy-2-(trifluoromethyl)pyridin-3-yl)isoindolin-2-yl)-2-oxoethyl)-1H-1,2,4-triazole-3-carbonitrile; 1-(2-(5-(2-chloro-3,6-difluorophenyl)isoindolin-2-yl)-2-oxoethyl)-1H-1,2,4-triazole-3-carbonitrile; 1-(2-(5-(3-fluoro-5-(trifluoromethyl)pyridin-4-yl)isoindolin-2-yl)-2-oxoethyl)-1H-1,2,4-triazole-3-carbonitrile; 1-(2-oxo-2-(5-(2-(trifluoromethoxy)pyridin-3-yl)isoindolin-2-yl)ethyl)-1H-1,2,4-triazole-3-carbonitrile; 1-(2-(5-(4-chloro-2-(trifluoromethyl)pyridin-3-yl)isoindolin-2-yl)-2-oxoethyl)-1H-1,2,4-triazole-3-carbonitrile; 1-(2-oxo-2-(5-(2-(2,2,2-trifluoroethyl)pyridin-3-yl)isoindolin-2-yl)ethyl)-1H-1,2,4-triazole-3-carbonitrile; 1-(2-(5-(4-fluoro-2-(trifluoromethyl)pyridin-3-yl)isoindolin-2-yl)-2-oxoethyl)-1H-1,2,4-triazole-3-carbonitrile; 1-(2-(5-(2-chloro-4-fluoropyridin-3-yl)isoindolin-2-yl)-2-oxoethyl)-1H-1,2,4-triazole-3-carbonitrile; 1-(2-(5-(2-fluoro-4-(trifluoromethyl)pyridin-3-yl)isoindolin-2-yl)-2-oxoethyl)-1H-1,2,4-triazole-3-carbonitrile; 1-(2-(5-(3-chloro-5-fluoropyridin-4-yl)isoindolin-2-yl)-2-oxoethyl)-1H-1,2,4-triazole-3-carbonitrile; 1-(2-(5-(4-chloro-2-fluoropyridin-3-yl)isoindolin-2-yl)-2-oxoethyl)-1H-1,2,4-triazole-3-carbonitrile; 1-(2-(5-(1-methyl-3-(trifluoromethyl)-1H-pyrazol-4-yl)isoindolin-2-yl)-2-oxoethyl)-1H-1,2,4-triazole-3-carbonitrile; 1-(2-(5-(6-fluoro-2-(trifluoromethyl)pyridin-3-yl)isoindolin-2-yl)-2-oxoethyl)-1H-1,2,4-triazole-3-carbonitrile; 1-(2-oxo-2-(5-(3-(trifluoromethyl)pyridin-4-yl)isoindolin-2-yl)ethyl)-1H-1,2,4-triazole-3-carbonitrile; 1-(2-(5-(5-fluoro-2-(trifluoromethyl)phenyl)isoindolin-2-yl)-2-oxoethyl)-1H-1,2,4-triazole-3-carbonitrile; 1-(2-(5-(4-fluoro-2-(trifluoromethyl)phenyl)isoindolin-2-yl)-2-oxoethyl)-1H-1,2,4-triazole-3-carbonitrile; 1-(2-(5-(2-chloro-4,5-difluorophenyl)isoindolin-2-yl)-2-oxoethyl)-1H-1,2,4-triazole-3-carbonitrile; 1-(2-(5-(4-methoxy-2-(trifluoromethyl)phenyl)isoindolin-2-yl)-2-oxoethyl)-1H-1,2,4-triazole-3-carbonitrile; 1-(2-(5-(2-cyano-4-fluorophenyl)isoindolin-2-yl)-2-oxoethyl)-1H-1,2,4-triazole-3-carbonitrile; 1-(2-(5-(2-chlorophenyl)isoindolin-2-yl)-2-oxoethyl)-1H-1,2,4-triazole-3-carbonitrile; 1-(2-(5-(2-chloro-4-methoxyphenyl)isoindolin-2-yl)-2-oxoethyl)-1H-1,2,4-triazole-3-carbonitrile; 1-(2-(5-(2,6-dichlorophenyl)isoindolin-2-yl)-2-oxoethyl)-1H-1,2,4-triazole-3-carbonitrile; 1-(2-oxo-2-(5-(2-(trifluoromethyl)phenyl)isoindolin-2-yl)ethyl)-1H-1,2,4-triazole-3-carbonitrile; 1-(2-(5-(2-chloro-4-fluorophenyl)isoindolin-2-yl)-2-oxoethyl)-1H-1,2,4-triazole-3-carbonitrile; 1-(2-(5-(2,4-dichlorophenyl)isoindolin-2-yl)-2-oxoethyl)-1H-1,2,4-triazole-3-carbonitrile, 1-(2-(5-(4-fluoro-2-(2-methoxyethoxy)phenyl)isoindolin-2-yl)-2-oxoethyl)-1H-1,2,4-triazole-3-carbonitrile; 1-(2-oxo-2-(5-(2-(trifluoromethoxy)phenyl)isoindolin-2-yl)ethyl)-1H-1,2,4-triazole-3-carbonitrile; 1-(2-(5-(5-cyano-2-fluorophenyl)isoindolin-2-yl)-2-oxoethyl)-1H-1,2,4-triazole-3-carbonitrile; 1-(2-(5-(2-chloro-4-(trifluoromethyl)phenyl)isoindolin-2-yl)-2-oxoethyl)-1H-1,2,4-triazole-3-carbonitrile; 1-(2-(5-(2-cyanophenyl)isoindolin-2-yl)-2-oxoethyl)-1H-1,2,4-triazole-3-carbonitrile; 1-(2-(5-(3-cyano-2-fluorophenyl)isoindolin-2-yl)-2-oxoethyl)-1H-1,2,4-triazole-3-carbonitrile; 1-(2-oxo-2-(5-(2,4,5-trifluorophenyl)isoindolin-2-yl)ethyl)-1H-1,2,4-triazole-3-carbonitrile; 1-(2-(5-(4-cyanophenyl)isoindolin-2-yl)-2-oxoethyl)-1H-1,2,4-triazole-3-carbonitrile; 1-(2-(5-(2-chloropyridin-3-yl)isoindolin-2-yl)-2-oxoethyl)-1H-1,2,4-triazole-3-carbonitrile; 1-(2-(5-(1-methyl-3-(trifluoromethyl)-1H-pyrazol-5-yl)isoindolin-2-yl)-2-oxoethyl)-1H-1,2,4-triazole-3-carbonitrile; 1-(2-(5-(3-chloropyridin-4-yl)isoindolin-2-yl)-2-oxoethyl)-1H-1,2,4-triazole-3-carbonitrile; 1-(2-(5-(2-acetylphenyl)isoindolin-2-yl)-2-oxoethyl)-1H-1,2,4-triazole-3-carbonitrile; 1-(2-(5-(4-fluoro-2-methoxyphenyl)isoindolin-2-yl)-2-oxoethyl)-1H-1,2,4-triazole-3-carbonitrile; 1-(2-(5-(5-chloro-2-methoxyphenyl)isoindolin-2-yl)-2-oxoethyl)-1H-1,2,4-triazole-3-carbonitrile; 1-(2-(5-(2,4-dimethoxyphenyl)isoindolin-2-yl)-2-oxoethyl)-1H-1,2,4-triazole-3-carbonitrile; 1-(2-(5-(3-cyanophenyl)isoindolin-2-yl)-2-oxoethyl)-1H-1,2,4-triazole-3-carbonitrile; 1-(2-(5-(2,4-difluorophenyl)isoindolin-2-yl)-2-oxoethyl)-1H-1,2,4-triazole-3-carbonitrile; 1-(2-(5-(6-fluoropyridin-3-yl)isoindolin-2-yl)-2-oxoethyl)-1H-1,2,4-triazole-3-carbonitrile; 1-(2-(5-(5-fluoro-2-methoxyphenyl)isoindolin-2-yl)-2-oxoethyl)-1H-1,2,4-triazole-3-carbonitrile; 1-(2-(5-(4-chloro-2-fluorophenyl)isoindolin-2-yl)-2-oxoethyl)-1H-1,2,4-triazole-3-carbonitrile; 1-(2-(5-(2,5-dimethoxyphenyl)isoindolin-2-yl)-2-oxoethyl)-1H-1,2,4-triazole-3-carbonitrile; 1-(2-(5-(3-fluoropyridin-4-yl)isoindolin-2-yl)-2-oxoethyl)-1H-1,2,4-triazole-3-carbonitrile; 1-(2-(5-(4-methoxyphenyl)isoindolin-2-yl)-2-oxoethyl)-1H-1,2,4-triazole-3-carbonitrile; 1-(2-(5-(2-methoxy-5-(trifluoromethoxy)phenyl)isoindolin-2-yl)-2-oxoethyl)-1H-1,2,4-triazole-3-carbonitrile; 1-(2-(5-(2-fluoropyridin-4-yl)isoindolin-2-yl)-2-oxoethyl)-1H-1,2,4-triazole-3-carbonitrile; 1-(2-(5-(4-ethynylphenyl)isoindolin-2-yl)-2-oxoethyl)-1H-1,2,4-triazole-3-carbonitrile; 1-(2-(5-(2-fluorophenyl)isoindolin-2-yl)-2-oxoethyl)-1H-1,2,4-triazole-3-carbonitrile; 1-(2-oxo-2-(5-(pyridin-4-yl)isoindolin-2-yl)ethyl)-1H-1,2,4-triazole-3-carbonitrile; 1-(2-(5-(4-bromophenyl)isoindolin-2-yl)-2-oxoethyl)-1H-1,2,4-triazole-3-carbonitrile; 1-(2-(5-(3-chloro-4-fluorophenyl)isoindolin-2-yl)-2-oxoethyl)-1H-1,2,4-triazole-3-carbonitrile; 1-(2-(5-([1,1′-biphenyl]-2-yl)isoindolin-2-yl)-2-oxoethyl)-1H-1,2,4-triazole-3-carbonitrile; 1-(2-(5-(4-chloro-3-fluorophenyl)isoindolin-2-yl)-2-oxoethyl)-1H-1,2,4-triazole-3-carbonitrile; 1-(2-(5-(4-chlorophenyl)isoindolin-2-yl)-2-oxoethyl)-1H-1,2,4-triazole-3-carbonitrile; 1-(2-(5-(3,4-difluorophenyl)isoindolin-2-yl)-2-oxoethyl)-1H-1,2,4-triazole-3-carbonitrile; 1-(2-(5-(2-fluoropyridin-3-yl)isoindolin-2-yl)-2-oxoethyl)-1H-1,2,4-triazole-3-carbonitrile; 1-(2-(5-(3,5-difluorophenyl)isoindolin-2-yl)-2-oxoethyl)-1H-1,2,4-triazole-3-carbonitrile; 1-(2-(5-(2-methoxyphenyl)isoindolin-2-yl)-2-oxoethyl)-1H-1,2,4-triazole-3-carbonitrile; 1-(2-oxo-2-(5-(4-(trifluoromethyl)phenyl)isoindolin-2-yl)ethyl)-1H-1,2,4-triazole-3-carbonitrile; 1-(2-(5-(3-methoxyphenyl)isoindolin-2-yl)-2-oxoethyl)-1H-1,2,4-triazole-3-carbonitrile; 1-(2-(5-(3-methyl-4-(trifluoromethyl)phenyl)isoindolin-2-yl)-2-oxoethyl)-1H-1,2,4-triazole-3-carbonitrile; 1-(2-oxo-2-(5-phenylisoindolin-2-yl)ethyl)-1H-1,2,4-triazole-3-carbonitrile; 1-(2-(5-(3-chlorophenyl)isoindolin-2-yl)-2-oxoethyl)-1H-1,2,4-triazole-3-carbonitrile; 1-(2-(5-(3-fluorophenyl)isoindolin-2-yl)-2-oxoethyl)-1H-1,2,4-triazole-3-carbonitrile; 1-(2-(5-(4-fluorophenyl)isoindolin-2-yl)-2-oxoethyl)-1H-1,2,4-triazole-3-carbonitrile; 1-(2-oxo-2-(5-(4-(trifluoromethoxy)phenyl)isoindolin-2-yl)ethyl)-1H-1,2,4-triazole-3-carbonitrile; 1-(2-(5-(3-fluoro-2-(trifluoromethyl)phenyl)isoindolin-2-yl)-2-oxoethyl)-1H-1,2,4-triazole-3-carbonitrile; 1-(2-oxo-2-(5-(2-(trifluoromethyl)pyridin-3-yl)isoindolin-2-yl)ethyl)-1H-1,2,4-triazole-3-carbonitrile; 1-(2-oxo-2-(5-(4-(trifluoromethyl)pyridin-3-yl)isoindolin-2-yl)ethyl)-1H-1,2,4-triazole-3-carbonitrile.

The invention provides a novel class of compounds, pharmaceutical compositions comprising such compounds and methods of using such compounds to treat or prevent diseases or disorders associated with a parasite. In particular, the compounds can be used to treat leishmaniasis, Human Trypanosomiasis and/or Chagas disease. The compounds of the invention are effective in inhibiting, ameliorating, or eradicating the pathology and/or symptomology of the parasite.

In another embodiment, the compounds of the present invention exhibit IC₅₀ values ≤10 μM, using the growth inhibition assays disclosed herein, preferably, IC₅₀ values ≤5 μM, more preferably, IC₅₀ values ≤1.0 μM, even more preferably, IC₅₀ values ≤0.5 μM.

### II. Other Embodiments

In another embodiment, the present invention provides a composition comprising at least one of the compounds of the present invention or a pharmaceutically acceptable salt thereof.

In another embodiment, the present invention provides a pharmaceutical composition comprising at least one of the compounds of the present invention or a pharmaceutically acceptable salt thereof and at least one pharmaceutically acceptable carrier, diluent or excipient.

In another embodiment, the present invention provides a pharmaceutical composition, comprising a therapeutically effective amount of at least one of the compounds of the present invention or a pharmaceutically acceptable salt thereof and at least one pharmaceutically acceptable carrier, diluent or excipient.

The pharmaceutical composition is useful in the treatment or prevention of diseases or disorders associated with a parasite.

The pharmaceutical composition is useful in the treatment or prevention of leishmaniasis.

The pharmaceutical composition is useful in the treatment or prevention of human African trypanosomiasis.

The pharmaceutical composition is useful in the treatment or prevention of Chagas disease.

In another embodiment, the present invention provides a pharmaceutical composition as defined above further comprising additional therapeutic agent(s).

In another embodiment, the present invention provides a process for making a compound of the present invention.

In another embodiment, the present invention provides an intermediate for making a compound of the present invention.

In another embodiment, the present invention provides a compound of the present invention, for use in therapy, alone, or optionally in combination with another compound of the present invention and/or at least one other type of therapeutic agent.

In another embodiment, the present invention provides a compound of the present invention for use in therapy, for the treatment of leishmaniasis, human African trypanosomiasis, or Chagas disease, alone, or optionally in combination with another compound of the present invention and/or at least one other type of therapeutic agent.

In another embodiment, the present invention provides a method for the treatment of leishmaniasis, human African trypanosomiasis, or Chagas disease, comprising administering to a patient in need of such treatment a therapeutically effective amount of at least one of the compounds of the present invention, alone, or optionally in combination with another compound of the present invention and/or at least one other type of therapeutic agent.

In another embodiment, the present invention provides a method for the treatment of leishmaniasis, human African trypanosomiasis, or Chagas disease, comprising administering to a patient in need thereof a therapeutically effective amount of a first and second therapeutic agent, wherein the first therapeutic agent is a compound of the present invention and the second therapeutic agent is one other type of therapeutic agent.

In another embodiment, the present invention also provides the use of a compound of the present invention for the manufacture of a medicament for the treatment of leishmaniasis, human African trypanosomiasis, or Chagas disease, alone, or optionally in combination with another compound of the present invention and/or at least one other type of therapeutic agent.

In another embodiment, the present invention provides a combined preparation of a compound of the present invention and additional therapeutic agent(s) for simultaneous, separate or sequential use in therapy.

In another embodiment, the present invention provides a combined preparation of a compound of the present invention and additional therapeutic agent(s) for simultaneous, separate or sequential use in the treatment of leishmaniasis, human African trypanosomiasis, or Chagas disease. The compound may be administered as a pharmaceutical composition described herein.

In another embodiment, the present invention provides a method for the treatment of leishmaniasis, human African trypanosomiasis, or Chagas disease, comprising administering to a patient in need thereof a therapeutically effective amount of a compound of the present invention and optionally with a second therapeutic agent that is one other type of therapeutic agent.

In another embodiment, additional therapeutic agent(s) used in combined pharmaceutical compositions or combined methods or combined uses, are selected from one or more, preferably one to three, of the following therapeutic agents: For treating leishmaniasis, meglumine antimoniate, stibogluconate, Amphotericin, Miltefosine and paromomycin; for treating human African trypanosomiasis, pentamidine, suramin, melarsoprol, eflornithine, fexinidazole and SCYX-7158; and for treating Chagas disease, benznidazole, nifurtimox and Amphotericin b.

Various (enumerated) embodiments of the invention are described herein. It will be recognized that features specified in each embodiment may be combined with other specified features to provide further embodiments of the present invention. It is also understood that each individual element of the embodiments is its own independent embodiment.

Other features of the present invention should become apparent in the course of the above descriptions of exemplary embodiments that are given for illustration of the invention and are not intended to be limiting thereof.

### III. Definitions

The general terms used hereinbefore and hereinafter preferably have within the context of this invention the following meanings, unless otherwise indicated, where more general terms wherever used may, independently of each other, be replaced by more specific definitions or remain, thus defining more detailed embodiments of the invention.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

The term “a,” “an,” “the” and similar terms used in the context of the present invention (especially in the context of the claims) are to be construed to cover both the singular and plural unless otherwise indicated herein or clearly contradicted by the context.

As used herein, the term “heteroatoms” refers to nitrogen (N), oxygen (O) or sulfur (S) atoms, in particular nitrogen or oxygen. Unless otherwise indicated, any heteroatom with unsatisfied valences is assumed to have hydrogen atoms sufficient to satisfy the valences.

As used herein, the terms “alkyl” refers to a hydrocarbon radical of the general formula CₙH₂ₙ₊₁. The alkane radical may be straight or branched. For example, the term “C₁-C₁₀ alkyl” or “C₁ to C₁₀ alkyl” refers to a monovalent, straight, or branched aliphatic group containing 1 to 10 carbon atoms (e.g., methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, heptyl, and the like).

The term “alkylene” refers to a divalent alkyl group. For example, the term “C₁-C₆ alkylene” or “C₁ to C₆ alkylene” refers to a divalent, straight, or branched aliphatic group containing 1 to 6 carbon atoms (e.g., methylene (—CH₂—), ethylene (—CH₂CH₂—), n-propylene (—CH₂CH₂CH₂—), iso-propylene (—CH(CH₃)CH₂—), n-butylene, sec-butylene, iso-butylene, tert-butylene, n-pentylene, isopentylene, neopentylene, n-hexylene and the like).

The term “alkoxy” refers to an alkyl linked to an oxygen, which may also be represented as —O—R or —OR, wherein the R represents the alkyl group. “C₁-C₆ alkoxy” or “C₁ to C₆ alkoxy” is intended to include C₁, C₂, C₃, C₄, C₅, and C₆ alkoxy groups. Example alkoxy groups include, but are not limited to, methoxy, ethoxy, propoxy (e.g., n-propoxy and isopropoxy), and t-butoxy. Similarly, “alkylthio” or “thioalkoxy” represents an alkyl group as defined above with the indicated number of carbon atoms attached through a sulphur bridge; for example methyl-S— and ethyl-S—.

“Halogen” or “halo” may be fluorine, chlorine, bromine or iodine (preferred halogens as substituents are fluorine and chlorine).

“Haloalkyl” is intended to include both branched and straight-chain saturated aliphatic hydrocarbon groups having the specified number of carbon atoms, substituted with one or more halogens. Examples of haloalkyl include, but are not limited to, fluoromethyl, difluoromethyl, trifluoromethyl, trichloromethyl, pentafluoroethyl, pentachloroethyl, 2,2,2-trifluoroethyl, heptafluoropropyl, and heptachloropropyl. Examples of haloalkyl also include “fluoroalkyl” that is intended to include both branched and straight-chain saturated aliphatic hydrocarbon groups having the specified number of carbon atoms, substituted with one or more fluorine atoms.

“Haloalkoxy” represents a haloalkyl group as defined above with the indicated number of carbon atoms attached through an oxygen bridge. For example, “C₁-C₆ haloalkoxy” or “C₁ to C₆ haloalkoxy” is intended to include C₁, C₂, C₃, C₄, C₅, and C₆ haloalkoxy groups. Examples of haloalkoxy include, but are not limited to, trifluoromethoxy, 2,2,2-trifluoroethoxy, and pentafluorothoxy. Similarly, “haloalkylthio” or “thiohaloalkoxy” represents a haloalkyl group as defined above with the indicated number of carbon atoms attached through a sulphur bridge; for example trifluoromethyl-S—, and pentafluoroethyl-S—.

The term “oxo” or —C(O)— refers to a carbonyl group. For example, a ketone, aldehyde, or part of an acid, ester, amide, lactone, or lactam group.

The term “cycloalkyl” refers to nonaromatic carbocyclic ring that is fully hydrogenated ring, including mono-, bi- or poly-cyclic ring systems. “C₃-C₈ cycloalkyl” or “C₃ to C₈ cycloalkyl” is intended to include C₃, C₄, C₅, C₆, C₇ and C₈ cycloalkyl groups. Example cycloalkyl groups include, but are not limited to, cyclopropyl, cyclobutyl, cyclopentyl, cyclohexyl, and norbornyl.

The term “aryl” refers to 6- to 10-membered aromatic carbocyclic moieties having a single (e.g., phenyl) or a fused ring system (e.g., naphthalene.). A typical aryl group is phenyl group.

The term “benzyl”, as used herein, refers to a methyl group on which one of the hydrogen atoms is replaced by a phenyl group.

“Heterocycloalkyl” means cycloalkyl, as defined in this application, provided that one or more of the ring carbons indicated, are replaced by a moiety selected from —O—, —N═, —NR—, —C(O)—, —S—, —S(O)— and —S(O)₂—, wherein R is hydrogen, C₁₋₄alkyl or a nitrogen protecting group (for example, carbobenzyloxy, p-methoxybenzyl carbonyl, t-butoxycarbonyl, acetyl, benzoyl, benzyl, p-methoxy-benzyl, p-methoxy-phenyl, 3,4-dimethoxybenzyl, and the like). For example, a 3 to 8 membered heterocycloalkyl includes epoxy, aziridinyl, azetidinyl, imidazolidinyl, pyrazolidinyl, tetrahydrofuranyl, tetrahydrothienyl, tetrahydrothienyl 1,1-dioxide, oxazolidinyl, thiazolidinyl, pyrrolidinyl, pyrrolidinyl-2-one, morpholino, piperazinyl, piperidinyl, piperidinylone, pyrazolidinyl, hexahydropyrimidinyl, 1,4-dioxa-8-aza-spiro[4.5]dec-8-yl, thiomorpholino, sulfanomorpholino, sulfonomorpholino, octahydropyrrolo[3,2-b]pyrrolyl, and the like.

The term “partially saturated heterocycle” refers to a nonaromatic ring that is partially hydrogenated and may exist as a single ring, bicyclic ring (including fused rings). Unless specified otherwise, said heterocyclic ring is generally a 5- to 10-membered ring containing 1 to 3 heteroatoms selected from —O—, —N═, —NR—, and —S—, (preferably 1 or 2 heteroatoms). Partially saturated heterocyclic rings include groups such as dihydrofuranyl, dihydrooxazolyl, dihydropyridinyl, imidazolinyl, 1H-dihydroimidazolyl, 2H-pyranyl, 4H-pyranyl, 2H-chromenyl, oxazinyl and the like. A partially saturated heterocyclic ring also includes groups wherein the heterocyclic ring is fused to an aryl or heteroaryl ring (e.g., 2,3-dihydrobenzofuranyl, indolinyl (or 2,3-dihydroindolyl), 2,3-dihydrobenzothiophenyl, 2,3-dihydrobenzothiazolyl, 1,2,3,4-tetrahydroquinolinyl, 1,2,3,4-tetrahydroisoquinolinyl, 5,6,7,8-tetrahydropyrido[3,4-b]pyrazinyl, and the like).

The term “partially or fully saturated heterocycle” refers to a nonaromatic ring that is either partially or fully hydrogenated and may exist as a single ring, bicyclic ring (including fused rings) or a spiral ring. Unless specified otherwise, the heterocyclic ring is generally a 3- to 12-membered ring containing 1 to 3 heteroatoms (preferably 1 or 2 heteroatoms) independently selected from sulfur, oxygen and/or nitrogen. When the term “partially or fully saturated heterocycle” is used, it is intended to include “heterocycloalkyl”, and “partially saturated heterocycle”. Examples of spiral rings include 2,6-diazaspiro[3.3]heptanyl, 3-azaspiro[5.5]undecanyl, 3,9-diazaspiro[5.5]undecanyl, and the like.

The term “heteroaryl” refers to aromatic moieties containing at least one heteroatom (e.g., oxygen, sulfur, nitrogen or combinations thereof) within a 5- to 10-membered aromatic ring system (e.g., pyrrolyl, pyridyl, pyrazolyl, indolyl, indazolyl, thienyl, furanyl, benzofuranyl, oxazolyl, isoxazolyl, imidazolyl, triazolyl, tetrazolyl, triazinyl, pyrimidinyl, pyrazinyl, thiazolyl, purinyl, benzimidazolyl, quinolinyl, isoquinolinyl, quinoxalinyl, benzopyranyl, benzothiophenyl, benzoimidazolyl, benzoxazolyl, 1H-benzo[d][1,2,3]triazolyl, and the like.). The heteroaromatic moiety may consist of a single or fused ring system. A typical single heteroaryl ring is a 5- to 6-membered ring containing one to four heteroatoms independently selected from oxygen, sulfur and nitrogen and a typical fused heteroaryl ring system is a 9- to 10-membered ring system containing one to four heteroatoms independently selected from oxygen, sulfur and nitrogen. The fused heteroaryl ring system may consist of two heteroaryl rings fused together or a heteroaryl fused to an aryl (e.g., phenyl).

When the term “heterocycle” is used, it is intended to include “heterocycloalkyl”, “partially or fully saturated heterocycle”, “partially saturated heterocycle”, “fully saturated heterocycle” and “heteroaryl”.

The term “counter ion” is used to represent a negatively charged species such as chloride, bromide, hydroxide, acetate, and sulfate or a positively charged species such as sodium (Na+), potassium (K+), ammonium (RₙNHₘ+, where n=0-4, m=0-4 and m+n=4) and the like.

When a dotted ring is used within a ring structure, this indicates that the ring structure may be saturated, partially saturated or unsaturated.

As referred to herein, the term “substituted” means that at least one hydrogen atom is replaced with a non-hydrogen group, provided that normal valencies are maintained and that the substitution results in a stable compound. When a substituent is keto (i.e., ═O), then 2 hydrogens on the atom are replaced. Keto substituents are not present on aromatic moieties. When a ring system (e.g., carbocyclic or heterocyclic) is said to be substituted with a carbonyl group or a double bond, it is intended that the carbonyl group or double bond be part (i.e., within) of the ring. Ring double bonds, as used herein, are double bonds that are formed between two adjacent ring atoms (e.g., C═C, C═N, or N═N).

In cases wherein there are nitrogen atoms (e.g., amines) on compounds of the present invention, these may be converted to N-oxides by treatment with an oxidizing agent (e.g., mCPBA and/or hydrogen peroxides) to afford other compounds of this invention. Thus, shown and claimed nitrogen atoms are considered to cover both the shown nitrogen and its N-oxide (N→O) derivative.

When any variable occurs more than one time in any constituent or formula for a compound, its definition at each occurrence is independent of its definition at every other occurrence. Thus, for example, if a group is shown to be substituted with 0-3 R groups, then said group may be unsubstituted or substituted with up to three R groups, and at each occurrence R is selected independently from the definition of R.

When a bond to a substituent is shown to cross a bond connecting two atoms in a ring, then such substituent may be bonded to any atom on the ring. When a substituent is listed without indicating the atom in which such substituent is bonded to the rest of the compound of a given formula, then such substituent may be bonded via any atom in such substituent.

Combinations of substituents and/or variables are permissible only if such combinations result in stable compounds.

As a person of ordinary skill in the art would be able to understand, for example, a ketone (—CH—C═O) group in a molecule may tautomerize to its enol form (—C═C—OH). Thus, this invention is intended to cover all possible tautomers even when a structure depicts only one of them.

The phrase “pharmaceutically acceptable” indicates that the substance or composition must be compatible chemically and/or toxicologically, with the other ingredients comprising a formulation, and/or the mammal being treated therewith.

Unless specified otherwise, the term “compounds of the present invention” or “compounds of the present invention” refers to compounds of Formula (IA), as well as isomers, such as stereoisomers (including diastereoisomers, enantiomers and racemates), geometrical isomers, conformational isomers (including rotamers and astropisomers), tautomers, isotopically labeled compounds (including deuterium substitutions), and inherently formed moieties (e.g., polymorphs, solvates and/or hydrates). When a moiety is present that is capable of forming a salt, then salts are included as well, in particular pharmaceutically acceptable salts.

It will be recognized by those skilled in the art that the compounds of the present invention may contain chiral centers and as such may exist in different isomeric forms. As used herein, the term “isomers” refers to different compounds that have the same molecular formula but differ in arrangement and configuration of the atoms.

“Enantiomers” are a pair of stereoisomers that are non-superimposable mirror images of each other. A 1:1 mixture of a pair of enantiomers is a “racemic” mixture. The term is used to designate a racemic mixture where appropriate. When designating the stereochemistry for the compounds of the present invention, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g, (1RS,2RS) as a racemic mixture of (1R,2R) and (1S,2S); (1RS,2SR) as a racemic mixture of (1R,2S) and (1S,2R)). “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Geometric isomers may occur when a compound contains a double bond or some other feature that gives the molecule a certain amount of structural rigidity. If the compound contains a double bond, the substituent may be E or Z configuration. If the compound contains a disubstituted cycloalkyl, the cycloalkyl substituent may have a cis- or trans-configuration.

Conformational isomers (or conformers) are isomers that can differ by rotations about one or more a bonds. Rotamers are conformers that differ by rotation about only a single a bond.

The term “atropisomer” refers to a structural isomer based on axial or planar chirality resulting from restricted rotation in the molecule.

Unless specified otherwise, the compounds of the present invention are meant to include all such possible isomers, including racemic mixtures, optically pure forms and intermediate mixtures. Optically active (R)- and (S)-isomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. using the appropriate solvent or mixture of solvents to achieve good separation).

The present compounds can be isolated in optically active or racemic forms. Optically active forms may be prepared by resolution of racemic forms or by synthesis from optically active starting materials. All processes used to prepare compounds of the present invention and intermediates made therein are considered to be part of the present invention. When enantiomeric or diastereomeric products are prepared, they may be separated by conventional methods, for example, by chromatography or fractional crystallization.

Depending on the process conditions the end products of the present invention are obtained either in free (neutral) or salt form. Both the free form and the salts of these end products are within the scope of the invention. If so desired, one form of a compound may be converted into another form. A free base or acid may be converted into a salt; a salt may be converted into the free compound or another salt; a mixture of isomeric compounds of the present invention may be separated into the individual isomers.

Pharmaceutically acceptable salts are preferred. However, other salts may be useful, e.g., in isolation or purification steps which may be employed during preparation, and thus, are contemplated within the scope of the invention.

As used herein, “pharmaceutically acceptable salts” refer to derivatives of the disclosed compounds wherein the parent compound is modified by making acid or base salts thereof. For example, pharmaceutically acceptable salts include, but are not limited to, acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate/hydroxymalonate, mandelate, mesylate, methylsulphate, mucate, naphthoate, napsylate, nicotinate, nitrate, octadecanoate, oleate, oxalate, palmitate, pamoate, phenylacetate, phosphate/hydrogen phosphate/dihydrogen phosphate, polygalacturonate, propionate, salicylates, stearate, succinate, sulfamate, sulfosalicylate, tartrate, tosylate, trifluoroacetate or xinafoate salt form.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Allen, L. V., Jr., ed., Remington: The Science and Practice of Pharmacy, 22nd Edition, Pharmaceutical Press, London, UK (2012), the disclosure of which is hereby incorporated by reference.

Compounds of the invention that contain groups capable of acting as donors and/or acceptors for hydrogen bonds may be capable of forming co-crystals with suitable co-crystal formers. These co-crystals may be prepared from compounds of the present invention by known co-crystal forming procedures. Such procedures include grinding, heating, co-subliming, co-melting, or contacting in solution compounds of the present invention with the co-crystal former under crystallization conditions and isolating co-crystals thereby formed. Suitable co-crystal formers include those described in WO 2004/078163. Hence the invention further provides co-crystals comprising a compound of the present invention.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The present invention includes various isotopically labeled compounds as defined herein, for example those into which radioactive isotopes, such as ³H, ¹³C, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically labeled compounds of this present invention can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the present invention. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically-labeled compounds of the present disclosure can generally be prepared by conventional techniques known to those skilled in the art or by processes analogous to those described herein, using an appropriate isotopically-labeled reagent in place of the non-labeled reagent otherwise employed. Such compounds have a variety of potential uses, e.g., as standards and reagents in determining the ability of a potential pharmaceutical compound to bind to target proteins or receptors, or for imaging compounds of this disclosure bound to biological receptors in vivo or in vitro.

“Stable compound” and “stable structure” are meant to indicate a compound that is sufficiently robust to survive isolation to a useful degree of purity from a reaction mixture, and formulation into an efficacious therapeutic agent. It is preferred that compounds of the present invention do not contain a N-halo, S(O)₂H, or S(O)H group.

The term “solvate” means a physical association of a compound of this disclosure with one or more solvent molecules, whether organic or inorganic. This physical association includes hydrogen bonding. In certain instances the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Exemplary solvates include, but are not limited to, hydrates, ethanolates, methanolates, and isopropanolates. Methods of solvation are generally known in the art.

As used herein, “polymorph(s)” refer to crystalline form(s) having the same chemical structure/composition but different spatial arrangements of the molecules and/or ions forming the crystals. Compounds of the present invention can be provided as amorphous solids or crystalline solids. Lyophilization can be employed to provide the compounds of the present invention as a solid.

As used herein, the term “patient” encompasses all mammalian species.

As used herein, the term “subject” refers to an animal. Typically the animal is a mammal. A “subject” also refers to any human or non-human organism that could potentially benefit from treatment with a compound of the present invention. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human. Exemplary subjects include human beings of any age with risk factors for infectious diseases.

As used herein, a subject is “in need of” a treatment if such subject would benefit biologically, medically or in quality of life from such treatment (preferably, a human).

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease/disorder refers the treatment of the disease/disorder in a mammal, particularly in a human, and include: (a) ameliorating the disease/disorder, (i.e., slowing or arresting or reducing the development of the disease/disorder, or at least one of the clinical symptoms thereof); (b) relieving or modulating the disease/disorder, (i.e., causing regression of the disease/disorder, either physically, (e.g., stabilization of a discernible symptom), physiologically, (e.g., stabilization of a physical parameter), or both); (c) alleviating or ameliorating at least one physical parameter including those which may not be discernible by the subject; and/or (d) preventing or delaying the onset or development or progression of the disease or disorder from occurring in a mammal, in particular, when such mammal is predisposed to the disease or disorder but has not yet been diagnosed as having it.

As used herein, “preventing” or “prevention” cover the preventive treatment (i.e., prophylaxis and/or risk reduction) of a subclinical disease-state in a mammal, particularly in a human, aimed at reducing the probability of the occurrence of a clinical disease-state. Patients are selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, “risk reduction” or “reducing risk” covers therapies that lower the incidence of development of a clinical disease state. As such, primary and secondary prevention therapies are examples of risk reduction.

“Therapeutically effective amount” is intended to include an amount of a compound of the present invention that will elicit the biological or medical response of a subject, for example, reduction or inhibition of EED and/or PRC2, or ameliorate symptoms, alleviate conditions, slow or delay disease progression, or prevent a disease or disorder mediated by PRC2. When applied to a combination, the term refers to combined amounts of the active ingredients that result in the preventive or therapeutic effect, whether administered in combination, serially, or simultaneously.

Abbreviations as used herein, are defined as follows: “1×” for once, “2×” for twice, “3×” for thrice, “° C.” for degrees Celsius, “aq” for aqueous, “Col” for column, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “nM” for nanomolar, “mol” for mole or moles, “mmol” for millimole or millimoles, “min” for minute or minutes, “h” for hour or hours, “RT” for room temperature, “ON” for overnight, “atm” for atmosphere, “psi” for pounds per square inch, “conc.” for concentrate, “aq” for aqueous, “sat” or “sat'd” for saturated, “MW” for molecular weight, “mw” or “μwave” for microwave, “mp” for melting point, “Wt” for weight, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or ‘tlc’ for thin layer chromatography, “NMR” for nuclear magnetic resonance spectroscopy, “nOe” for nuclear Overhauser effect spectroscopy, “¹H” for proton, “δ” for delta, “s” for singlet, “d” for doublet, “t” for triplet, “q” for quartet, “m” for multiplet, “br” for broad, “Hz” for hertz, “ee” for “enantiomeric excess” and “α”, “β”, “R”, “S”, “E”, and “Z” are stereochemical designations familiar to one skilled in the art.

The following abbreviations used herein below have the corresponding meanings:
Aq aqueous
AlBN azobisisobutyronitrile
Bn benzyl
Boc tert-butoxy carbonyl
Boc₂O di-tert-butyl dicarbonate
Bu butyl
Cs₂CO₃ cesium carbonate anhydrous
CHCl₃ chloroform
DAST diethylaminosulfurtrifluoride
DBU 2,3,4,6,7,8,9,10-octahydropyrimido[1,2-a]azepine
DCM dichloromethane
DIPEA diisopropylethylamine
DMAP 4-dimethylaminopyridine
DME 1,2-dimethoxyethane
DMF dimethylformamide
DMSO dimethylsulfoxide
DPPA diphenylphosphoryl azide
EA ethyl acetate
Equiv. equivalence
Et ethyl
Et₃N triethylamine
Et₂O diethyl ether
EtOH ethanol
EtOAc ethyl acetate
HATU 2-(7-Aza-1H-benzotriazole-1-yl)-1,1,3,3-tetramethyluronium hexafluorophosphate
HCl hydrochloric acid
i-Bu isobutyl
i-Pr isopropyl
KOAc potassium acetate
LiAlH₄ lithium aluminium hydride
Me methyl
MeOH methanol
mCPBA 3-chloroperoxybenzoic acid
MeCN acetonitrile
MnO₂ manganese dioxide
N₂ nitrogen
NaBH₄ sodium borohydride
NaHCO₃ sodium bicarbonate
Na₂SO₄ sodium sulfate
NBS N-Bromosuccinimide
Ph phenyl
PPh₃ triphenylphosphine
Ph₃P═O triphenylphosphine oxide
R_{f} retention factor
RT room temperature (° C.)
t-Bu or Bu^{t} tert-butyl
T3P® Propane phosphonic acid anhydride
TEA triethylamine
TFA trifluoroacetic acid
THF tetrahydrofuran

### IV. Synthesis

The compounds of the present invention can be prepared in a number of ways known to one skilled in the art of organic synthesis in view of the methods, reaction schemes and examples provided herein. The compounds of the present invention can be synthesized using the methods described below, together with synthetic methods known in the art of synthetic organic chemistry, or by variations thereon as appreciated by those skilled in the art. Preferred methods include, but are not limited to, those described below. The reactions are performed in a solvent or solvent mixture appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure.

The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), Larock, R. C., Comprehensive Organic Transformations, 2^{nd}-ed., Wiley-VCH Weinheim, Germany (1999), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the present invention, protection of remote functionality of intermediates may be necessary. The need for such protection will vary depending on the nature of the remote functionality and the conditions of the preparation methods. The need for such protection is readily determined by one skilled in the art. For a general description of protecting groups and their use, see Greene, T. W. et al., Protecting Groups in Organic Synthesis, 4th Ed., Wiley (2007). Protecting groups incorporated in making of the compounds of the present invention, such as the trityl protecting group, may be shown as one regioisomer but may also exist as a mixture of regioisomers.

### General Synthetic Route 1

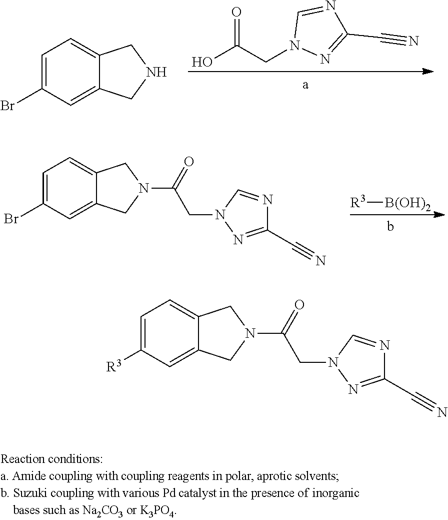

### General Synthetic Route 2

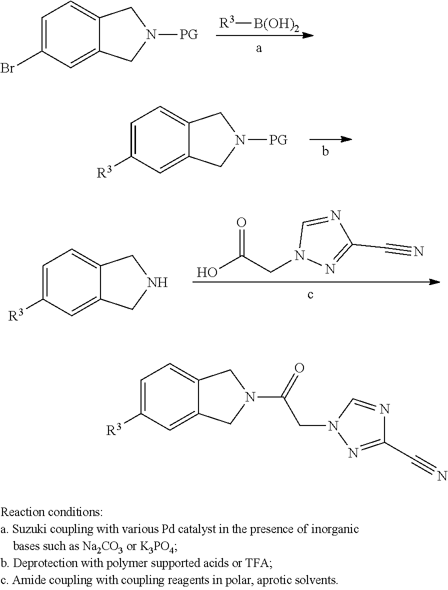

### General Procedures for Amide Coupling

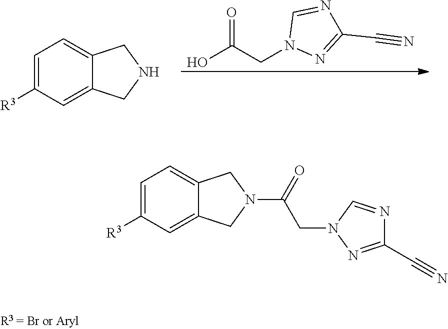

### V. Pharmaceutical Compositions and Combinations

The compounds of the present invention are typically used as a pharmaceutical composition (e.g., a compound of the present invention and at least one pharmaceutically acceptable carrier). A “pharmaceutically acceptable carrier (diluent or excipient)” refers to media generally accepted in the art for the delivery of biologically active agents to animals, in particular, mammals, including, generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, buffering agents (e.g., maleic acid, tartaric acid, lactic acid, citric acid, acetic acid, sodium bicarbonate, sodium phosphate, and the like), disintegration agents, lubricants, sweetening agents, flavoring agents, dyes, and the like and combinations thereof, as would be known to those skilled in the art (see, for example, Allen, L. V., Jr. et al., Remington: The Science and Practice of Pharmacy (2 Volumes), 22nd Edition, Pharmaceutical Press (2012). For purposes of this invention, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above.

The compounds of this disclosure can be administered for any of the uses described herein by any suitable means, for example, orally, such as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

The compound of the present invention is typically formulated into pharmaceutical dosage forms to provide an easily controllable dosage of the drug and to give the patient an elegant and easily handleable product. The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. Compounds of this disclosure may be administered in a single daily dose, or the total daily dosage may be administered in divided doses of two, three, or four times daily.

In certain instances, it may be advantageous to administer the compound of the present invention in combination with at least one additional pharmaceutical (or therapeutic) agent, such as, for treating human African trypanosomiasis, pentamidine, suramin, melarsoprol, eflornithine, fexinidazole and Acoziborole; and for treating Chagas disease, benznidazole, nifurtimox and Amphotericin b; for treating leishmaniasis, meglumine antimoniate, stibogluconate, Amphotericin, Miltefosine, paromomycin or other novel inhibitors in clinical assessment;

The term “combination therapy” refers to the administration of two or more therapeutic agents to treat a therapeutic disease, disorder or condition described in the present invention. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. The compound of the present invention and additional therapeutic agents can be administered via the same administration route or via different administration routes. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The structure of the active compounds identified by code numbers, generic or trade names may be taken from the actual edition of the standard compendium “The Merck Index” or from databases, e.g. Patents International (e.g. IMS World Publications).

In one embodiment, the present invention provides pharmaceutical compositions comprising at least one compound of the present invention (e.g., a compound of the present invention) or a pharmaceutically acceptable salt thereof together with a pharmaceutically acceptable carrier suitable for administration to a human or animal subject, either alone or together with other anti-infective agents.

In one embodiment, In yet another aspect, the present invention is directed to a method for treating, preventing, inhibiting, ameliorating, or eradicating the pathology and/or symptomology of a parasitic disease. The method involves administering to a subject in need thereof, a therapeutically effective amount of a compound or a pharmaceutical composition according to the above embodiments and variations.

In one embodiment of the above method for treating, preventing, inhibiting, ameliorating, or eradicating the pathology and/or symptomology of a parasitic disease, the compound of the invention is capable of inhibiting the proteolytic activity of the proteasome of the parasite causing the parasitic disease.

In another embodiment of the above method for treating, preventing, inhibiting, ameliorating, or eradicating the pathology and/or symptomology of a parasitic disease, the compound of the invention is capable of inhibiting the chymotrypsin-like proteolytic activity of the proteasomes of the parasite causing the parasitic disease.

In another embodiment of the method of the invention, the disease being treated is human African trypanosomiasis, Chagas disease, or leishmaniasis.

In still another embodiment of the method of the invention, the disease being treated is Human African Trypanosomiasis caused by Trypanosoma brucei, particularly, by the sub-species T.b. gambiense or T.b. rhodesiense.

In still another embodiment of the method of the invention, the disease being treated is Chagas disease, (also call American trypanosomiasis) caused by Trypanosoma cruzi.

In still another embodiment of the method of the invention, the disease being treated is Leishmaniasis caused by the parasite Leishmania donovani, Leishmania infantum, Leishmania braziliensis, Leishmania panamensis, Leishmania guayanensis, Leishmania amazonensis, Leishmania mexicana, Leishmania tropica, or Leishmania major.

In still another embodiment of the method of the invention, the disease being treated is visceral Leishmaniasis caused by the parasite Leishmania donovani.

The present invention provides methods of treating a human or animal subject in need of such treatment, comprising administering to the subject a therapeutically effective amount of a compound of the present invention (e.g., a compound of the present invention) or a pharmaceutically acceptable salt thereof, either alone or in combination with other anti-infective agents.

In particular, compositions will either be formulated together as a combination therapeutic or administered separately.

In combination therapy for treatment of infectious diseases, the compound of the present invention and other anti-infective agent(s) may be administered simultaneously, concurrently or sequentially with no specific time limits, wherein such administration provides therapeutically effective levels of the two compounds in the body of the subject.

In a preferred embodiment, the compound of the present invention and the other anti-infective agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present invention and other anti-infective agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another aspect of the present invention, kits that include one or more compound of the present invention and a combination partner as disclosed herein are provided. Representative kits include (a) a compound of the present invention or a pharmaceutically acceptable salt thereof, (b) at least one combination partner, e.g., as indicated above, whereby such kit may comprise a package insert or other labeling including directions for administration.

In another aspect of the present invention, kits that include one or more compound of the present invention and a combination partner as disclosed herein are provided. Representative kits include (a) a compound of the present invention or a pharmaceutically acceptable salt thereof, (b) at least one combination partner, e.g., as indicated above, whereby such kit may comprise a package insert or other labeling including directions for administration.

In the combination therapies of the invention, the compound of the present invention and the other therapeutic agent may be manufactured and/or formulated by the same or different manufacturers. Moreover, the compound of the present invention and the other therapeutic agent (or pharmaceutical agent) may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

### General Methods

The following methods were used in the exemplified Examples, except where noted otherwise.

Amide Coupling Procedure A: T3P® and Et₃N, CH₂Cl₂/DMF

To a suspension of amine (1.0 equiv.) in 5:1 CH₂Cl₂/DMF (0.1 molar) was added 2-(3-cyano-1H-1,2,4-triazol-1-yl)acetic acid (1.0 equiv), T₃P (50% solution in DMF, 1.0 equiv) and Et₃N (3.0 equiv). The resultant mixture was stirred at RT for 18 hours before it was diluted with CH₂Cl₂ and washed with H₂O (twice), 10% LiCl solution (twice) and brine. The organic layers were dried over Na₂SO₄, filtered and concentrated under vacuo to give crude material which was purified by reversed-phase preparative HPLC or silica gel chromatography to provide the desired product.

### Amide Coupling Procedure B: EDC.HCl, HOBT, DIPEA, DMF

To a stirred solution of 2-(3-cyano-1H-1,2,4-triazol-1-yl)acetic acid (1.5 equiv.) in DMF (0.23 molar) was added DIPEA (3.2 equiv.), EDC.HCl (1.5 equiv.) and HOBT (1.5 equiv.). The mixture was stirred at RT for 30 mins before amine (1.0 equiv.) was added. The resultant mixture was stirred at RT for 12 hours before it was diluted with water and extracted with EtOAc (twice). The combined organic layers were washed with water, then brine, dried over Na₂SO₄, filtered and concentrated under vacuo to give crude material which was purified by reversed-phase preparative HPLC or silica gel chromatography to provide the desired product.

### General Procedures for Suzuki Coupling

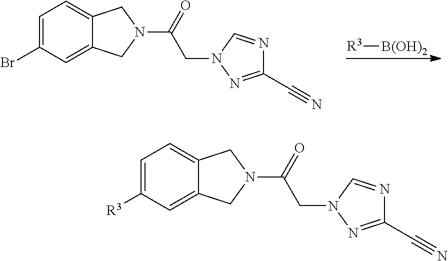

Suzuki Coupling Procedure A: Pd(OAc)₂, Xphos, Na₂CO₃, MeCN/DMF

A microwave vial was charged with aryl bromide (0.20 mmol), arylboronic acid (1.0 equiv.), Pd(OAc)₂ (5 mol %) and Xphos (15 mol %) solvated in degassed 2:1 anhydrous MeCN/DMF (1.5 mL). 1 M aq. Na₂CO₃ solution (2.0 equiv.) was added to the reaction mixture and the resulting mixture was subjected to microwave heating at 120° C. for 15 mins. The reaction mixture was diluted with minimum volume of DMSO (0.5-1 mL) and filtered through Pall's GHO Acrodisc 13 mm syringe filter and subjected directly to reversed-phase preparative HPLC purification.

Suzuki Coupling Procedure B: Pd(Dppf)Cl₂.CH₂Cl₂, Na₂CO₃, DMF

A microwave vial was charged with aryl bromide (0.15 mmol), arylboronic acid (1.0 equiv.), Pd(dppf)Cl₂.CH₂Cl₂ adduct (5 mol %), 1 M aq. Na₂CO₃ solution (2.0 equiv.) and DMF (1.7 mL). The resulting reaction mixture was allowed to purge under Argon for a few minutes before the reaction tube was capped and subjected to microwave heating at 120° C. for 30 mins. The resultant mixture was diluted with DMSO (2 mL), filtered through Pall's GHO Acrodisc 13 mm syringe filter and subjected directly to reversed-phase preparative HPLC purification.

Suzuki Coupling Procedure C: Pd(PPh₃)₂Cl₂, Na₂CO₃, 1,4-dioxane

Aryl bromide (0.064 mmol), arylboronic acid (1.3 equiv.) and Pd(PPh₃)₂Cl₂ (5 mol %) were weighed into a microwave vial and the microwave vial was evacuated and backfilled with argon. Anhydrous 1,4-dioxane (0.1 molar) was added and 1 M aq. Na₂CO₃ solution (2.0 equiv.) was added. The reaction mixture was subjected to microwave heating at 120° C. for 20 mins. The resultant mixture was passed through a 2 g Si-carbonate cartridge (pre-equilibrated with methanol) to scavenge off excess boronic acid starting material. The cartridge was washed with MeOH (10 mL) and the eluent was collected and concentrated at reduced pressure. The crude material was solvated in MeOH (1 mL), filtered through Pall's GHO Acrodisc 13 mm syringe filter and subjected directly to reversed-phase preparative HPLC purification.

Suzuki Coupling Procedure D: Pd(OAc)₂, Xphos, Na₂CO₃, THF/DMF

Aryl bromide (0.090 mmol) solvated in anhydrous THF (0.5 mL) and DMF (0.25 mL) was added to a microwave vial containing arylboronic acid (1.3 equiv.). Pd(OAc)₂ (10 mol %) and Xphos (30 mol %) solvated in anhydrous THF (0.5 mL) and DMF (0.25 mL) was added to the reaction mixture followed by 1 M aq. Na₂CO₃ solution (2.5 equiv.). The reaction mixture was subjected to microwave heating at 120° C. for 10 mins before the resultant mixture was filtered through Pall's GHO Acrodisc 13 mm syringe filter and subjected directly to reversed-phase preparative HPLC purification.

Suzuki Coupling Procedure E: Pd(OAc)₂, Xphos, Na₂CO₃, MeCN/DMF

Aryl bromide (0.090 mmol) solvated in anhydrous MeCN (0.8 mL) and DMF (0.4 mL) was added to a microwave vial containing arylboronic acid (1.3 equiv.). 1 M aq. Na₂CO₃ solution (2.0 equiv.) was added followed by Pd(OAc)₂ (10 mol %) and Xphos (30 mol %) solvated in anhydrous THF (0.1 mL) and DMF (0.1 mL). The reaction mixture was subjected to microwave heating at 100° C. for 10 mins before the resultant mixture was filtered through Pall's GHO Acrodisc 13 mm syringe filter and subjected directly to reversed-phase preparative HPLC purification.

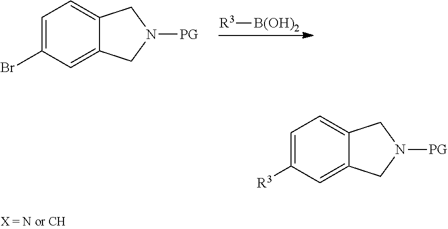

Suzuki Coupling Procedure F: Pd(Dppf)Cl₂.CH₂Cl₂, K₃PO₄, 1,4-dioxane/H₂O

To a sealed tube containing aryl bromide (1.0 equiv.) in 7:3 1,4-dioxane/H₂O (0.23 molar) was added K₃PO₄ (1.5 equiv.), arylboronic acid (1.5 equiv.) and Pd(dppf)Cl₂.CH₂Cl₂ (5 mol %). The reaction mixture was heated at 100° C. for 6 hours before it was diluted with H₂O and extracted with EtOAc (twice). The combined organic layers were washed with H₂O, then brine, dried over Na₂SO₄, filtered and concentrated under vacuo to give crude material which was purified by silica gel chromatography to provide the desired product.

Suzuki Coupling Procedure G: Pd(Dppf)Cl₂.CH₂Cl₂, Na₂CO₃, 1,4-dioxane/H₂O

To a sealed tube containing aryl bromide (1.0 equiv.) in 7:3 1,4-dioxane/H₂O was added Na₂CO₃ (1.5-3.0 equiv.), arylboronic acid/ester (1.5 equiv.) and Pd(dppf)Cl₂.CH₂Cl₂ (5 mol %). The reaction mixture was heated at 100° C. for 6 hours before it was diluted with H₂O and extracted with EtOAc (twice). The combined organic layers were washed with H₂O, then brine, dried over Na₂SO₄, filtered and concentrated under vacuo to give crude material which was purified by silica gel chromatography to provide the desired product.

Suzuki Coupling Procedure H: Pd(PPh₃)₂Cl₂, K₂CO₃, DMF/H₂O

To a sealed tube containing aryl bromide (1.0 equiv.) in 1:1 DMF/H₂O was added K₂CO₃ (3.0 equiv.), arylboronic acid/ester (1.2 equiv.) and Pd(PPh₃)₂Cl₂ (5 mol %). The reaction mixture was heated at 60° C. for 5 hours before it was diluted with H₂O and extracted with EtOAc (twice). The combined organic layers were washed with H₂O, then brine, dried over Na₂SO₄, filtered and concentrated under vacuo to give crude material which was purified by silica gel chromatography to provide the desired product.

Suzuki Coupling Procedure I: Pd(dtbpf)Cl₂, K₃PO₄, Dioxane/H₂O

Aryl bromide (1.0 equiv.) solvated in 4:1 Dioxane/H₂O was added to a microwave vial containing arylboronic acid/ester (1.3 equiv.). K₃PO₄ (2.5 equiv.) was added to the reaction mixture followed by Pd(dtbpf)Cl₂ (10 mol %). The reaction mixture was subjected to microwave heating at 120° C. for 30 mins before the resultant mixture was filtered through celite bed and filtrate was evaporated in vacuo to give crude material which was purified by silica gel chromatography to provide the desired product.

Suzuki Coupling Procedure J: Pd(Dppf)Cl₂.CH₂Cl₂, TEA, MeOH

To a sealed tube containing aryl bromide (1.0 equiv.) in MeOH (40 vol) was added TEA (2.5 equiv.) arylboronic acid/ester (1.0 equiv.) and Pd(dppf)Cl₂.CH₂Cl₂ (10 mol %). The reaction mixture was heated at 100° C. for 10 hours before it was diluted with H₂O and extracted with EtOAc (twice). The combined organic layers were washed with H₂O, then brine, dried over Na₂SO₄, filtered and concentrated under vacuo to give crude material which was purified by silica gel chromatography to provide the desired product.

Suzuki Coupling Procedure K: Pd(PPh₃)₄, K₂CO₃, 1,4-dioxane/H₂O

To a sealed tube containing aryl bromide (1.0 equiv.) in 4:1 1,4-dioxane/H₂O was added K₂CO₃/K₃PO₄ (1.5 equiv.), arylboronic acid/ester (1.5 equiv.) and Pd(PPh₃)₄ (5 mol %). The reaction mixture was heated at 100° C. for 6 hours before it was diluted with H₂O and extracted with EtOAc (twice). The combined organic layers were washed with H₂O, then brine, dried over Na₂SO₄, filtered and concentrated under vacuo to give crude material which was purified by silica gel chromatography to provide the desired product.

### General Procedures for Deprotection

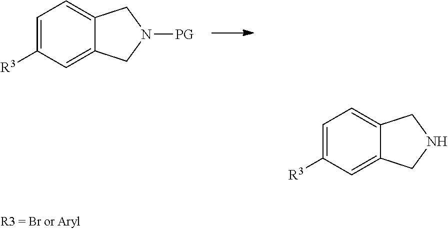

Deprotection Procedure A: SCX-2 Resin, CH₂Cl₂

SCX-2 resin (0.6 mmol/g, 1.5 equiv.) was added to the Boc-protected amine (1.0 equiv.) solvated in CH₂Cl₂ (0.20 molar). The mixture was heated at 60° C. for 1 hour before it was filtered through an empty cartridge. The SCX-2 resin was collected and washed with CH₂Cl₂ (twice) to remove impurities present before the desired product was released from the resin using 2 M NH₃ in EtOH (5 mL). The eluent was collected and concentrated under reduced pressure to obtain the free amine.

Deprotection Procedure B: TFA, CH₂Cl₂

Trifluoroacetic acid (20 equiv.) was added to the Boc-protected amine (1.0 equiv.) solvated in CH₂Cl₂ (0.17 molar). The reaction mixture was stirred at RT for 2 hours before it was added to a saturated NaHCO₃ solution. The organic product was extracted with CH₂Cl₂ (thrice) and the organic layers were combined, washed with H₂O and separated through a phase separator cartridge. The organic layer was concentrated under reduced pressure to obtain the free amine.

Deprotection Procedure C: TFA, CH₂Cl₂/MeOH

Trityl-protected amine (1.0 equiv.) was dissolved in 1:1 CH₂Cl₂/MeOH (0.20 molar) and trifluoroacetic acid (30 equiv.) was added at 0° C. The resultant mixture was warmed to RT and stirred at RT for 2 hours before it was concentrated under vacuo. The crude material was washed with 10% Et₂O in n-Pentane to give the product as a TFA salt.

Purification of intermediates and final products was carried out via either normal or reverse phase chromatography. Normal phase chromatography was carried out using prepacked SiO₂ cartridges eluting with either gradients of hexanes and ethyl acetate or DCM and MeOH unless otherwise indicated. For highly polar amines, gradients of DCM and 1 M NH₃ in MeOH were used. Reverse phase preparative HPLC was carried out using C18 columns with UV 214 nm and 254 nm or prep LCMS detection eluting with gradients of Solvent A (water with 0.1% TFA) and Solvent B (acetonitrile with 0.1% TFA) or with gradients of Solvent A (water with 0.05% TFA) and Solvent B (acetonitrile with 0.05% TFA) or with gradients of Solvent A (water with 0.05% ammonia) and Solvent B (acetonitrile with 0.05% ammonia).

### LC/MS Methods Employed in Characterization of Examples

Reversed-phase analytical HPLC/MS was performed on Waters Acquity UPLC system coupled with ZQ detector (Method A), or Shimadzu LCMS-8030 system (Method B).
Method A: Linear gradient of 5% to 98% B over 1.4 min, with 0.4 min hold at 98% B followed by 0.2 min linear gradient from 98% to 5% B;

UV visualization at 214 nm and 254 nm

Column: Acquity HSS T3 1.8 μm 2.1×50 mm at 60° C.

Flow rate: 1.0 mL/min

Solvent A: 0.05% formic acid, 99.95% water

Solvent B: 0.04% formic acid, 99.96% acetonitrile.
Method B: Linear gradient of 5% to 95% B over 0.5 min, with 0.5 min hold at 95% B followed by 0.5 min linear gradient from 95% to 5% B;

UV visualization at 214 nm and 254 nm

Column: Mercury MS Synergi 2.5μ C18, 20×4.0 mm at 40° C.

Flow rate: 2.0 mL/min

Solvent A: 0.1% formic acid, 99.9% water

Solvent B: acetonitrile

### Preparative HPLC Methods Employed in Purification of Examples

Reversed-phase preparative HPLC was performed on Agilent 1200 Series (Method A, B, C, D and E) and WATERS Mass Directed Auto Purification System (Method F-I).
Method A: Linear Gradient of 50% to 60% B over 2.0 min, followed by linear gradient of 60% to 80% B over 3.0 min;

UV visualization at 210 nm

Column: KINETEX EVO 5μ C18 (21.2 mm×150 mm)

Flow rate: 18.0 mL/min

Solvent A: Water

Solvent B: Acetonitrile
Method B: Linear Gradient of 30% to 35% B over 2.0 min, followed by linear gradient of 35% to 60% B over 8.0 min;

UV visualization at 210 nm

Column: ZORBAX 5μ C18 (21.2 mm×150 mm)

Flow rate: 20.0 mL/min

Solvent A: Water

Solvent B: Acetonitrile
Method C: Linear Gradient of 30% to 35% B over 2.0 min, followed by linear gradient of 35% to 60% B over 8.0 min;

UV visualization at 210 nm

Column: X BRIDGE 5μ C18 (21.2 mm×150 mm)

Flow rate: 20.0 mL/min

Solvent A: Water

Solvent B: Acetonitrile
Method D: Linear Gradient of 20% to 30% B over 2.0 min, followed by linear gradient of 30% to 70% B over 7.0 min;

UV visualization at 210 nm

Column: WATERS XBRIDGE C18, (21.2 mm×150 mm)

Flow rate: 20.0 mL/min

Solvent A: Water

Solvent B: Acetonitrile
Method E: Linear Gradient of 20% to 30% B over 2.0 min, followed by linear gradient of 30% to 80% B over 6.0 min;

UV visualization at 210 nm

Column: kinetex 5μ C18 (21.2 mm×150 mm)

Flow rate: 20.0 mL/min

Solvent A: Water

Solvent B: Acetonitrile
Method F: 1.0 min hold at 40% B, followed by linear gradient of 40% to 70% B over 6.0 min, followed by linear gradient of 70% to 100% B over 0.1 min, with 0.8 min hold at 100% B followed by 0.1 min linear gradient from 100% to 5% B;

UV visualization at 254 nm

Column: WATERS Xselect CSH Prep C18 OBD, 5 μm, 30×100 mm

Flow rate: 50.0 mL/min

Solvent A: 5% formic acid, 95% water

Solvent B: acetonitrile
Method G: 1.0 min hold at 40% B, followed by linear gradient of 40% to 70% B over 6.0 min, followed by linear gradient of 70% to 100% B over 0.1 min, with 0.8 min hold at 100% B followed by 0.1 min linear gradient from 100% to 5% B; UV visualization at 254 nm

Column: WATERS Sunfire Prep C18 OBD, 5 μm, 19×100 mm

Flow rate: 20.0 mL/min

Solvent A: 0.1% formic acid, 99.9% water

Solvent B: acetonitrile
Method H: 1.0 min hold at 30% B, followed by linear gradient of 30% to 55% B over 6.0 min, followed by linear gradient of 55% to 100% B over 0.1 min, with 0.8 min hold at 100% B followed by 0.1 min linear gradient from 100% to 5% B;

UV visualization at 254 nm

Column: WATERS XBridge Prep C18 OBD, 5 μm, 19×100 mm

Flow rate: 20.0 mL/min

Solvent A: 0.1% formic acid, 99.9% water

Solvent B: acetonitrile
Method I: 1.0 min hold at 60% B, followed by linear gradient of 60% to 85% B over 6.0 min, followed by linear gradient of 85% to 100% B over 0.1 min, with 0.8 min hold at 100% B followed by 0.1 min linear gradient from 100% to 5% B; UV visualization at 254 nm

Column: WATERS Sunfire Prep C18 OBD, 5 μm, 19×100 mm

Flow rate: 20.0 mL/min

Solvent A: 0.1% formic acid, 99.9% water

Solvent B: acetonitrile

### NMR Employed in Characterization of Examples

¹H NMR spectra were obtained with Bruker Fourier transform spectrometers operating at frequencies as follows: ¹H NMR: 400 MHz (Bruker). ¹³C NMR: 100 MHz (Bruker). Spectra data are reported in the format: chemical shift (multiplicity, number of hydrogens). Chemical shifts are specified in ppm downfield of a tetramethylsilane internal standard (δ units, tetramethylsilane=0 ppm) and/or referenced to solvent peaks, which in ¹H NMR spectra appear at 2.49 ppm for CD₂HSOCD₃, 3.30 ppm for CD₂HOD, 1.94 for CD₃CN, and 7.24 ppm for CDCl₃, and which in ¹³C NMR spectra appear at 39.7 ppm for CD₃SOCD₃, 49.0 ppm for CD₃OD, and 77.0 ppm for CDCl₃. All ¹³C NMR spectra were proton decoupled.

### V. Examples

The following Examples have been prepared, isolated and characterized using the methods disclosed herein. The following examples demonstrate a partial scope of the disclosure and are not meant to be limiting of the scope of the disclosure.

Unless specified otherwise, starting materials are generally available from a non-excluding commercial sources such as TCl Fine Chemicals (Japan), Shanghai Chemhere Co., Ltd. (Shanghai, China), Aurora Fine Chemicals LLC (San Diego, Calif.), FCH Group (Ukraine), Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), AstraZeneca Pharmaceuticals (London, England), Chembridge Corporation (USA), Matrix Scientific (USA), Conier Chem & Pharm Co., Ltd (China), Enamine Ltd (Ukraine), Combi-Blocks, Inc. (San Diego, USA), Oakwood Products, Inc. (USA), Apollo Scientific Ltd. (UK), Allichem LLC. (USA) and Ukrorgsyntez Ltd (Latvia).

### Synthesis of Intermediates

### Intermediate I-1: Synthesis of Allyl 2-bromoacetate

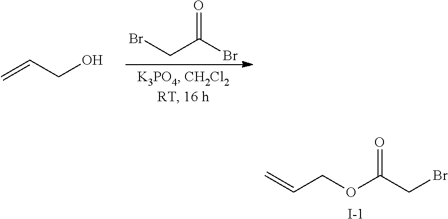

To a stirred solution of allyl alcohol (26.8 g, 461 mmol) in CH₂Cl₂ (200 mL) was added K₃PO₄ (245 g, 1154 mmol, 2.5 equiv.) and 2-bromoacetyl bromide (139 g, 689 mmol, 1.5 equiv.) at 0° C. The resultant mixture was allowed to warm to RT and stirred at RT for 16 hours before it was filtered through a bed of celite. The filtrate was concentrated under vacuo to give I-1 (75.0 g, 91% yield) as colorless oil. The crude material was used in the next step without further purification. R_{f}=0.80 (30% EtOAc/hexanes); ¹H NMR (300 MHz, CDCl₃) δ 5.98-5.85 (m, 1H), 5.40-5.26 (m, 2H), 4.68-4.65 (m, 2H), 3.86 (s, 2H).

### Intermediate I-2: Synthesis of Allyl 2-(3-cyano-1H-1,2,4-triazol-1-yl)acetate

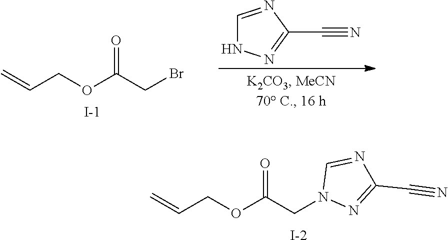

To a stirred solution of 1H-1,2,4-triazole-3-carbonitrile (25.0 g, 266 mmol) in MeCN (300 mL) was added K₂CO₃ (73.5 g, 532 mmol, 2.0 equiv.) and I-1 (52.4 g, 293 mmol, 1.1 equiv.) at 0° C. The resultant mixture was heated at 70° C. for 16 hours before it was filtered through a bed of celite. The filtrate was concentrated under vacuo to give a crude material which was purified by silica gel chromatography to afford I-2 (25.0 g, 49% yield) as a white solid. R_{f}=0.50 (30% EtOAc/hexanes); ¹H NMR (300 MHz, CDCl₃) δ 8.32 (s, 1H), 5.97-5.83 (m, 1H), 5.39-5.30 (m, 2H), 5.07 (s, 2H), 4.73-4.69 (m, 2H).

### Intermediate I-3: Synthesis of 2-(3-cyano-1H-1,2,4-triazol-1-yl)acetic Acid

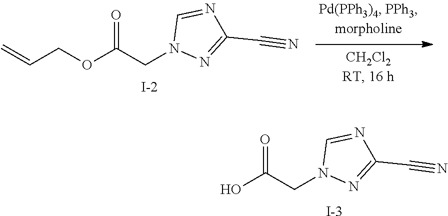

To a solution of I-2 (25.0 g, 130 mmol) in CH₂Cl₂ (550 mL) was added morpholine (11.4 mL, 130 mmol, 1.0 equiv.), PPh₃ (17.0 g, 65.0 mmol, 0.5 equiv.) and Pd(PPh₃)₄ (15.0 g, 13 mmol, 0.1 equiv.). The resultant mixture was stirred at RT for 16 hours before it was concentrated under vacuo. The residue was taken up in acetone (200 mL) and filtered through a bed of celite to remove triphenylphosphonium salt. The filtrate was concentrated under vacuo to give a partially purified product which was then dissolved in H₂O (500 mL) and extracted with EtOAc (2×500 mL). The aqueous layer was acidified to pH ˜6 with solid citric acid and extracted with 10% MeOH in CH₂Cl₂ (2×500 mL). The combined organic layers was washed with brine, dried over Na₂SO₄, filtered and concentrated under vacuo to afford I-3 (15.0 g, 76% yield) with traces of TPP salt as a brown residue. R_{f}=0.23 (10% MeOH/CH₂Cl₂); ¹H NMR (300 MHz, CDCl₃) δ 8.32 (s, 1H), 5.0 (s, 2H); LC-MS m/z 151 [M−H]⁻ (Method B).

### Intermediate I-4: Synthesis of tert-butyl 5-(4,4,5,5-tetramethyl-1,3,2-dioxaborolan-2-yl)isoindoline-2-carboxylate (I-4)

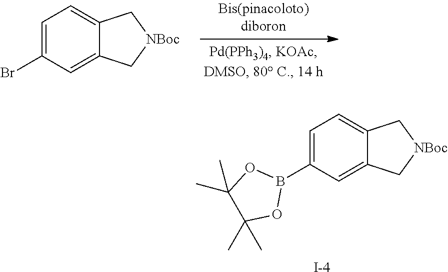

To a seal tube containing tert-butyl 5-bromoisoindoline-2-carboxylate (20 g, 67.09 mmol, 1.0 equiv.) in DMSO (200 mL) was added KOAc (26.34 g, 286.36 mmol, 4.0 equiv.), bispinacolatodiboron (34.07 g, 134.18 mmol, 2.0 equiv.) and degassed with argon for 10 min. Pd(PPh₃)₄ (7.75 g, 6.70 mmol, 10 mol %) was added, sealed the tube and the reaction mixture was heated at 80° C. for 10 hours before it was diluted with H₂O and extracted with EtOAc (twice). The combined organic layers were washed with H₂O, then brine, dried over Na₂SO₄, filtered and concentrated under vacuo to give crude material which was purified by silica gel chromatography using 5% EtOAc in hexanes to provide the desired product I-4 22.0 g (95.03%) as white solid. Rf=0.6 (10% EtOAC in Hexane). ¹H NMR (300 MHz, Chloroform-d) δ 7.75-7.65 (m, 2H), 7.30-7.20 (m, 1H), 4.77-4.57 (m, 4H), 1.51 (s, 9H), 1.34 (s, 12H); HPLC: 95.62%, retention time=6.38 min (Method D).

### Intermediate I-5: Synthesis of 1-(2-(5-bromoisoindolin-2-yl)-2-oxoethyl)-1H-1,2,4-triazole-3-carbonitrile

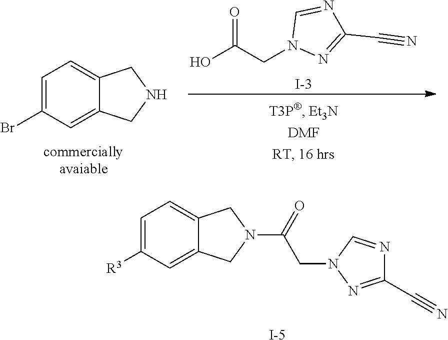

To a solution of amine (2.46 g, 12.4 mmol) in DMF (25 mL) was added acid I-3 (1.89 g, 12.4 mmol, 1.0 equiv), T3P® (9.61 mL, 50% solution in DMF, 1.3 equiv.) and Et₃N (3.46 mL, 24.8 mmol, 2.0 equiv.). The resultant mixture was stirred at RT for 16 hours before 10% LiCl solution was added. The mixture was extracted with EtOAc (5×100 mL) and the combined organic layers were washed with brine, dried over MgSO₄, filtered and concentrated under vacuo. The crude material was purified by silica gel chromatography (20% MeOH/CH₂Cl₂) to afford I-5 (2.37 g, 55% yield) as a grey solid. ¹H NMR (400 MHz, DMSO-d6) δ 8.87 (d, J=1.6 Hz, 1H), 7.64 (d, J=5.8 Hz, 1H), 7.52 (d, J=8.1 Hz, 1H), 7.37 (dd, J=8.2, 4.2 Hz, 1H), 5.49 (s, 2H), 4.94 (d, J=15.9 Hz, 2H), 4.68 (d, J=17.9 Hz, 2H); LC-MS m/z 332, 334 [M+H]⁺, retention time=0.90 min (Method A).

### Example 1: 1-(2-(5-(2-(difluoromethyl)pyridin-3-yl)isoindolin-2-yl)-2-oxoethyl)-1H-1,2,4-triazole-3-carbonitrile

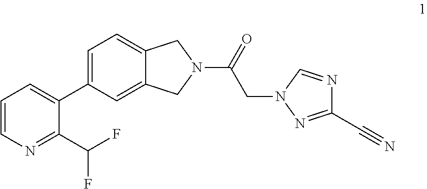 [SMILES: N#Cc1ncn(CC(=O)N2Cc3ccc(-c4cccnc4C(F)F)cc3C2)n1]

### Preparation of 3-bromo-2-(difluoromethyl)pyridine

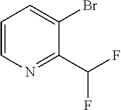

To a solution of 3-bromopicolinaldehyde (0.50 g, 2.68 mmol) in dichloromethane (10 mL) was added Diethyl aminosulfur trifluoride (DAS, 0.86 g, 5.37 mmol) drop wise at 0° C. The reaction mixture was stirred for 3 hours at 0° C. Then quenched with saturated sodium bicarbonate solution very carefully at 0° C. and extracted with dichloromethane (10 mL×3). The combined organic layer was dried over sodium sulphate and concentrated in vacuo. The crude compound was purified by Column chromatography using 0-30% EtOAc in n-Hexane to afford title compound as colourless liquid (0.25 g, 44.84%) Rf=0.9 (30% EtOAc in n-Hexane); Rf=0.90 (30% EtOAc in n-Hexane); ¹H NMR (300 MHz, Chloroform-d) δ 8.65 (dd, J=4.7, 1.4 Hz, 1H), 7.98 (dd, J=8.1, 1.2 Hz, 1H), 7.32 (dd, J=8.1, 4.6 Hz, 1H), 6.92 (t, J=53.9 Hz, 1H).

### Preparation of tert-butyl 5-(2-(difluoromethyl)pyridin-3-yl)isoindoline-2-carboxylate

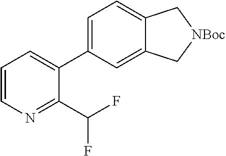 [SMILES: CC(C)(C)OC(=O)N1Cc2ccc(-c3cccnc3C(F)F)cc2C1]

Building block was prepared from intermediate I-4 using Suzuki coupling procedure F and 3-bromo-2-(difluoromethyl)pyridine. The crude compound was purified by column chromatography using 0-30% EtOAc in n-hexanes to afford title compound as colourless liquid 0.1 g (59.80%, yield) ¹H NMR (300 MHz, Chloroform-d) δ 8.83-8.69 (m, 1H), 7.71 (d, J=7.8 Hz, 1H), 7.58-7.30 (m, 2H), 7.26 (s, 2H), 6.78-6.34 (m, 1H), 4.76 (s, 2H), 4.73 (s, 2H), 1.60 (s, 9H); HPLC: 85.68%, retention time=7.55 min (Method E).

### Preparation of 5-(2-(difluoromethyl)pyridin-3-yl)isoindoline trifluoroacetic Acid Salt

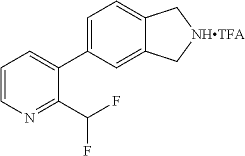

Amine was prepared from previous building block (0.1 g) using Boc-deprotection procedure A. The crude material taken as such for next step (0.1 g). LC-MS m/z 247.00 [M+H]⁺, retention time=0.87 min (Method A).

### Preparation of 1-(2-(5-(2-(difluoromethyl)pyridin-3-yl)isoindolin-2-yl)-2-oxoethyl)-1H-1,2,4-triazole-3-carbonitrile (1)

Compound 1 was prepared from intermediate I-3 using acid amide coupling procedure A. The crude compound was purified by column chromatography using 0-80% EtOAc in n-hexanes, further purified by recrystallisation in ethyl acetate to afford title compound as tan solid 0.04 g (36.26%, yield). ¹H NMR (600 MHz, Chloroform-d) δ 8.75 (s, 1H), 8.47 (d, J=3.0 Hz, 1H), 7.70 (s, 1H), 7.53-7.33 (m, 4H), 6.60 (t, J=54.0 Hz, 1H), 5.17 (s, 2H), 5.04 (s, 2H), 4.93 (s, 2H); LC-MS m/z 381.00 [M+H]⁺, retention time=1.42 min (Method A); HPLC: 97.37%, retention time=6.16 min (Method E).

### Example 2: 1-(2-(5-(3-chloro-5-(trifluoromethyl)pyridin-4-yl)isoindolin-2-yl)-2-oxoethyl)-1H-1,2,4-triazole-3-carbonitrile

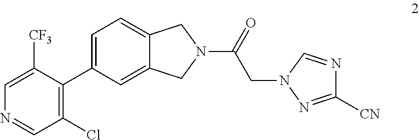 [SMILES: N#Cc1ncn(CC(=O)N2Cc3ccc(-c4c(Cl)cncc4C(F)(F)F)cc3C2)n1]

### Preparation of 3-chloro-5-(trifluoromethyl)pyridin-2-amine

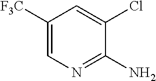 [SMILES: Nc1ncc(C(F)(F)F)cc1Cl]

A solution of 2,3-dichloro-5-(trifluoromethyl)pyridine (1.0 g, 4.65 mmol) in Aq. NH₃ (0.5 mL) was stirred at 100° C. for 12 h in a sealed tube. Reaction mixture was concentrated to dryness and was purified by combi flash column chromatography using 70% EtOAc in n-hexanes to afford title compound 0.8 g (90.0%), Rf=0.1 (70% EtOAc in n-Hexane); ¹H NMR (300 MHz, Chloroform-d) δ 8.23 (d, J=1.8 Hz, 1H), 7.70 (d, J=1.8 Hz, 1H); LC-MS m/z 196.9 [M+H]⁺, retention time=0.98 min (Method D); HPLC: 95.32%, retention time=6.53 min (Method E).

### Preparation of N-(3-chloro-5-(trifluoromethyl)pyridin-2-yl)pivalamide

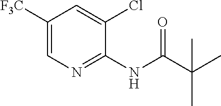 [SMILES: CC(C)(C)C(=O)Nc1ncc(C(F)(F)F)cc1Cl]

To a stirred solution of precursor (400 mg, 2.04 mmol) in DCM (10 mL) was added pyridine (480 mg, 6.12 mmol), followed by dropwise addition of pivaloyl chloride (730 mg, 6.12 mmol) at 0° C. and stirred at 40° C. for 48 h. Reaction mixture was quenched with water and extracted with EtOAc (10 mL×2). Combined organic layer was washed with brine, dried over anhydrous Na₂SO₄ and concentrated to obtain crude product. Product was purified by combi flash column chromatography using 35% EtOAc in n-hexanes to afford title compound 0.1 g (17.45%), Rf=0.5 (30% EtOAc in n-Hexane); LC-MS m/z 281.10 [M+H]⁺, retention time=1.49 min (Method A); HPLC: 97.85%, retention time=3.94 min (Method D).

### Preparation of N-(3-chloro-4-iodo-5-(trifluoromethyl)pyridin-2-yl)pivalamide

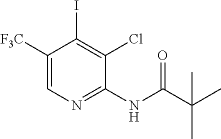 [SMILES: CC(C)(C)C(=O)Nc1ncc(C(F)(F)F)c(I)c1Cl]

To a stirred solution of precursor (800 mg, 2.85 mmol) in dry THF (5 mL) was added 2M LDA in Hexanes (5.7 mL, 11.40 mmol) at −78° C., maintained for 2 h at the same temperature. To the above reaction mass, Iodine (2.89 g, 11.40 mmol) in dry THF (5 mL) was added dropwise and stirred at −78° C. for 30 min. Reaction mixture was quenched with ice cold water at −78° C. and extracted with EtOAc (20 mL×2). Combined organic layer was washed with brine, dried over anhydrous Na₂SO₄ and concentrated to obtain crude product. Product was purified by combi flash column chromatography using 3% EtOAc in n-hexanes to afford title compound 0.840 g (72.48%), Rf=0.6 (05% EtOAc in n-Hexane); ¹H NMR (300 MHz, Chloroform-d) δ 8.51 (s, 1H), 8.34 (s, 1H), 1.36 (s, 9H); LC-MS m/z 406.8 [M+H]⁺, retention time=2.48 min (Method G).

### Preparation of 3-chloro-4-iodo-5-(trifluoromethyl)pyridin-2-amine

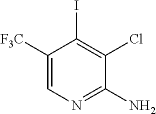 [SMILES: Nc1ncc(C(F)(F)F)c(I)c1Cl]

A stirred solution of precursor (300 mg, 0.73 mmol) in 20% Aqueous H₂SO₄ (10 mL) was stirred at 100° C. for 16 h. Reaction mixture was quenched with aq. ammonia and extracted with EtOAc (10 mL×2). Combined organic layer was washed with brine, dried over anhydrous Na₂SO₄ and concentrated to obtained crude product of 0.25 g, Rf=0.3 (10% EtOAc in n-Hexane); ¹H NMR (300 MHz, Chloroform-d) δ 8.10 (s, 1H), 5.40 (s, 2H); LC-MS m/z 322.9 [M+H]⁺, retention time=1.48 min (Method D); HPLC: 94.41%, retention time=6.93 min (Method E).

### Preparation of 3-chloro-4-iodo-5-(trifluoromethyl)pyridine

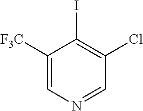 [SMILES: FC(F)(F)c1cncc(Cl)c1I]

To a stirred solution of precursor (430 mg, 1.33 mmol) in THF (4 mL) was added t-butyl nitrite (0.4 mL) and stirred at 65° C. for 10 min. Reaction mixture was concentrated to obtain crude product. Product was purified by combi flash column chromatography using 3% EtOAc in n-hexanes to afford title compound 0.20 g (48.78%), Rf=0.4 (10% EtOAc in n-Hexane); ¹H NMR (400 MHz, Chloroform-d) δ 8.70 (s, 1H), 8.57 (s, 1H); LC-MS m/z 307.9 [M+H]⁺, retention time=2.20 min (Method G); HPLC: 80.73%, retention time=4.43 min (Method D).

### Preparation of tert-butyl 5-(3-chloro-5-(trifluoromethyl)pyridin-4-yl)isoindoline-2-carboxylate

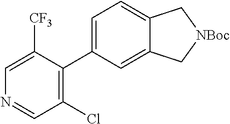 [SMILES: CC(C)(C)OC(=O)N1Cc2ccc(-c3c(Cl)cncc3C(F)(F)F)cc2C1]

Title compound was prepared from intermediate I-4 using Suzuki coupling procedure F and precursor (200 mg, 0.65 mmol). The crude compound was purified by column chromatography using 0-15% EtOAc in n-hexanes to afford title compound (100 mg, 38.61%). ¹H NMR (300 MHz, Chloroform-d) δ 8.87 (s, 2H), 7.40-7.32 (m, 1H), 7.18-6.94 (m, 2H), 4.87-4.60 (m, 4H), 1.52 (d, J=1.5 Hz, 9H); LC-MS m/z 398.9 [M+H]⁺, retention time=2.44 min (Method I); HPLC: 91.34%, retention time=7.74 min (Method F).

### Preparation of 5-(3-chloro-5-(trifluoromethyl)pyridin-4-yl)isoindoline

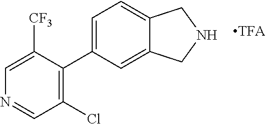 [SMILES: FC(F)(F)c1cncc(Cl)c1-c1ccc2c(c1)CNC2]

Title compound was prepared from precursor (100 mg, 0.25 mmol) using Boc-deprotection procedure A. The crude material was taken to next step as such (150 mg). HPLC: 93.06%, retention time=5.23 min (Method E).

### Preparation of 1-(2-(5-(3-chloro-5-(trifluoromethyl)pyridin-4-yl)isoindolin-2-yl)-2-oxoethyl)-1H-1,2,4-triazole-3-carbonitrile (2)

Example 2 was prepared from intermediate I-3 using acid amide coupling procedure A and precursor (150 mg, 0.502 mmol). The crude compound was purified by column chromatography using 1% MeOH in DCM to afford title compound as off white solid (50 mg, 23.04%). ¹H NMR (300 MHz, Chloroform-d) δ 8.89 (s, 2H), 8.46 (s, 1H), 7.45 (t, J=7.4 Hz, 1H), 7.25-7.16 (m, 2H), 5.17 and 5.16 (s, 2H), 5.05 and 5.04 (s, 2H), 4.95 and 4.93 (s, 2H) (NMR showing doubling of protons due to presence of rotamers); LC-MS m/z 433.0 [M+H]⁺, retention time=2.03 min (Method I); HPLC: 95.76%, retention time=7.25 min (Method B).

### Example 3: 1-(2-(5-(5-chloro-2-fluoropyridin-4-yl)isoindolin-2-yl)-2-oxoethyl)-1H-1,2,4-triazole-3-carbonitrile

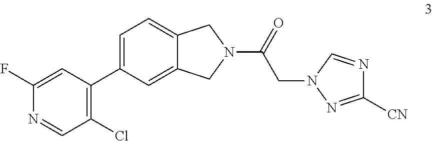 [SMILES: N#Cc1ncn(CC(=O)N2Cc3ccc(-c4cc(F)ncc4Cl)cc3C2)n1]

### Preparation of tert-butyl 5-(5-chloro-2-fluoropyridin-4-yl)isoindoline-2-carboxylate

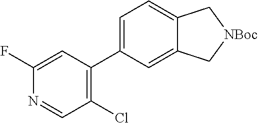 [SMILES: CC(C)(C)OC(=O)N1Cc2ccc(-c3cc(F)ncc3Cl)cc2C1]

Title building block was prepared from intermediate I-4 using Suzuki coupling procedure G and 5-chloro-2-fluoro-4-iodopyridine (100 mg, 0.388 mmol). The crude compound was purified by column chromatography using 0-30% EtOAc in n-hexanes to afford title compound (80 mg, 59.26%). ¹H NMR (400 MHz, Chloroform-d) δ 8.29 (s, 1H), 7.44-7.30 (m, 3H), 6.94 (t, J=2.9 Hz, 1H), 4.76 (s, 2H), 4.73 (s, 2H), 1.53 (s, 9H).

### Preparation of 5-(5-chloro-2-fluoropyridin-4-yl)isoindoline trifluoro acetic Acid Salt

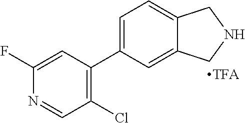 [SMILES: Fc1cc(-c2ccc3c(c2)CNC3)c(Cl)cn1]

Title compound was prepared from precursor (80 mg, 0.22 mmol) using Boc-deprotection procedure A. The crude material was taken to next step as such (80 mg). LC-MS m/z 248.95 [M+H]⁺, retention time=1.26 min (Method A); HPLC: 97.63%, retention time=5.46 min (Method E).

### Preparation of 1-(2-(5-(5-chloro-2-fluoropyridin-4-yl)isoindolin-2-yl)-2-oxoethyl)-1H-1,2,4-triazole-3-carbonitrile (3)

Compound 3 was prepared from intermediate I-3 using acid amide coupling procedure A and precursor (50 mg, 0.20 mmol). The crude compound was purified by column chromatography using 3% MeOH in DCM to afford title compound as off white solid (16 mg, 20.77%). ¹H NMR (300 MHz, Chloroform-d) δ 8.46 (s, 1H), 8.31 (s, 1H), 7.50-7.42 (m, 3H), 6.95 (d, J=2.4 Hz, 1H), 5.17 (s, 2H), 5.04 (s, 2H), 4.94 (s, 2H); LC-MS m/z 380.85 [M−H]⁺, retention time=1.49 min (Method A); HPLC: 95.12%, retention time=7.18 min (Method B).

### Example 4: 1-(2-(5-(3-chloro-2-(trifluoromethyl)phenyl)isoindolin-2-yl)-2-oxoethyl)-1H-1,2,4-triazole-3-carbonitrile

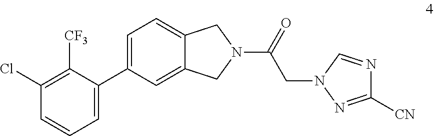 [SMILES: N#Cc1ncn(CC(=O)N2Cc3ccc(-c4cccc(Cl)c4C(F)(F)F)cc3C2)n1]

### Preparation of tert-butyl 5-(3-chloro-2-(trifluoromethyl)phenyl)isoindoline-2-carboxylate

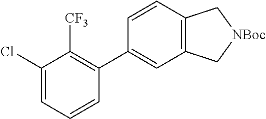 [SMILES: CC(C)(C)OC(=O)N1Cc2ccc(-c3cccc(Cl)c3C(F)(F)F)cc2C1]

Title compound was prepared from 1-bromo-3-chloro-2-(trifluoromethyl)benzene using Suzuki coupling procedure F and boronate ester I-4. Purification with silica gel column chromatography using 0-30% EtOAc in n-hexanes provided product (120 mg, 52.4% yield). ¹H NMR (400 MHz, Chloroform-d) δ 7.58-7.48 (m, 1H), 7.42 (t, J=8.1 Hz, 1H), 7.35-7.19 (m, 2H), 7.20-7.05 (m, 2H), 4.74-4.62 (m, 4H), 1.52 (s, 9H).

### Preparation of 5-(3-chloro-2-(trifluoromethyl)phenyl)isoindoline

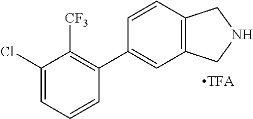 [SMILES: FC(F)(F)c1c(Cl)cccc1-c1ccc2c(c1)CNC2]

Title compound was prepared using Boc-deprotection procedure A. The crude material (100 mg) taken for the next step without purification. LC-MS m/z 297.90 [M+H]⁺, retention time=1.35 min (Method A).

### Preparation of 1-(2-(5-(3-chloro-2-(trifluoromethyl)phenyl)isoindolin-2-yl)-2-oxoethyl)-1H-1,2,4-triazole-3-carbonitrile (4)

Example 4 was prepared using acid-amine coupling procedure A and acid intermediate I-3. Purification with silica gel column chromatography using 30-100% EtOAc in n-hexanes provided 4 (13 mg, 12.0% yield) as grey solid. ¹H NMR (400 MHz, DMSO-d6): δ 8.89 and 8.88 (s, 1H), 7.78 (d, J=7.6 Hz, 1H), 7.72-7.67 (m, 1H), 7.46 (d, J=8.0 Hz, 1H), 7.36-7.30 (m, 2H), 7.25 (d, J=8.0 Hz, 1H), 5.52 (s, 2H), 5.02 and 4.98 (s, 2H), 4.76-4.73 (s, 2H); (NMR shows doubling of protons due to presence of rotamers); LC-MS m/z 431.90 [M+H]⁺, retention time=1.57 min (Method A); HPLC: 97.10%, retention time=4.95 min (Method C).

### Example 5: 1-(2-oxo-2-(5-(2-(2,2,2-trifluoroethoxy)pyridin-3-yl)isoindolin-2-yl)ethyl)-1H-1,2,4-triazole-3-carbonitrile

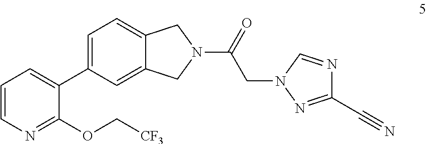 [SMILES: N#Cc1ncn(CC(=O)N2Cc3ccc(-c4cccnc4OCC(F)(F)F)cc3C2)n1]

### Preparation of 3-bromo-2-(2,2,2-trifluoroethoxy)pyridine

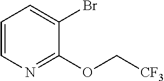 [SMILES: FC(F)(F)COc1ncccc1Br]

To a cooled suspension of NaH (74 mg, 1.56 mmol) in DMF (3 mL) was added trifluoroethanol (156 mg, 1.56 mmol) dropwise at 0° C. and resulting solution was stirred at rt for 1 h. To this added solution of 3-bromo-2-chloropyridine (150 mg, 0.78 mmol) in DMF (1 mL) dropwise and stirred reaction mixture at 55° C. for 48 h. Reaction mixture diluted with EtOAc (25 mL) and washed with water, organic phase extracted with EtOAc (20 mL×2). Combined organic phases dried on sodium sulphate, filtered and evaporated in vacuo. Crude compound was purified by silica gel column chromatography using 0-30% EtOAc in n-hexanes to afford title compound 110 mg (50.0%), Rf=0.55 (30% EtOAc in n-Hexane). ¹H NMR (300 MHz, Chloroform-d) δ 8.08 (dd, J=4.9, 1.6 Hz, 1H), 7.87 (dd, J=7.7, 1.7 Hz, 1H), 6.88 (dd, J=7.7, 4.9 Hz, 1H), 4.81 (q, J=8.4 Hz, 2H).

### Preparation of tert-butyl 5-(2-(2,2,2-trifluoroethoxy)pyridin-3-yl)isoindoline-2-carboxylate

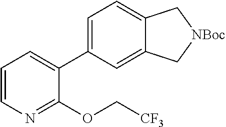 [SMILES: CC(C)(C)OC(=O)N1Cc2ccc(-c3cccnc3OCC(F)(F)F)cc2C1]

Title compound was prepared using Suzuki coupling procedure F and boronate ester I-4. Purification with silica gel column chromatography using 0-50% EtOAc in n-hexanes provided product (90 mg, 58.82% yield). ¹H NMR (400 MHz, Chloroform-d) δ 8.13 (dt, J=4.9, 1.5 Hz, 1H), 7.69 (ddd, J=7.3, 4.2, 1.9 Hz, 1H), 7.52-7.39 (m, 2H), 7.36-7.26 (m, 1H), 7.08 (ddd, J=7.4, 4.9, 1.5 Hz, 1H), 4.82 (qd, J=8.6, 3.1 Hz, 2H), 4.74 (s, 2H), 4.69 (s, 2H), 1.53 (s, 9H).

### Preparation of 5-(2-(2,2,2-trifluoroethoxy)pyridin-3-yl)isoindoline

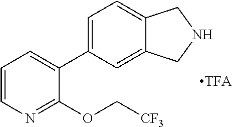 [SMILES: FC(F)(F)COc1ncccc1-c1ccc2c(c1)CNC2]

Title compound was prepared from precursor using Boc-deprotection procedure A. The crude material (90 mg) taken for the next step without purification. LC-MS m/z 295 [M+H]⁺, retention time=1.30 min (Method A).

### Preparation of 1-(2-oxo-2-(5-(2-(2,2,2-trifluoroethoxy)pyridin-3-yl)isoindolin-2-yl)ethyl)-1H-1,2,4-triazole-3-carbonitrile (5)

Compound 5 was prepared using acid-amine coupling procedure A and acid intermediate I-3. Purification with silica gel column chromatography using 20-100% EtOAc in n-hexanes provided 5 (50 mg, 38.16% yield) as Off white solid. ¹H NMR (400 MHz, Chloroform-d): δ 8.46 (s, 1H), 8.18-8.14 (m, 1H), 7.70 (dd, J=7.2, 1.8 Hz, 1H), 7.56-7.50 (m, 2H), 7.39 (m, J=7.5 Hz, 1H), 7.13-7.06 (m, 1H), 5.17 (s, 2H), 5.00 (s, 2H), 4.90 (s, 2H), 4.88-4.77 (m, 2H); LC-MS m/z 429.0 [M+H]⁺, retention time=1.53 min (Method A); HPLC: 98.35%, retention time=7.07 min (Method E).

### Example 6: 1-(2-(5-(6-methyl-2-(trifluoromethyl)pyridin-3-yl)isoindolin-2-yl)-2-oxoethyl)-1H-1,2,4-triazole-3-carbonitrile

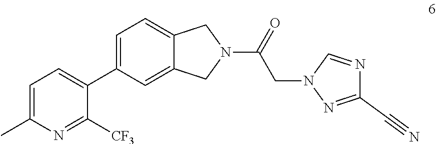 [SMILES: Cc1ccc(-c2ccc3c(c2)CN(C(=O)Cn2cnc(C#N)n2)C3)c(C(F)(F)F)n1]

### Preparation of tert-butyl 5-(6-amino-2-(trifluoromethyl)pyridin-3-yl)isoindoline-2-carboxylate

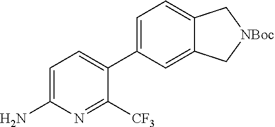 [SMILES: CC(C)(C)OC(=O)N1Cc2ccc(-c3ccc(N)nc3C(F)(F)F)cc2C1]

Title compound was prepared from 5-bromo-6-(trifluoromethyl)pyridin-2-amine using Suzuki coupling procedure F and boronate ester I-4. Purification with silica gel column chromatography using 0-50% EtOAc in n-hexanes provided product (500 mg, 45.4% yield). ¹H NMR (300 MHz, Chloroform-d) δ 7.41 (d, J=8.4 Hz, 1H), 7.35-7.20 (m, 1H), 7.22-7.08 (m, 2H), 6.68 (d, J=8.4 Hz, 1H), 4.73 (s, 2H), 4.69 (s, 2H), 1.52 (s, 9H).

### Preparation of tert-butyl 5-(6-bromo-2-(trifluoromethyl)pyridin-3-yl)isoindoline-2-carboxylate

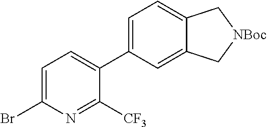 [SMILES: CC(C)(C)OC(=O)N1Cc2ccc(-c3ccc(Br)nc3C(F)(F)F)cc2C1]

To a cooled solution of precursor (500 mg, 1.31 mmol) in dry THF (10 mL) was added CuBr₂ (441 mg, 1.97 mmol) at 0° C. followed by dropwise addition of t-butyl nitrite (0.2 mL, 1.58 mmol) and stirred at 0° C. for 1 h. Water (20 mL) was added to reaction and extracted with EtOAc (50 mL×2), combined organic layer washed with brine solution, dried on sodium sulphate and concentrated in vacuo. Crude compound was purified by column chromatography using 0-30% EtOAc in n-hexanes to afford title compound 300 mg (51.4%), Rf=0.4 (20% EtOAc in n-Hexane); ¹H NMR (400 MHz, Chloroform-d) δ 7.71 (d, J=8.0 Hz, 1H), 7.60-7.48 (m, 1H), 7.35-7.26 (m, 1H), 7.24-7.12 (m, 2H), 4.74 (s, 2H), 4.71 (s, 2H), 1.52 (s, 9H).

### Preparation of tert-butyl 5-(6-methyl-2-(trifluoromethyl)pyridin-3-yl)isoindoline-2-carboxylate

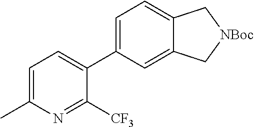 [SMILES: Cc1ccc(-c2ccc3c(c2)CN(C(=O)OC(C)(C)C)C3)c(C(F)(F)F)n1]

To a sealed tube containing precursor mg, 0.67 mmol) in DME (30 mL) was added K₂CO₃ (234 mg, 1.69 mmol), Methyl boronic acid (65 mg, 1.08 mmol) and Pd(dppf)Cl₂.CH₂Cl₂ (10 mol %). The reaction mixture was heated at 100° C. for 12 hours before it was diluted with H₂O and extracted with EtOAc (twice). The combined organic layers were washed with H₂O, then brine, dried over Na₂SO₄, filtered and concentrated under vacuo to give crude material which was purified by silica gel chromatography using 10-15% EtOAc in n-hexanes to afford title compound as pale yellow liquid 0.18 g (70.21%, yield) ¹H NMR (600 MHz, DMSO-d6) δ 7.82 (dd, J=8.2, 2.5 Hz, 1H), 7.67 (d, J=8.0 Hz, 1H), 7.46 (t, J=8.1 Hz, 1H), 7.35 (d, J=6.4 Hz, 1H), 7.28 (d, J=7.8 Hz, 1H), 4.68 (t, J=14.9 Hz, 4H), 2.65 (d, J=1.5 Hz, 3H), 1.51 (s, 9H). LC-MS m/z 379.9 [M+H]⁺, retention time=1.88 min (Method AB).); HPLC: 97.37%, retention time=7.66 min (Method F).

### Preparation of 5-(6-methyl-2-(trifluoromethyl)pyridin-3-yl)isoindoline trifluoroacetic Acid Salt

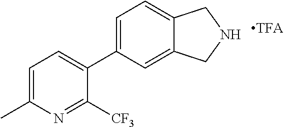 [SMILES: Cc1ccc(-c2ccc3c(c2)CNC3)c(C(F)(F)F)n1]

Title compound was prepared from precursor (0.18 g) using Boc-deprotection procedure A. The crude material taken as such for next step (0.18 g). LC-MS m/z 279.00 [M+H]⁺, retention time=1.28 min (Method A).

### Preparation of 1-(2-(5-(6-methyl-2-(trifluoromethyl)pyridin-3-yl)isoindolin-2-yl)-2-oxoethyl)-1H-1,2,4-triazole-3-carbonitrile (6)

Example 6 was prepared from intermediate I-3 using acid amide coupling procedure A. Purification with reversed-phase HPLC (Method B) provided 6 (40 mg, 20.30% yield) as brown solid. ¹H NMR (600 MHz, DMSO-d6): δ 8.89 and 8.88 (s, 1H), 7.79 (t, J=7.5 Hz, 1H), 7.64 (dd, J=7.2 Hz and 3.6 Hz, 1H), 7.49 (d, J=7.2 Hz, 1H), 7.37 (s, 1H), 7.29 (d, J=7.2 Hz, 1H), 5.52 (s, 2H), 5.02 and 5.01 (s, 2H), 4.76 and 4.74 (s, 2H), 2.60 (s, 3H). (NMR shows doubling of protons due to presence of rotamers); LC-MS m/z 413.00 [M+H]⁺, retention time=1.50 min (Method A); HPLC: 99.02%, retention time=5.87 min (Method F).

### Example 7: 1-(2-(5-(5-fluoro-2-(trifluoromethyl)pyridin-3-yl)isoindolin-2-yl)-2-oxoethyl)-1H-1,2,4-triazole-3-carbonitrile

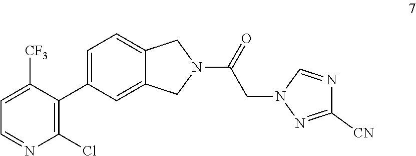 [SMILES: N#Cc1ncn(CC(=O)N2Cc3ccc(-c4c(C(F)(F)F)ccnc4Cl)cc3C2)n1]

### Preparation of tert-butyl 5-(2-chloro-4-(trifluoromethyl)pyridin-3-yl)isoindoline-2-carboxylate

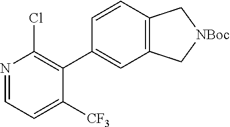 [SMILES: CC(C)(C)OC(=O)N1Cc2ccc(-c3c(C(F)(F)F)ccnc3Cl)cc2C1]

Title compound was prepared from 3-bromo-2-chloro-4-(trifluoromethyl)pyridine using Suzuki coupling procedure F and boronate ester I-4. Purification with silica gel column chromatography using 0-20% EtOAc in n-hexanes provided product (120 mg, 51.9% yield). ¹H NMR (300 MHz, Chloroform-d) δ 8.60 (d, J=5.1 Hz, 1H), 7.61 (d, J=5.1 Hz, 1H), 7.40-7.30 (m, 1H), 7.21-7.05 (m, 2H), 4.84-4.67 (m, 4H), 1.52 (s, 9H).

### Preparation of 5-(2-chloro-4-(trifluoromethyl)pyridin-3-yl)isoindoline

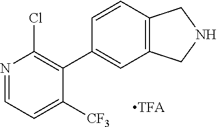 [SMILES: FC(F)(F)c1ccnc(Cl)c1-c1ccc2c(c1)CNC2]

Title compound was prepared using Boc-deprotection procedure A. The crude material (100 mg) taken for the next step without purification. LC-MS m/z 298.90 [M+H]⁺, retention time=1.28 min (Method A).

### Preparation of 1-(2-(5-(2-fluoro-4-(trifluoromethyl)pyridin-3-yl)isoindolin-2-yl)-2-oxoethyl)-1H-1,2,4-triazole-3-carbonitrile (7)

Example 7 was prepared using acid-amine coupling procedure A and acid intermediate I-3. Purification with silica gel column chromatography using 20-100% EtOAc in n-hexanes provided 7 (40 mg, 28.1% yield) as white solid. ¹H NMR (400 MHz, DMSO-d6): δ 8.90 and 8.88 (s, 1H), 8.75 (d, J=5.2 Hz, 1H), 7.95-7.90 (m, 1H), 7.52 (dd, J=7.2 Hz and 3.2 Hz, 1H), 7.39 (d, J=9.2 Hz, 1H), 7.31-7.25 (m, 1H), 5.52 (s, 2H), 5.04 and 5.02 (s, 2H), 4.78 and 4.73 (s, 2H) (NMR shows doubling of protons due to presence of rotamers); LC-MS m/z 433.2 [M+H]⁺, retention time=1.48 min (Method E); HPLC: 97.85%, retention time=5.82 min (Method F).

### Example 8: 1-(2-(3-(2,4-dichlorophenyl)-5,7-dihydro-6H-pyrrolo[3,4-b]pyridin-6-yl)-2-oxoethyl)-1H-1,2,4-triazole-3-carbonitrile

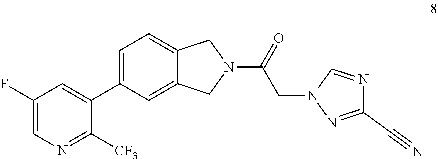 [SMILES: N#Cc1ncn(CC(=O)N2Cc3ccc(-c4cc(F)cnc4C(F)(F)F)cc3C2)n1]

### Preparation of 3-bromo-5-fluoro-2-iodopyridine

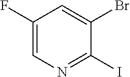

2,3-dibromo-5-fluoropyridine (12.5 g, 49.042 mmol) and Sodium iodide (21.90 g, 147.128 mmol) in Acetonitrile (125 mL) were stirred in a sealed tube and Chloro trimethylsilane (5.32 g, 49.042 mmol) was added dropwise under argon atmosphere and the reaction mixture was stirred at 75° C. for 1 h. Reaction mixture was quenched with Aq. Ammonia (10 ml), extracted with Diethyl ether (500 mL×2) twice. The combined organic layers were washed with water (100 mL), brine (100 mL), dried over sodium sulphate and concentrated under vacuo. Crude compound was purified by Combiflash chromatography using 40 g Column and 2-3% EtOAc in n-Hexane to afford the title compound as a Light brown solid 7.5 g (50.66%). Rf=0.4 (100% n-Hexane); ¹H NMR (300 MHz, CDCl₃): δ 8.27 (d, J=2.7 Hz, 1H), 7.64-7.61 (m, 1H).

### Preparation of 3-bromo-5-fluoro-2-(trifluoromethyl) pyridine

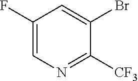 [SMILES: Fc1cnc(C(F)(F)F)c(Br)c1]

3-bromo-5-fluoro-2-iodopyridine (5 g, 16.56 mmol), Copper iodide (22.08 g, 115.94 mmol) and dry DMF (100 mL) were stirred in a sealed tube and Methyl 2,2-difluoro-2-(fluorosulfonyl) acetate (22.27 g, 115.94 mmol) was added dropwise under argon atmosphere and the reaction mixture was stirred at 70° C. for 16 h. Reaction mixture was filtered and filterate was extracted with Methyl t-Butyl ether (500 mL×2) twice. The combined organic layers were washed with water (100 mL), brine (100 mL), dried over sodium sulphate and concentrated under vacuo. Crude compound was purified by Combiflash chromatography using 40 g Column and 100% n-Hexane to afford the title compound as a Light brown solid 4 g (99%). Rf=0.4 (100% n-Hexane); ¹H NMR (300 MHz, CDCl₃): δ 8.50 (d, J=2.4 Hz, 1H), 7.85-7.82 (m, 1H).

### Preparation of tert-butyl 5-(4,4,5,5-tetramethyl-1,3,2-dioxaborolan-2-yl) isoindoline-2-carboxylate

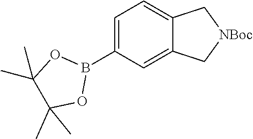 [SMILES: CC(C)(C)OC(=O)N1Cc2ccc(B3OC(C)(C)C(C)(C)O3)cc2C1]

Tert-butyl-5-bromisoindoline-2-carboxylate (20.0 g, 67.09 mmol) was dissolved in Dioxane (200 mL) and the solution was purged with Argon gas for 10 min. Bispinacalato diborane (34.07 g, 134.18 mmol), KOAc (26.34 g, 268.36 mmol) and Pd(PPh₃)₄ (7.75 g, 6.70 mmol) were added and the reaction mixture was stirred at 80° C. for 12 h. Reaction mixture was diluted with water, extracted with EtOAc (1 Ltr.×2) twice. The combined organic layers were washed with water (500 mL), brine (300 mL), dried over sodium sulphate and concentrated under vacuo. Crude compound was purified by Combiflash chromatography using 40 g Column and 10% EtOAc in n-Hexane to afford title compound as a White solid 23.0 g (99.30%). Rf=0.50 (10% EtOAc in n-Hexane); ¹H NMR (300 MHz, CDCl₃): δ 7.72-7.65 (m, 2H), 7.31-7.20 (m, 1H), 4.70-4.60 (m, 4H), 1.40 (s, 9H), 1.37 (s, 12H).

### Preparation of tert-butyl 5-(5-fluoro-2-(trifluoromethyl) pyridin-3-yl) isoindoline-2-carboxylate

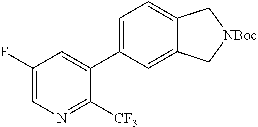 [SMILES: CC(C)(C)OC(=O)N1Cc2ccc(-c3cc(F)cnc3C(F)(F)F)cc2C1]

Tert-butyl 5-(4,4,5,5-tetramethyl-1,3,2-dioxaborolan-2-yl) isoindoline-2-carboxylate (5.6 g, 16.39 mmol) was dissolved in Dioxane:H₂O (8:2) (100 mL), purged with Argon gas for 10 min., K₃PO₄ (8.67 g, 40.98 mmol), 3-bromo-5-fluoro-2-(trifluoromethyl)pyridine (4.0 g, 16.39 mmol) and Pd(dppf)Cl₂.DCM (1.34 g, 1.63 mmol) were added and the reaction mixture was stirred at 100° C. for 3 h. Reaction mixture was diluted with water, extracted with EtOAc (500 mL×2) twice. The combined organic layers were washed with water (300 mL), brine (300 mL), dried over sodium sulphate and concentrated in vacuo. Crude compound was purified by Combiflash chromatography using 40 g Column and 25% EtOAc in n-Hexane to afford title compound as a brownish semisolid 3.0 g (47.86%). Rf=0.50 (20% EtOAc in n-Hexane); ¹H NMR (300 MHz, CDCl₃): δ 8.56 (d, J=2.4 Hz, 1H), 7.50-7.32 (m, 2H), 7.32-7.15 (m, 2H), 4.75 (s, 2H), 4.71 (s, 2H), 1.52 (s, 9H). LC-MS: [M-Boc]+1=282.8.

### Preparation of 5-(5-fluoro-2-(trifluoromethyl)pyridin-3-yl)isoindoline trifluoroacetic Acid Salt

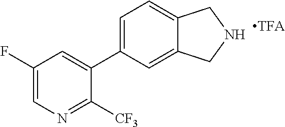 [SMILES: Fc1cnc(C(F)(F)F)c(-c2ccc3c(c2)CNC3)c1]

Tert-butyl 5-(5-fluoro-2-(trifluoromethyl)pyridin-3-yl)isoindoline-2-carboxylate (3 g, 382.36 mmol) was dissolved in DCM (15 mL) and trifluoroacetic acid (15 mL) was added at 0° C. The reaction mixture was stirred for 1 h and concentrated under vacuo. Crude compound was purified by 10% Diethyl ether in n-Pentane washings to afford title compound as a Wheatish solid 3.0 g (Crude). Rf=0.10 (10% MeOH in DCM); LC-MS: [M-TFA]+1=282.9, HPLC: 96.87%, RT: 5.676 min.

### Preparation of 1-(2-(5-(5-fluoro-2-(trifluoromethyl) pyridin-3-yl) isoindolin-2-yl)-2-oxoethyl)-1H-1,2,4-triazole-3-carbonitrile

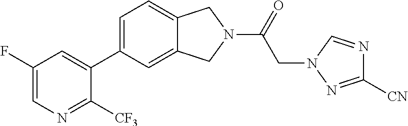 [SMILES: N#Cc1ncn(CC(=O)N2Cc3ccc(-c4cc(F)cnc4C(F)(F)F)cc3C2)n1]

To a stirred solution of unprotected isoindoline derivative (3.0 g, 7.57 mmol) and 2-(3-cyano-1H-1,2,4-triazol-1-yl)acetic acid (7.34 g, 48.34 mmol) in DCM (45 mL) were added DIPEA (4.89 g, 37.85 mmol), Propyl phosphonic anhydride (T3P) (50% EtOAc solution) (6.26 mL, 9.84 mmol) at 0° C. and stirred at RT for 3 h. Reaction mixture was diluted with water, extracted with DCM (500 mL×2) twice. The combined organic layers were washed with water (200 mL), brine (150 mL), dried over sodium sulphate and concentrated in vacuo. Crude compound was purified by Combiflash chromatography using 40 g Column and 70% EtOAc in n-Hexane to afford the title compound as a White solid 2.5 g (79.31%). Rf=0.4 (100% EtOAc in hexane); ¹H NMR (600 MHz, DMSO): 8.89 (dd, J=6.0 Hz and 1.8 Hz, 1H), 8.84-8.82 (m, 1H), 7.99 (t, J=8.6 Hz, 1H), 7.54-7.51 (m, 1H), 7.45 (s, 1H), 7.36 (d, J=7.8 Hz, 1H), 5.53 (s, 2H), 5.04 and 5.02 (s, 2H), 4.77 and 4.75 (s, 2H); LC-MS: 416.7 (M+H), HPLC: 99.43%, RT: 7.173 min.

### Example 9: 1-(2-(5-(5-fluoro-4-(trifluoromethyl)pyridin-3-yl)isoindolin-2-yl)-2-oxoethyl)-1H-1,2,4-triazole-3-carbonitrile

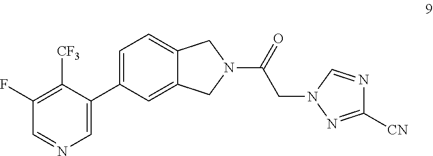 [SMILES: N#Cc1ncn(CC(=O)N2Cc3ccc(-c4cncc(F)c4C(F)(F)F)cc3C2)n1]

### Preparation of tert-butyl 5-(5-fluoro-4-(trifluoromethyl)pyridin-3-yl)isoindoline-2-carboxylate

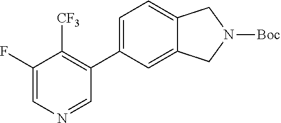 [SMILES: CC(C)(C)OC(=O)N1Cc2ccc(-c3cncc(F)c3C(F)(F)F)cc2C1]

Title compound was prepared from intermediate I-4 using Suzuki coupling procedure F and 3-bromo-5-fluoro-4-(trifluoromethyl) pyridine. The crude compound was purified by column chromatography using 10-15% EtOAc in n-hexanes to afford title compound as Colourless sticky mass 0.04 g (51.28%, yield) ¹H NMR (300 MHz, Chloroform-d) δ 8.65 (s, 1H), 8.43 (s, 1H), 7.42-7.11 (m, 3H), 4.76 (s, 2H), 4.72 (s, 2H), 1.53 (s, 9H); LC-MS m/z 381.90 [M−H]⁺, retention time=1.68 min (Method A).

### Preparation of 5-(5-fluoro-4-(trifluoromethyl)pyridin-3-yl)isoindoline trifluoroacetic Acid Salt

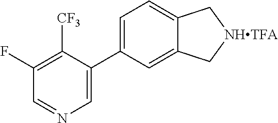

Title compound was prepared using TFA procedure A. The crude compound was purified by triturating in pentane to afford title compound as light brown sticky mass 0.04 g.

### Preparation of 1-(2-(5-(5-fluoro-4-(trifluoromethyl)pyridin-3-yl)isoindolin-2-yl)-2-oxoethyl)-1H-1,2,4-triazole-3-carbonitrile (9)

Compound 9 was prepared from intermediate I-3 using acid amide coupling procedure. Purification with reversed-phase HPLC (Method C) provided 9 (10 mg, 23.81% yield) as white solid. ¹H NMR (300 MHz, Chloroform-d) δ 8.68 (s, 1H), 8.46 (s, 1H), 8.43 (s, 1H), 7.46-7.26 (m, 3H), 5.17 (s, 2H), 5.04 (s, 2H), 4.93 (s, 2H); LC-MS m/z 416.95 [M+H]⁺, retention time=1.49 min (Method A); HPLC: 97.81%, retention time=5.76 min (Method F).

### Example 10: 1-(2-(5-(2-chloro-4,6-difluorophenyl)isoindolin-2-yl)-2-oxoethyl)-1H-1,2,4-triazole-3-carbonitrile

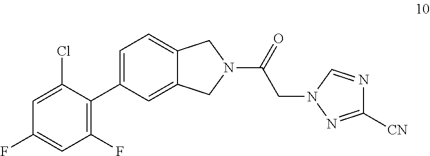 [SMILES: N#Cc1ncn(CC(=O)N2Cc3ccc(-c4c(F)cc(F)cc4Cl)cc3C2)n1]

### Preparation of tert-butyl 5-(2-chloro-4,6-difluorophenyl)isoindoline-2-carboxylate

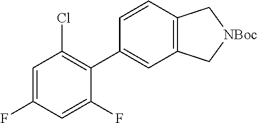 [SMILES: CC(C)(C)OC(=O)N1Cc2ccc(-c3c(F)cc(F)cc3Cl)cc2C1]

Title compound was prepared from 2-bromo-1-chloro-3,5-difluorobenzene using Suzuki coupling procedure F and boronate ester I-4. Purification with silica gel column chromatography using 0-20% EtOAc in n-hexanes provided product (100 mg, 47.84% yield). ¹H NMR (400 MHz, Chloroform-d) δ 7.40-7.31 (m, 1H), 7.24-7.16 (m, 2H), 7.08 (dt, J=8.3, 2.1 Hz, 1H), 6.86 (td, J=8.8, 8.7, 2.6 Hz, 1H), 4.75 (s, 2H), 4.71 (s, 2H), 1.52 (s, 9H).

### Preparation of 5-(2-chloro-4,6-difluorophenyl)isoindoline

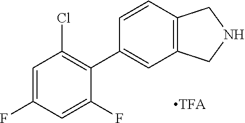 [SMILES: Fc1cc(F)c(-c2ccc3c(c2)CNC3)c(Cl)c1]

Title compound was prepared using Boc-deprotection procedure A. The crude material (100 mg) taken for the next step without purification. LC-MS m/z 265.90 [M+H]⁺, retention time=1.31 min (Method A).

### Preparation of 1-(2-(5-(2-chloro-4,6-difluorophenyl)isoindolin-2-yl)-2-oxoethyl)-1H-1,2,4-triazole-3-carbonitrile (10)

Compound 10 was prepared using acid-amine coupling procedure A and acid I-3. Purification with silica gel column chromatography using 20-90% EtOAc in n-hexanes provided 10 (11 mg, 10% yield) as white solid. ¹H NMR (300 MHz, DMSO-d6): δ 8.90 and 8.89 (s, 1H), 7.56-7.46 (m, 3H), 7.39 (d, J=6.0 Hz, 1H), 7.30 (d, J=7.2 Hz, 1H), 5.52 (s, 2H), 5.02 and 5.01 (s, 2H), 4.76 and 4.74 (s, 2H); (NMR shows doubling of protons due to presence of rotamers); LC-MS m/z 399.80 [M+H]⁺, retention time=2.30 min (Method H); HPLC: 98.51%, retention time=6.38 min (Method F).

### Example 11: 1-(2-(5-(4-fluoro-2-(pyrrolidin-1-yl)phenyl)isoindolin-2-yl)-2-oxoethyl)-1H-1,2,4-triazole-3-carbonitrile

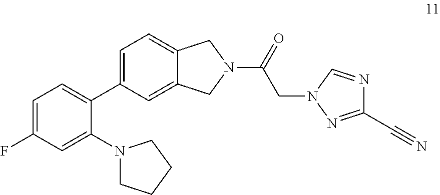 [SMILES: N#Cc1ncn(CC(=O)N2Cc3ccc(-c4ccc(F)cc4N4CCCC4)cc3C2)n1]

### Preparation of 1-(2-bromo-5-fluorophenyl)pyrrolidine

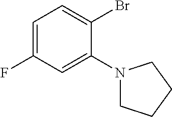 [SMILES: Fc1ccc(Br)c(N2CCCC2)c1]

To a solution of 2-bromo-5-fluoroaniline (0.5 g, 2.63 mmol) in toluene (5.0 mL) was added diisopropyl ethyl amine (0.85 g, 6.57 mmol) drop wise at room temperature, then 1,4-dibromo butane (0.85 g, 3.94 mmol) was added drop wise at room temperature and stirred at 115° C. for 16 h. The reaction mixture diluted with water and extracted with ethylacetate (20 mL×2). The combined organic layer was dried over sodium sulphate and concentrated in vacuo. The crude compound was purified by Column chromatography using 0-5% EtOAc in n-Hexane to afford title compound as colourless liquid (0.1 g, 15.57%) Rf=0.8 (10% EtOAc in n-Hexane); ¹H NMR (600 MHz, Chloroform-d) δ 7.43-7.37 (m, 1H), 6.57 (dd, J=11.8, 2.9 Hz, 1H), 6.46-6.40 (m, 1H), 3.41-3.35 (m, 4H), 1.98-1.90 (m, 4H).

### Preparation of tert-butyl 5-(4-fluoro-2-(pyrrolidin-1-yl) phenyl)isoindoline-2-carboxylate

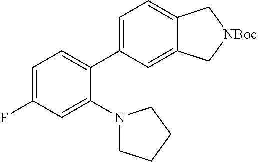 [SMILES: CC(C)(C)OC(=O)N1Cc2ccc(-c3ccc(F)cc3N3CCCC3)cc2C1]

Title compound was prepared from intermediate I-4 using Suzuki coupling procedure K and 1-(2-bromo-5-fluorophenyl)pyrrolidine. The crude compound was purified by column chromatography using 10% EtOAc in n-hexanes to afford title compound as colourless liquid 0.06 g (38.35%, yield). ¹H NMR (400 MHz, Chloroform-d) δ 7.30-7.14 (m, 3H), 7.03 (ddd, J=8.3, 7.0, 1.4 Hz, 1H), 6.57-6.45 (m, 2H), 4.69 (s, 2H), 4.65 (s, 2H), 2.90-2.81 (m, 4H), 1.78-1.70 (m, 4H), 1.51 (s, 9H); LC-MS m/z 383.4 [M+H]⁺, retention time=2.14 min (Method B); HPLC: 85.72%, retention time=9.39 min (Method F).

### Preparation of 5-(4-fluoro-2-(pyrrolidin-1-yl)phenyl)isoindoline trifluoroacetic Acid Salt

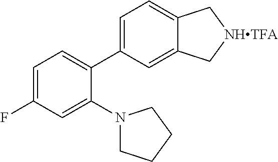

Title compound was prepared from precursor (0.23 g) using Boc-deprotection procedure A. The crude material taken as such for next step (0.25 g). LC-MS m/z 283.3 [M+H]⁺, retention time=0.38 min (Method B); HPLC: 85.79%, retention time=6.03 min (Method O).

### Preparation of 1-(2-(5-(4-fluoro-2-(pyrrolidin-1-yl)phenyl)isoindolin-2-yl)-2-oxoethyl)-1H-1,2,4-triazole-3-carbonitrile (11)

Compound 11 was prepared from intermediate I-3 using acid amide coupling procedure A. Purification with reversed-phase HPLC (Method A) provided 11 (30 mg, 11.43% yield) as white solid. ¹H NMR (400 MHz, Chloroform-d) δ 8.46 (s, 1H), 7.40-7.25 (m, 3H), 7.06-7.02 (m, 1H), 6.60-6.50 (m, 2H), 5.16 (s, 2H), 4.98 (s, 2H), 4.88 (s, 2H), 2.92-2.85 (m, 4H), 1.80-1.73 (m, 4H); LC-MS m/z 417.3 [M+H]⁺, retention time=1.79 min (Method B); HPLC: 99.72%, retention time=5.26 min (Method C).

### Example 12: 1-(2-(5-(2-fluoro-6-(trifluoromethyl)phenyl)isoindolin-2-yl)-2-oxoethyl)-1H-1,2,4-triazole-3-carbonitrile

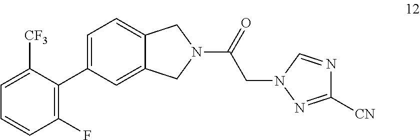 [SMILES: N#Cc1ncn(CC(=O)N2Cc3ccc(-c4c(F)cccc4C(F)(F)F)cc3C2)n1]

### Preparation of tert-butyl 5-(2-fluoro-6-(trifluoromethyl)phenyl)isoindoline-2-carboxylate

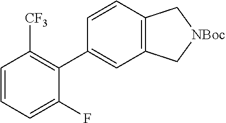 [SMILES: CC(C)(C)OC(=O)N1Cc2ccc(-c3c(F)cccc3C(F)(F)F)cc2C1]

Title compound was prepared from 2-bromo-1-fluoro-3-(trifluoromethyl)benzene using Suzuki coupling procedure F and boronate ester I-4. Purification with silica gel column chromatography using 0-20% EtOAc in n-hexanes provided product (130 mg, 55.1% yield). ¹H NMR (300 MHz, Chloroform-d) δ 7.57 (d, J=7.6 Hz, 1H), 7.55-7.40 (m, 1H), 7.40-7.28 (m, 2H), 7.22-7.13 (m, 2H), 4.84-4.61 (m, 4H), 1.52 (s, 9H).

### Preparation of 5-(2-fluoro-6-(trifluoromethyl)phenyl)isoindoline

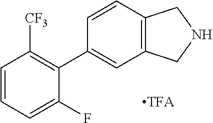 [SMILES: Fc1cccc(C(F)(F)F)c1-c1ccc2c(c1)CNC2]

Title compound was prepared from precursor using Boc-deprotection procedure A. The crude material (130 mg) taken for the next step without purification.

### Preparation of 1-(2-(5-(2-fluoro-6-(trifluoromethyl)phenyl)isoindolin-2-yl)-2-oxoethyl)-1H-1,2,4-triazole-3-carbonitrile (12)

Compound 12 was prepared using acid-amine coupling procedure A and acid I-3. Purification with silica gel column chromatography using 20-90% EtOAc in n-hexanes provided 12 (30 mg, 21.1% yield) as off white solid. ¹H NMR (400 MHz, DMSO-d6): δ 8.90 and 8.89 (s, 1H), 7.78-7.65 (m, 3H), 7.50 (dd, J=8.0, 2.4 Hz, 1H), 7.37 (d, J=5.2 Hz, 1H), 7.27 (d, J=7.2 Hz, 1H), 5.52 and 5.51 (s, 2H), 5.03 and 5.01 (s, 2H), 4.78 and 4.73 (s, 2H); (NMR shows doubling of protons due to presence of rotamers); LC-MS m/z 415.90 [M+H]⁺, retention time=1.53 min (Method A); HPLC: 98.45%, retention time=4.49 min (Method C).

### Example 13: 1-(2-(5-(2-methylpyridin-3-yl)isoindolin-2-yl)-2-oxoethyl)-1H-1,2,4-triazole-3-carbonitrile

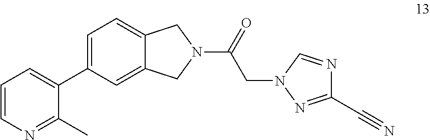 [SMILES: Cc1ncccc1-c1ccc2c(c1)CN(C(=O)Cn1cnc(C#N)n1)C2]

### Preparation of tert-butyl 5-(2-methylpyridin-3-yl)isoindoline-2-carboxylate

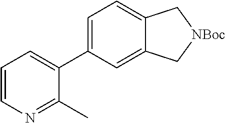 [SMILES: Cc1ncccc1-c1ccc2c(c1)CN(C(=O)OC(C)(C)C)C2]

Title compound was prepared from 3-bromo-2-methylpyridine using Suzuki coupling procedure F and boronate ester I-4. Purification with silica gel column chromatography using 0-50% EtOAc in n-hexanes provided product (120 mg, 66.67% yield). ¹H NMR (300 MHz, Chloroform-d) δ 8.50 (dd, J=4.9, 1.7 Hz, 1H), 7.50 (dt, J=7.7, 1.5, 1.5 Hz, 1H), 7.39-7.07 (m, 4H), 4.75 (s, 2H), 4.71 (s, 2H), 2.50 (s, 3H), 1.53 (s, 9H); LC-MS m/z 311.05 [M+H]⁺, retention time=1.33 min (Method A).

### Preparation of 5-(2-methylpyridin-3-yl)isoindoline

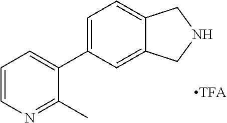 [SMILES: Cc1ncccc1-c1ccc2c(c1)CNC2]

Title compound was prepared using Boc-deprotection procedure A. The crude material (90 mg) taken for the next step without purification. LC-MS m/z 211.0 [M+H]⁺, retention time=0.10 min (Method A).

### Preparation of 1-(2-(5-(2-methylpyridin-3-yl)isoindolin-2-yl)-2-oxoethyl)-1H-1,2,4-triazole-3-carbonitrile (13)

Compound 13 was prepared using acid-amine coupling procedure A and intermediate acid I-3. Purification with silica gel column chromatography using 20-100% EtOAc in n-hexanes provided 13 (30 mg, 18.4% yield) as white solid. ¹H NMR (300 MHz, Chloroform-d): δ 8.55-8.52 (m, 1H), 8.47 (s, 1H), 7.52 (dd, J=7.5 and 1.2 Hz, 1H), 7.45-7.38 (m, 1H), 7.35-7.19 (m, 3H), 5.18 and 5.17 (s, 2H), 5.02 (s, 2H), 4.92 (s, 2H), 2.50 (s, 3H); LC-MS m/z 345.0 [M+H]⁺, retention time=1.24 min (Method A); HPLC: 97.06%, retention time=5.24 min (Method B).

### Example 14: 1-(2-(5-(6-methoxy-2-(trifluoromethyl)pyridin-3-yl)isoindolin-2-yl)-2-oxoethyl)-1H-1,2,4-triazole-3-carbonitrile

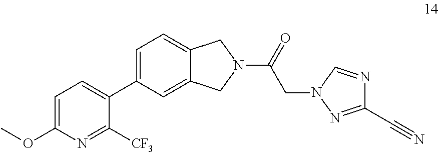 [SMILES: COc1ccc(-c2ccc3c(c2)CN(C(=O)Cn2cnc(C#N)n2)C3)c(C(F)(F)F)n1]

### Preparation of tert-butyl 5-(6-amino-2-(trifluoromethyl)pyridin-3-yl)isoindoline-2-carboxylate

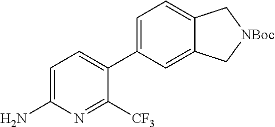 [SMILES: CC(C)(C)OC(=O)N1Cc2ccc(-c3ccc(N)nc3C(F)(F)F)cc2C1]

Title compound was prepared from 5-bromo-6-(trifluoromethyl)pyridin-2-amine using Suzuki coupling procedure F and boronate ester I-4. Purification with silica gel column chromatography using 0-50% EtOAc in n-hexanes provided product (500 mg, 45.4% yield). ¹H NMR (300 MHz, Chloroform-d) δ 7.41 (d, J=8.4 Hz, 1H), 7.35-7.20 (m, 1H), 7.22-7.08 (m, 2H), 6.68 (d, J=8.4 Hz, 1H), 4.73 (s, 2H), 4.69 (s, 2H), 1.52 (s, 9H).

### Preparation of tert-butyl 5-(6-bromo-2-(trifluoromethyl)pyridin-3-yl)isoindoline-2-carboxylate

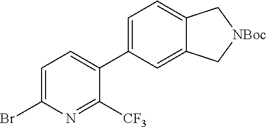 [SMILES: CC(C)(C)OC(=O)N1Cc2ccc(-c3ccc(Br)nc3C(F)(F)F)cc2C1]

To a cooled solution of precursor (500 mg, 1.31 mmol) in dry THF (10 mL) was added CuBr₂ (441 mg, 1.97 mmol) at 0° C. followed by dropwise addition of t-butyl nitrite (0.2 mL, 1.58 mmol) and stirred at 0° C. for 1 h. Water (20 mL) was added to reaction and extracted with EtOAc (50 mL×2), combined organic layer washed with brine solution, dried on sodium sulphate and concentrated in vacuo. Crude compound was purified by column chromatography using 0-30% EtOAc in n-hexanes to afford title compound 300 mg (51.4%), Rf=0.4 (20% EtOAc in n-Hexane); ¹H NMR (400 MHz, Chloroform-d) δ 7.71 (d, J=8.0 Hz, 1H), 7.60-7.48 (m, 1H), 7.35 □7.26 (m, 1H), 7.24-7.12 (m, 2H), 4.74 (s, 2H), 4.71 (s, 2H), 1.52 (s, 9H).

### Preparation of tert-butyl 5-(6-methoxy-2-(trifluoromethyl)pyridin-3-yl)isoindoline-2-carboxylate

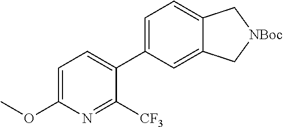 [SMILES: COc1ccc(-c2ccc3c(c2)CN(C(=O)OC(C)(C)C)C3)c(C(F)(F)F)n1]

To the solution of precursor (300 mg, 0.67 mmol) in Methanol (2 mL) was added 25% NaOMe solution (5 mL) at 0° C. and stirred reaction mixture for 10 h at rt. Ice solution (10 mL) was added slowly to reaction and extracted with EtOAc (20 mL×2), combined organic phases dried over sodium sulphate and concentrated in vacuo. Crude compound was purified by column chromatography using 0-30% EtOAc in n-hexanes to afford title compound 150 mg (57.6%), Rf=0.55 (30% EtOAc in n-Hexane). ¹H NMR (400 MHz, Chloroform-d) δ 7.54 (d, J=8.4 Hz, 1H), 7.31 (d, J=7.8 Hz, 1H), 7.23-7.12 (m, 2H), 6.94 (d, J=8.5 Hz, 1H), 4.87-4.58 (m, 4H), 1.52 (s, 9H).

### Preparation of 5-(6-methoxy-2-(trifluoromethyl)pyridin-3-yl)isoindoline

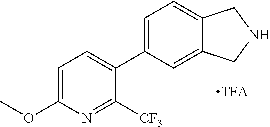 [SMILES: COc1ccc(-c2ccc3c(c2)CNC3)c(C(F)(F)F)n1]

Title compound was prepared using Boc-deprotection procedure A. The crude material (150 mg) taken for the next step without purification. LC-MS m/z 295.0 [M+H]⁺, retention time=1.30 min (Method A).

### Preparation of 1-(2-(5-(6-methoxy-2-(trifluoromethyl)pyridin-3-yl)isoindolin-2-yl)-2-oxoethyl)-1H-1,2,4-triazole-3-carbonitrile (14)

Compound 14 was prepared using acid-amine coupling procedure A and acid intermediate I-3. Purification with silica gel column chromatography using 20-100% EtOAc in n-hexanes provided 14 (43 mg, 26.3% yield) as off white solid. ¹H NMR (400 MHz, Chloroform-d): δ 8.46 (s, 1H), 7.54 (d, J=8.4 Hz, 1H), 7.40-7.35 (m, 1H), 7.40-7.23 (m, 2H), 6.96 (dd, J=8.0 Hz and 3.2 Hz, 1H), 5.17 and 5.16 (s, 2H), 5.01 and 5.00 (s, 2H), 4.91 and 4.90 (s, 2H), 4.03 and 4.02 (s, 3H) (NMR shows doubling of protons due to presence of rotamers); LC-MS m/z 428.95 [M+H]⁺, retention time=1.55 min (Method A). HPLC: 99.33%, retention time=4.20 min (Method D).

### Example 15: 1-(2-(5-(2-chloro-3,6-difluorophenyl)isoindolin-2-yl)-2-oxoethyl)-1H-1,2,4-triazole-3-carbonitrile

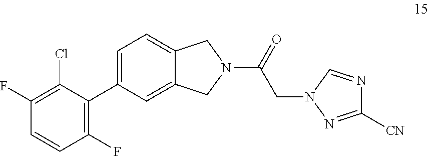 [SMILES: N#Cc1ncn(CC(=O)N2Cc3ccc(-c4c(F)ccc(F)c4Cl)cc3C2)n1]

### Preparation of tert-butyl 5-(2-chloro-3,6-difluorophenyl)isoindoline-2-carboxylate

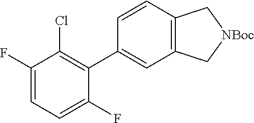 [SMILES: CC(C)(C)OC(=O)N1Cc2ccc(-c3c(F)ccc(F)c3Cl)cc2C1]

Title compound was prepared from 2-bromo-3-chloro-1,4-difluorobenzene using Suzuki coupling procedure F and boronate ester I-4. Purification with silica gel column chromatography using 0-20% EtOAc in n-hexanes provided 2 (120 mg, 49.8% yield). ¹H NMR (300 MHz, Chloroform-d) δ 7.42-7.32 (m, 1H), 7.25-7.01 (m, 4H), 4.87-4.57 (m, 4H), 1.52 (s, 9H).

### Preparation of 5-(2-chloro-3,6-difluorophenyl)isoindoline

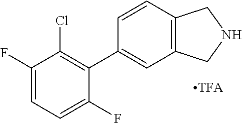 [SMILES: Fc1ccc(F)c(-c2ccc3c(c2)CNC3)c1Cl]

Title compound was prepared Boc-deprotection procedure A. The crude material (100 mg) taken for the next step without purification. LC-MS m/z 265.90 [M+H]⁺, retention time=1.30 min (Method A)

### Preparation of 1-(2-(5-(2-chloro-3,6-difluorophenyl)isoindolin-2-yl)-2-oxoethyl)-1H-1,2,4-triazole-3-carbonitrile (15)

Compound 15 was prepared using acid-amine coupling procedure A and acid intermediate I-3. Purification with silica gel column chromatography using 30-100% EtOAc in n-hexanes provided 15 (30 mg, 27.5% yield) as white solid. ¹H NMR (400 MHz, DMSO-d6): δ 8.90 and 8.89 (s, 1H), 7.60-7.52 (m, 2H), 7.49-7.42 (m, 2H), 7.34 (d, J=7.6 Hz, 1H), 5.53 and 5.52 (s, 2H), 5.03 and 5.02 (s, 2H), 4.77 and 4.75 (s, 2H); (NMR shows doubling of protons due to presence of rotamers); LC-MS m/z 399.90 [M+H]⁺, retention time=1.52 min (Method A); HPLC: 95.03%, retention time=11.76 min (Method A).

### Example 16: 1-(2-(5-(3-fluoro-5-(trifluoromethyl)pyridin-4-yl)isoindolin-2-yl)-2-oxoethyl)-1H-1,2,4-triazole-3-carbonitrile

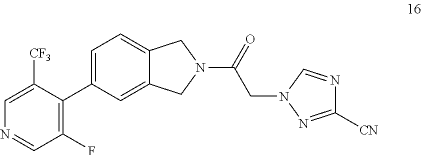 [SMILES: N#Cc1ncn(CC(=O)N2Cc3ccc(-c4c(F)cncc4C(F)(F)F)cc3C2)n1]

### Preparation of 3-fluoro-4-iodo-5-(trifluoromethyl)pyridine

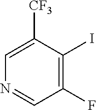 [SMILES: Fc1cncc(C(F)(F)F)c1I]

To a cooled solution of 3-fluoro-5-(trifluoromethyl)pyridine (0.3 g, 1.81 mmol) in anhydrous THF (10 mL) was added LDA (2M in THF, 1.1 mL, 2.18 mmol) dropwise over 5 min and the resulting solution was stirred for 1 h at −78° C. Iodine (0.46 g, 1.81 mmol) in anhydrous THF (2 mL) was then added dropwise over 5 min at −78° C. and stirred for an hour at same temperature. The reaction mixture was then quenched with saturated aq.NH₄Cl solution (5 mL). Crude was extracted with EtOAc (10 mL×2) and combined organic phases were washed with Na₂S₂O₃ (2M, 10 mL), dried over sodium sulphate, evaporated under reduced pressure to afford a title compound as off white solid (0.1 g, 18.90%), Rf=0.3 (100% n-Hexane); ¹H NMR (300 MHz, Chloroform-d) δ 8.56 (s, 1H), 8.48 (s, 1H); HPLC: 99.35%, retention time=7.08 min (Method E).

### Preparation of 3-fluoro-4-iodo-5-(trifluoromethyl)pyridine

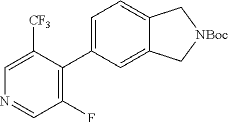 [SMILES: CC(C)(C)OC(=O)N1Cc2ccc(-c3c(F)cncc3C(F)(F)F)cc2C1]

Title compound was prepared from intermediate I-4 using Suzuki coupling procedure F and 3-fluoro-4-iodo-5-(trifluoromethyl)pyridine. The crude compound was purified by column chromatography using 5-15% EtOAc in n-hexanes to afford title compound as off white solid 0.095 g (86.3%, yield) ¹H NMR (300 MHz, Chloroform-d) δ 8.82 (s, 1H), 8.73 (s, 1H), 7.75-7.63 (m, 1H), 7.42-7.29 (m, 1H), 7.23-7.12 (m, 1H), 4.91-4.49 (m, 4H), 1.55 (s, 9H).

### Preparation of 5-(3-fluoro-5-(trifluoromethyl)pyridin-4-yl)isoindoline trifluoroacetic Acid Salt

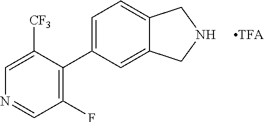 [SMILES: Fc1cncc(C(F)(F)F)c1-c1ccc2c(c1)CNC2 ; "·TFA"]

Title compound was prepared from precursor (0.09 g) using Boc-deprotection procedure A. The crude material taken as such for next step (0.09 g). LC-MS m/z 283.00 [M+H]⁺, retention time=1.25 min (Method A).

### Preparation of 1-(2-(5-(3-fluoro-5-(trifluoromethyl)pyridin-4-yl)isoindolin-2-yl)-2-oxoethyl)-1H-1,2,4-triazole-3-carbonitrile (16)

Compound 16 was prepared from intermediate I-3 using acid amide coupling procedure A. Purification with reversed-phase HPLC (Method D) provided 16 (11 mg, 11.22% yield) as white solid. ¹H NMR (600 MHz, DMSO-d6): δ 9.04 and 9.03 (s, 1H), 8.96 (s, 1H), 8.90-8.88 (m, 1H), 7.55 (t, J=6.9 Hz, 1H), 7.46 and 7.44 (s, 1H), 7.38-7.36 (m, 1H), 5.52 (s, 2H), 5.04 and 5.02 (s, 2H), 4.78 and 4.75 (s, 2H) (NMR shows doubling of protons due to presence of rotamers); LC-MS m/z 416.95 [M+H]⁺, retention time=1.47 min (Method A); HPLC: 99.06%, retention time=3.72 min (Method D).

### Example 17: 1-(2-oxo-2-(5-(2-(trifluoromethoxy)pyridin-3-yl)isoindolin-2-yl)ethyl)-1H-1,2,4-triazole-3-carbonitrile

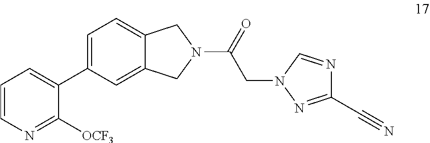 [SMILES: N#Cc1ncn(CC(=O)N2Cc3ccc(-c4cccnc4OC(F)(F)F)cc3C2)n1]

### Preparation of 6-chloro-3-iodo-2-(trifluoromethoxy)pyridine

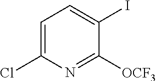 [SMILES: FC(F)(F)Oc1nc(Cl)ccc1I]

To a cooled solution of 2-chloro-6-(trifluoromethoxy)pyridine (0.3 g, 1.51 mmol) in anhydrous THF (2 mL) was added LDA (2M in THF, 0.9 mL, 1.82 mmol) dropwise over 5 min and the resulting solution was stirred for 2 h at −78° C. Iodine (0.5 g, 1.97 mmol) in anhydrous THF (1 mL) was then added dropwise over 5 min at −78° C. and stirred for an hour at same temperature. The reaction mixture was then quenched with saturated aq.NH₄Cl solution (5 mL). Crude was extracted with EtOAc (10 mL×2) and combined organic phases were washed with Na₂S₂O₃ (2M, 10 mL), dried over sodium sulphate, evaporated under reduced pressure to afford a title compound as colourless liquid (0.30 g, 61.07%), Rf=0.6 (05% EtOAc in n-Hexane); ¹H NMR (300 MHz, Chloroform-d) δ 8.09 (d, J=8.0 Hz, 1H), 7.02 (d, J=8.2 Hz, 1H).

### Preparation of tert-butyl 5-(6-chloro-2-(trifluoromethoxy)pyridin-3-yl)isoindoline-2-carboxylate

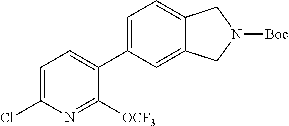 [SMILES: CC(C)(C)OC(=O)N1Cc2ccc(-c3ccc(Cl)nc3OC(F)(F)F)cc2C1]

Title compound was prepared from intermediate I-4 using Suzuki coupling procedure F. The crude compound was purified by column chromatography using 0-20% EtOAc in n-hexanes to afford product as yellow sticky mass 0.19 g (53.60%, yield) ¹H NMR (300 MHz, Chloroform-d) δ 7.74 (dd, J=7.9, 2.6 Hz, 1H), 7.43-7.26 (m, 4H), 4.74 (s, 2H), 4.71 (s, 2H), 1.52 (s, 9H); HPLC: 76.57%, retention time=8.24 min (Method F).

### Preparation of tert-butyl 5-(6-chloro-2-(trifluoromethoxy)pyridin-3-yl)isoindoline-2-carboxylate

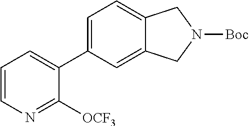 [SMILES: CC(C)(C)OC(=O)N1Cc2ccc(-c3cccnc3OC(F)(F)F)cc2C1]

To a stirred solution of precursor (0.1 g, 0.241 mmol) in methanol (3 mL), 10% Pd/C (0.03 g) and ammonium formate (0.061 g, 0.964 mmol) were added at rt. Reaction mixture was then stirred at 60° C. for 5 hours. After completion of reaction (monitored by TLC), the reaction mixture was cooled to rt, filtered through celite bed. The celite bed was washed with methanol (20 mL), evaporated the solvent in vacuo to dryness. The crude compound was purified by prep TLC method to afford title compound as colorless sticky mass 0.07 g (76.92%, yield) ¹H NMR (300 MHz, Chloroform-d) δ 8.30 (dd, J=4.8, 1.9 Hz, 1H), 7.87-7.70 (m, 1H), 7.46-7.24 (m, 4H), 4.75 (s, 2H), 4.71 (s, 2H), 1.53 (s, 9H).

### Preparation of 5-(2-(trifluoromethoxy)pyridin-3-yl)isoindoline trifluoroacetic Acid Salt

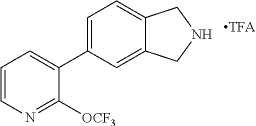 [SMILES: FC(F)(F)Oc1ncccc1-c1ccc2c(c1)CNC2]

Title compound was prepare from precursor 0.07 g) using TFA procedure A. The crude compound was purified by triturating in pentane to afford title compound as light brown sticky mass 0.07 g. LC-MS m/z 280.8 [M+H]⁺, retention time=0.14 min (Method B).

### Preparation of 1-(2-oxo-2-(5-(2-(trifluoromethoxy)pyridin-3-yl)isoindolin-2-yl)ethyl)-1H-1,2,4-triazole-3-carbonitrile(17)

Compound 17 was prepared from intermediate I-3 using acid amide coupling procedure A. Purification with reversed-phase HPLC (Method E) provided 7 (35 mg, 47.94% yield) as white solid. ¹H NMR (400 MHz, Chloroform-d) δ 8.46 (s, 1H), 8.36-8.32 (m, 1H), 7.80 (dd, J=8.0 Hz and 2.0 Hz, 1H), 7.50-7.41 (m, 3H), 7.36-7.31 (m, 1H), 5.17 (s, 2H), 5.03 and 5.02 (s, 2H), 4.92 (s, 2H) (NMR shoes doubling of protons due to presence of rotamers); LC-MS m/z 415.10 [M+H]⁺, retention time=1.51 min (Method A); HPLC: 99.46%, retention time=5.97 min (Method F).

### Example 18: 1-(2-(5-(4-chloro-2-(trifluoromethyl)pyridin-3-yl)isoindolin-2-yl)-2-oxoethyl)-1H-1,2,4-triazole-3-carbonitrile

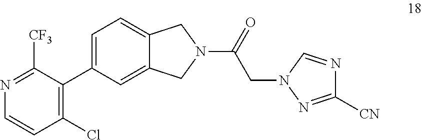 [SMILES: N#Cc1ncn(CC(=O)N2Cc3ccc(-c4c(Cl)ccnc4C(F)(F)F)cc3C2)n1]

### Preparation of 4-chloro-2-(trifluoromethyl)nicotinic Acid

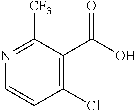 [SMILES: O=C(O)c1c(Cl)ccnc1C(F)(F)F]

To a cooled solution of 2,2,6,6-tetramethylpiperidine (4.43 g, 31.39 mmol) in anhydrous THF (50 mL) was added n-BuLi (2.5M in hexane, 16.74 mL, 41.86 mmol) dropwise over 5 min at −78° C. and the resulting solution was stirred for 30 minutes at −78° C. Then 2-(trifluoromethyl)nicotinic acid (2.0 g, 10.46 mmol) in anhydrous THF (20 mL) was added dropwise over 5 min at −78° C. and the resulting solution was stirred for 20 minutes at −78° C., followed by −50° C. for 1 hour. Hexachloroethane (7.43 g, 31.39 mmol) in anhydrous THF (30 mL) was then added dropwise over 5 min at −78° C. The reaction mixture was allowed to warm to rt and stirred for 1 h at rt. The reaction mixture was then quenched with water, the THF was removed by reduced pressure, to the resulting aqeuous layer was washed with 50% diethyl ether in pentane (50 mL) and adjusted pH 4-5 using 1.0 M solution of HCl and extracted with EtOAc (100 mL×3), combined organic phases were dried over sodium sulphate, evaporated under reduced pressure to afford a title compound as tan solid (1.50 g, 63.57%), Rf=0.1 (5% methanol in Dichloromethane) ¹H NMR (300 MHz, DMSO-d6) δ 8.80 (d, J=5.3 Hz, 1H), 8.06 (d, J=5.3 Hz, 1H); LC-MS m/z 225.95 [M+H]⁺, retention time=0.32 min (Method A).

### Preparation of 4-chloro-2-(trifluoromethyl)nicotinoyl chloride (Step-1)

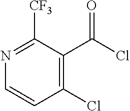 [SMILES: O=C(Cl)c1c(Cl)ccnc1C(F)(F)F]

A mixture of 4-chloro-2-(trifluoromethyl)nicotinic acid (1.5 g, 6.65 mmol) in thionyl chloride (10.0 mL) was stirred at 100° C. for 5 h. The reaction mixture was concentrated in vacuo to afford the title compound (1.60 g, Crude) Rf=0.9 (5% methanol in Dichloromethane).

### Preparation of 4-chloro-2-(trifluoromethyl)nicotinamide

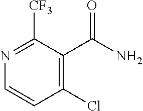 [SMILES: NC(=O)c1c(Cl)ccnc1C(F)(F)F]

To a solution of 4-chloro-2-(trifluoromethyl)nicotinoyl chloride (1.6 g, 6.55 mmol) in toluene (5.0 mL) was added aqueous ammonia (80 mL) drop wise at 0° C. Reaction mixture was stirred at room temperature for 3 h. Reaction mixture diluted with water and extracted with ethylacetate (50 mL×2). The combined organic layer was dried over sodium sulphate and concentrated in vacuo. The crude compound was purified by Column chromatography using 80-100% EtOAc in n-Hexane to afford title compound as tan solid (1.10 g, 74.82%) Rf=0.4 (50% EtOAc in n-Hexane); ¹H NMR (300 MHz, DMSO-d6) δ 8.74 (d, J=5.2 Hz, 1H), 8.24 (bs, 2H), 8.00 (d, J=5.3 Hz, 1H); LC-MS m/z 224.95 [M+H]⁺, retention time=0.63 min (Method A); HPLC: 96.15%, retention time=4.85 min (Method E).

### Preparation of 4-chloro-2-(trifluoromethyl)pyridin-3-amine

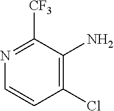 [SMILES: Nc1c(Cl)ccnc1C(F)(F)F]

To a solution of 4-chloro-2-(trifluoromethyl)nicotinamide (1.0 g, 4.5 mmol) in acetonitrile (10 mL) and water (10 mL) was added Bis (Trifluoro acetoxy iodo benzene) (2.10 g, 4.89 mmol) at room temperature. The reaction mixture was stirred at room temperature for 24 h. Reaction mixture diluted with saturated sodium bicarbonate solution and extracted with ethylacetate (100 mL×3). The combined organic layer was washed with brine solution, dried over sodium sulphate and concentrated in vacuo. The crude compound was purified by Column chromatography using 80-100% EtOAc in n-Hexane to afford title compound as tan solid (0.55 g, 62.85%) Rf=0.8 (30% EtOAc in n-Hexane); ¹H NMR (300 MHz, Chloroform-d) δ 7.97 (d, J=4.9 Hz, 1H), 7.39 (d, J=4.8 Hz, 1H), 4.68 (s, 2H). LC-MS m/z 237.95 [M+H+ACN]⁺, retention time=1.42 min (Method A); HPLC: 99.60%, retention time=5.07 min (Method F).

### Preparation of 3-bromo-4-chloro-2-(trifluoromethyl)pyridine

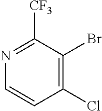 [SMILES: FC(F)(F)c1nccc(Cl)c1Br]

To a solution of 4-chloro-2-(trifluoromethyl)pyridin-3-amine (0.50 g, 2.54 mmol) in acetonitrile (10 mL) was added Copper (II) bromide (0.85 g, 3.81 mmol) lot wise at 0° C., then t-Butyl nitrite (0.52 g, 5.08 mmol) was added drop wise at 0° C. and stirred at room temperature for 5 h. The reaction mixture was concentrated in vacuo, then the residue was dissolved in ethyl acetate and then washed with water, followed by brine solution, dried over sodium sulphate and concentrated in vacuo. The crude compound was purified by Column chromatography using 0-10% EtOAc in n-Hexane to afford title compound as pale yellow liquid, 0.45 g (67.71%). Rf=0.90 (30% EtOAc in n-Hexane); ¹H NMR (300 MHz, Chloroform-d) δ 8.51 (d, J=5.2 Hz, 1H), 7.62 (d, J=5.1 Hz, 1H); HPLC: 95.27%, retention time=7.46 min (Method E).

### Preparation of tert-butyl 5-(4-chloro-2-(trifluoromethyl)pyridin-3-yl)isoindoline-2-carboxylate

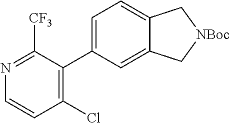 [SMILES: CC(C)(C)OC(=O)N1Cc2ccc(-c3c(Cl)ccnc3C(F)(F)F)cc2C1]

Title compound was prepared from intermediate I-4 using Suzuki coupling procedure F and 3-bromo-4-chloro-2-(trifluoromethyl)pyridine. The crude compound was purified by column chromatography using 0-30% EtOAc in n-hexanes to afford title compound as colourless liquid 0.3 g (49.22%, yield) ¹H NMR (300 MHz, Chloroform-d) δ 8.62 (d, J=5.3 Hz, 1H), 7.65 (d, J=5.3 Hz, 1H), 7.36 (dd, J=15.0, 7.9 Hz, 1H), 7.20-7.01 (m, 2H), 4.86-4.67 (m, 4H), 1.53 (s, 9H); HPLC: 97.59%, retention time=7.56 min (Method F).

### Preparation of 5-(4-chloro-2-(trifluoromethyl)pyridin-3-yl)isoindoline trifluoroacetic Acid Salt

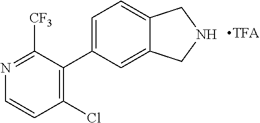 [SMILES: FC(F)(F)c1nccc(Cl)c1-c1ccc2c(c1)CNC2]

Title compound was prepared from precursor (0.3 g) using Boc-deprotection procedure A. The crude material taken as such for next step (0.3 g). LC-MS m/z 298.95 [M+H]⁺, retention time=1.27 min (Method A).

### Preparation of 1-(2-(5-(4-chloro-2-(trifluoromethyl)pyridin-3-yl)isoindolin-2-yl)-2-oxoethyl)-1H-1,2,4-triazole-3-carbonitrile (18)

Compound 18 was prepared from intermediate I-3 using acid amide coupling procedure A. The crude compound was purified by column chromatography using 0-80% EtOAc in n-hexanes, further purified by dissolving in chloroform and adding pentane till precipitation to afford title compound as white solid 0.095 g (29.20%, yield). ¹H NMR (300 MHz, Chloroform-d) δ 8.64 (d, J=6.3 Hz, 1H), 8.46 (s, 1H), 7.70-7.66 (m, 1H), 7.44 (t, J=7.4 Hz, 1H), 7.26-7.18 (m, 2H), 5.17 (s, 2H), 5.05 and 5.04 (s, 2H), 4.95 and 4.93 (s, 2H) (NMR shows doubling of protons due to presence of rotamers); LC-MS m/z 432.90 [M+H]⁺, retention time=1.49 min (Method A); HPLC: 99.62%, retention time=5.79 min (Method F).

### Example 19: 1-(2-oxo-2-(5-(2-(2,2,2-trifluoroethyl)pyridin-3-yl)isoindolin-2-yl)ethyl)-1H-1,2,4-triazole-3-carbonitrile

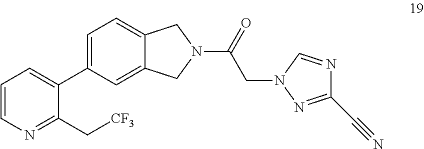 [SMILES: N#Cc1ncn(CC(=O)N2Cc3ccc(-c4cccnc4CC(F)(F)F)cc3C2)n1]

### Preparation of 1-(3-bromopyridin-1-yl)-2,2,2-trifluo ethan-1-ol

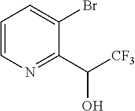 [SMILES: OC(c1ncccc1Br)C(F)(F)F]

To a solution of 3-bromopicolinaldehyde (1 g, 5.43 mmol) in THF (15 mL) was added Cesium fluoride (1.23 g, 8.15 mmol) at 0° C. followed by dropwise addition of TMSCF₃ (1.08 g, 6.52 mmol). Reaction mixture was stirred at 0° C. for 1 h. Reaction mixture diluted with water and extracted with EtOAc (100 mL×2). The combined organic layer was dried over sodium sulphate and concentrated in vacuo. Crude compound was purified by column chromatography using 0-20% EtOAc in n-hexanes to afford title compound 0.5 g (36.2%), Rf=0.35 (10% EtOAc in n-Hexane). ¹H NMR (300 MHz, Chloroform-d) δ 8.60 (dd, J=4.7, 1.4 Hz, 1H), 7.97 (dd, J=8.1, 1.4 Hz, 1H), 7.29 (dd, J=8.1, 4.7 Hz, 1H), 5.46 (dq, J=9.7, 5.9 Hz, 1H), 4.82 (d, J=9.9 Hz, 1H).

### Preparation of 1-(3-bromopyridin-2-yl)-2,2,2-trifluoroethyl methanesulfonate

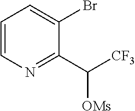 [SMILES: CS(=O)(=O)OC(c1ncccc1Br)C(F)(F)F]

To a solution of precursor (300 mg, 1.18 mmol) in DCM (10 mL) was added DIPEA (238 mg, 2.36 mmol) in portions at 0° C. followed by addition of methanesulfonylchloride (201 mg, 1.77 mmol). Reaction mixture was stirred at 0° C. for 2 h. Reaction mixture diluted with water and extracted with DCM (25 mL×2). The combined organic layer was dried over sodium sulphate and concentrated at reduced pressure to get title compound (350 mg), which was used for next step without further purification. Rf=0.3 (20% EtOAc in n-Hexane); ¹H NMR (300 MHz, Chloroform-d) δ 8.71 (dd, J=4.6, 1.5 Hz, 1H), 7.99 (dd, J=8.1, 1.5 Hz, 1H), 7.31 (dd, J=8.3, 4.6 Hz, 1H), 6.55 (q, J=5.9 Hz, 1H), 3.67 (s, 3H); LC-MS m/z 335.90 [M+H]⁺, retention time=1.47 min (Method A).

### Preparation of tert-butyl 5-(2-(2,2,2-trifluoroethyl)pyridin-3-yl)isoindoline-2-carboxylate

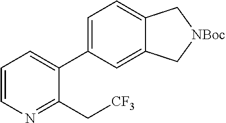 [SMILES: CC(C)(C)OC(=O)N1Cc2ccc(-c3cccnc3CC(F)(F)F)cc2C1]

Title compound was prepared from precursor using Suzuki coupling method J and boronate ester I-4. Purification with silica gel column chromatography using 0-20% EtOAc in n-hexanes provided product (80 mg, 28.1% yield). ¹H NMR (300 MHz, Chloroform-d) δ 8.66 (d, J=4.4 Hz, 1H), 7.58 (d, J=8.0 Hz, 1H), 7.33 (dd, J=8.2, 5.5 Hz, 2H), 7.22-7.09 (m, 2H), 4.75 (s, 2H), 4.72 (s, 2H), 3.58 (q, J=10.2 Hz, 2H), 1.56 (s, 9H).

### Preparation of 5-(2-(2,2,2-trifluoroethyl)pyridin-3-yl)isoindoline

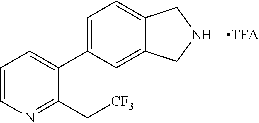 [SMILES: FC(F)(F)Cc1ncccc1-c1ccc2c(c1)CNC2 ; "·TFA"]

Title compound was prepared from using Boc-deprotection procedure A. The crude material (80 mg) taken for the next step without purification. LC-MS m/z 279.10 [M+H]⁺, retention time=1.23 min (Method A).

### Preparation of 1-(2-oxo-2-(5-(2-(2,2,2-trifluoroethyl)pyridin-3-yl)isoindolin-2-yl)ethyl)-1H-1,2,4-triazole-3-carbonitrile (19)

Compound 19 was prepared using acid-amine coupling procedure A and acid intermediate I-3. Purification with Prep-TLC plate to get 19 (18 mg, 22.5% yield) as off white solid. ¹H NMR (400 MHz, Chloroform-d): δ 8.69 (bs, 1H), 8.46 (d, J=2.4 Hz, 1H), 7.61 (dd, J=8.0, 1.6 Hz, 1H), 7.43 (t, J=8.0 Hz, 1H), 7.40-7.35 (m, 1H), 7.30-7.26 (m, 2H), 5.18 and 5.17 (s, 2H), 5.04 and 5.03 (s, 2H), 4.94 and 4.92 (s, 2H), 3.60 (q, J=10.4 Hz, 2H) (NMR shows doubling of protons due to presence of rotamers); LC-MS m/z 412.7 [M+H]⁺, retention time=1.21 min (Method D); HPLC: 95.08%, retention time=3.58 min (Method D).

### Example 20: 1-(2-(5-(4-fluoro-2-(trifluoromethyl)pyridin-3-yl)isoindolin-2-yl)-2-oxoethyl)-1H-1,2,4-triazole-3-carbonitrile

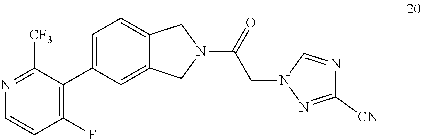 [SMILES: N#Cc1ncn(CC(=O)N2Cc3ccc(-c4c(F)ccnc4C(F)(F)F)cc3C2)n1]

### Preparation of 3,5-dibromo-2-(trifluoromethyl)pyridin-4-amine

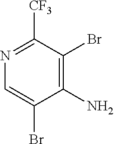 [SMILES: Nc1c(Br)cnc(C(F)(F)F)c1Br]

A stirred solution of 2-(trifluoromethyl)pyridin-4-amine (1.5 g, 9.25 mmol) in acetonitrile (30 mL) was added NBS (4.94 g, 27.75 mmol) and stirred at RT for 30 h. Reaction mixture was concentrated to dryness, stirred with CCl₄, filtered and filtrate was concentrated to obtain crude. Product was purified by combi flash using 10%-20% EtOAc in Hexane to afford title compound (2.5 g, 84.45%), Rf=0.6 (50% EtOAc in n-Hexane); ¹H NMR (300 MHz, Chloroform-d) δ 8.37 (s, 1H), 5.44 (s, 2H); LC-MS m/z 320.70 [M+H]⁺, retention time=1.51 min (Method A); HPLC: 94.58%, retention time=6.04 min (Method E).

### Preparation of 3-bromo-2-(trifluoromethyl)pyridin-4-amine

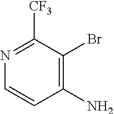 [SMILES: Nc1ccnc(C(F)(F)F)c1Br]

A stirred solution of precursor (2.5 g, 7.81 mmol) in EtOH (40 mL) was added 10% Pd/C (50% wet) (0.25 g, 10% w/w) and stirred at RT for 3 h. Reaction mixture was filtered through celite and filtrate was concentrated. Product was recrystallized from EtOH and dried to afford title compound (1.5 g, 79.78%), Rf=0.3 (50% EtOAc in n-Hexane); ¹H NMR (300 MHz, DMSO-d6) δ 8.05 (d, J=5.5 Hz, 1H), 6.89 (d, J=5.6 Hz, 1H), 5.30 (s, 2H); LC-MS m/z 240.85 [M+H]⁺, retention time=1.42 min (Method A); HPLC: 76.03%, retention time=6.13 min (Method E).

### Preparation of tert-butyl 5-(4-amino-2-(trifluoromethyl)pyridin-3-yl)isoindoline-2-carboxylate

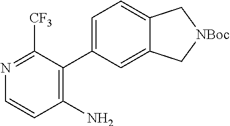 [SMILES: CC(C)(C)OC(=O)N1Cc2ccc(-c3c(N)ccnc3C(F)(F)F)cc2C1]

Title was prepared from intermediate I-4 using Suzuki coupling procedure F and precursor (500 mg, 2.07 mmol). The crude compound was purified by column chromatography using 30-50% EtOAc in n-hexanes to afford title compound (320 mg, 40.66%). ¹H NMR (300 MHz, Chloroform-d) δ 8.29 (d, J=5.5 Hz, 1H), 7.37 (dd, J=14.7, 7.7 Hz, 1H), 7.20-7.07 (m, 2H), 6.75 (d, J=5.6 Hz, 1H), 4.83-4.66 (m, 4H), 1.52 (s, 9H); LC-MS m/z 380.00 [M+H]⁺, retention time=1.49 min (Method A); HPLC: 96.33%, retention time=3.75 min (Method D).

### Preparation of tert-butyl 5-(4-iodo-2-(trifluoromethyl)pyridin-3-yl)isoindoline-2-carboxylate

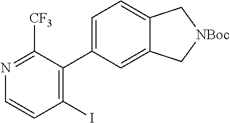 [SMILES: CC(C)(C)OC(=O)N1Cc2ccc(-c3c(I)ccnc3C(F)(F)F)cc2C1]

A stirred solution of Iodine (535 mg, 2.11 mmol) and isoamyl nitrite (394 mg, 3.36 mmol) in ACN (3 mL) was added precursor amine (320 mg, 0.84 mmol) in ACN (1 mL) at 0° C. and stirred at RT for 1 h. Reaction mixture was quenched with saturated sodium thiosulfate solution and extracted with EtOAc (20 ml×2). Combined organic layer was washed with water, brine and dried over anhydrous Na₂SO₄. Organic layer was concentrated to obtain crude product. Product was purified by combi flash column chromatography using 20% EtOAc in n-hexanes, resulting product was stirred and filtered to afford title compound 0.120 g (29.05%), Rf=0.6 (20% EtOAc in n-Hexane); ¹H NMR (300 MHz, Chloroform-d) δ 8.28 (d, J=5.1 Hz, 1H), 8.11 (d, J=5.1 Hz, 1H), 7.35 (dd, J=14.5, 7.7 Hz, 1H), 7.14-6.96 (m, 2H), 4.86-4.65 (m, 4H), 1.52 (s, 9H); HPLC: 95.28%, retention time=6.49 min (Method C).

### Preparation of tert-butyl 5-(4-fluoro-2-(trifluoromethyl)pyridin-3-yl)isoindoline-2-carboxylate

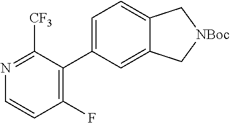 [SMILES: CC(C)(C)OC(=O)N1Cc2ccc(-c3c(F)ccnc3C(F)(F)F)cc2C1]

A stirred solution of iodo-precursor (120 mg, 0.42 mmol) in DMSO (4 mL) was added cesium fluoride (651 mg, 4.28 mmol) and stirred at 100° C. for 10 h. Reaction mixture was quenched with ice cold water and extracted with EtOAc (20 ml×2). Combined organic layer was washed with water, brine and dried over anhydrous Na₂SO₄. Organic layer was concentrated to obtain crude product. Product was purified by combi flash column chromatography using 20% EtOAc in n-hexanes to afford title compound 0.050 g (64.10%), Rf=0.5 (20% EtOAc in n-Hexane); ¹H NMR (300 MHz, Chloroform-d) δ 8.70 (dd, J=7.3, 5.4 Hz, 1H), 7.47-7.29 (m, 2H), 7.21-7.25 (m, 2H), 4.92-4.60 (m, 4H), 1.52 (s, 9H); HPLC: 91.25%, retention time=7.23 min (Method F).

### Preparation of 5-(4-fluoro-2-(trifluoromethyl)pyridin-3-yl)isoindoline trifluoroacetic Acid Salt

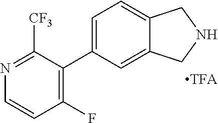 [SMILES: Fc1ccnc(C(F)(F)F)c1-c1ccc2c(c1)CNC2]

Title compound was prepared from precursor (50 mg, 0.13 mmol) using Boc-deprotection procedure A. The crude material was taken to next step as such (40 mg). LC-MS m/z 283.00 [M+H]⁺, retention time=1.26 min (Method A).

### Preparation of 1-(2-(5-(4-fluoro-2-(trifluoromethyl)pyridin-3-yl)isoindolin-2-yl)-2-oxoethyl)-1H-1,2,4-triazole-3-carbonitrile (20)

Compound 20 was prepared from intermediate I-3 using acid amide coupling procedure A and precursor amine (40 mg, 0.20 mmol). The crude compound was purified by column chromatography using 70-90% EtOAc in Hexane to afford title compound as off white solid (19 mg, 45.23%). ¹H NMR (400 MHz, Chloroform-d) δ 8.76-8.71 (m, 1H), 8.46 (s, 1H), 7.45 (t, J=8.4 Hz, 1H), 7.39-7.28 (m, 3H), 5.17 and 5.16 (s, 2H), 5.04 (s, 2H), 4.94 and 4.93 (s, 2H). LC-MS m/z 414.85 [M−H]⁺, retention time=1.47 min (Method A); HPLC: 98.01%, retention time=5.54 min (Method F).

### Example 21: 1-(2-(5-(2-chloro-4-fluoropyridin-3-yl)isoindolin-2-yl)-2-oxoethyl)-1H-1,2,4-triazole-3-carbonitrile

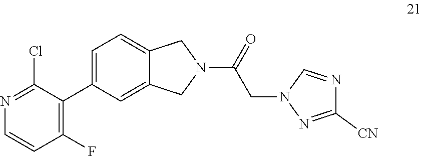 [SMILES: N#Cc1ncn(CC(=O)N2Cc3ccc(-c4c(F)ccnc4Cl)cc3C2)n1]

### Preparation of tert-butyl 5-(2-chloro-4-fluoropyridin-3-yl)isoindoline-2-carboxylate

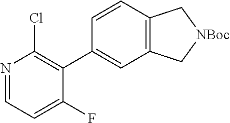 [SMILES: CC(C)(C)OC(=O)N1Cc2ccc(-c3c(F)ccnc3Cl)cc2C1]

Title compound was prepared from 2-chloro-4-fluoro-3-iodopyridine using Suzuki coupling procedure F and boronate ester I-4. Purification with silica gel column chromatography using 0-30% EtOAc in n-hexanes provided product (120 mg, 59.1% yield). ¹H NMR (300 MHz, Chloroform-d) δ 8.38 (dd, J=7.8, 5.5 Hz, 1H), 7.42-7.34 (m, 1H), 7.32-7.19 (m, 2H), 7.12 (dd, J=8.0, 5.6 Hz, 1H), 4.76 (s, 2H), 4.73 (s, 2H), 1.52 (s, 9H).

### Preparation of 5-(2-chloro-4-fluoropyridin-3-yl)isoindoline

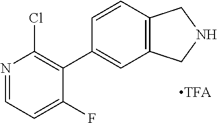 [SMILES: Fc1ccnc(Cl)c1-c1ccc2c(c1)CNC2]

Title amine was prepared using Boc-deprotection procedure A. The crude material (100 mg) taken for the next step without purification. LC-MS m/z 248.90 [M+H]⁺, retention time=1.21 min (Method A).

### Preparation of 1-(2-(5-(2-chloro-4-fluoropyridin-3-yl)isoindolin-2-yl)-2-oxoethyl)-1H-1,2,4-triazole-3-carbonitrile (21)

Compound 21 was prepared using acid-amine coupling procedure A and acid intermediate I-3. Purification with silica gel column chromatography using 30-100% EtOAc in n-hexanes provided 21 (12 mg, 10.9% yield) as white solid. ¹H NMR (400 MHz, DMSO-d6): δ 8.90 and 8.89 (s, 1H), 8.50 (t, J=6.0 Hz, 1H), 7.60-7.52 (m, 2H), 7.47 (d, J=9.2 Hz, 1H), 7.38 (d, J=8.0 Hz, 1H), 5.52 (s, 2H), 5.02 (s, 2H), 4.77 and 4.75 (s, 2H); (NMR shows doubling of protons due to presence of rotamers); LC-MS m/z 382.90 [M+H]⁺, retention time=1.45 min (Method A); HPLC: 98.73%, retention time=5.25 min (Method F).

### Example 22: 1-(2-(5-(2-fluoro-4-(trifluoromethyl)pyridin-3-yl)isoindolin-2-yl)-2-oxoethyl)-1H-1,2,4-triazole-3-carbonitrile

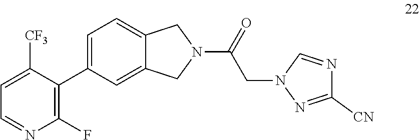 [SMILES: N#Cc1ncn(CC(=O)N2Cc3ccc(-c4c(C(F)(F)F)ccnc4F)cc3C2)n1]

### Preparation of 2-fluoro-3-iodo-4-(trifluoromethyl)pyridine

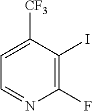 [SMILES: Fc1nccc(C(F)(F)F)c1I]

To a cooled solution of 2-fluoro-4-(trifluoromethyl)pyridine (200 mg, 1.21 mmol) in anhydrous THF (10 mL) was added LDA (2M in THF, 0.9 mL, 1.81 mmol) dropwise over 5 min and the resulting solution was stirred for 2 h at −78° C. Iodine (0.5 g, 1.81 mmol) in anhydrous THF (2 mL) was then added dropwise over 5 min at −78° C., causing the colour of the solution to change to red-brown. After 30 min the mixture is allowed to warm and stirred for 1 h at rt. The reaction mixture was cooled again to 0° C. and then quenched with saturated aq.NH₄Cl solution (5 mL). Crude was extracted with EtOAc (10 mL×2) and combined organic phases were washed with Na₂S₂O₃ (2M, 10 mL), dried over sodium sulphate, evaporated under reduced pressure to afford a title compound as yellow oil (250 mg, 71.0%), Rf=0.5 (10% EtOAc in n-Hexane); ¹H NMR (400 MHz, Chloroform-d) δ 8.31 (d, J=5.1 Hz, 1H), 7.43 (d, J=5.0 Hz, 1H).

### Preparation of tert-butyl 5-(2-fluoro-4-(trifluoromethyl)pyridin-3-yl)isoindoline-2-carboxylate

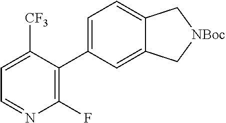 [SMILES: CC(C)(C)OC(=O)N1Cc2ccc(-c3c(C(F)(F)F)ccnc3F)cc2C1]

Title compound was prepared using Suzuki coupling procedure F and boronate ester I-4. Purification with silica gel column chromatography using 0-20% EtOAc in n-hexanes provided product (130 mg, 66% yield). LC-MS m/z 283.2 [M+H−Boc]⁺, retention time=1.85 min (Method D).

### Preparation of 5-(2-fluoro-4-(trifluoromethyl)pyridin-3-yl)isoindoline

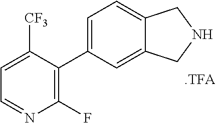 [SMILES: Fc1nccc(C(F)(F)F)c1-c1ccc2c(c1)CNC2]

Title amine was prepared using Boc-deprotection procedure A. The crude material (130 mg) taken for the next step without purification, LC-MS m/z 282.90 [M+H]⁺, retention time=1.27 min (Method A).

### Preparation of 1-(2-(5-(2-fluoro-4-(trifluoromethyl)pyridin-3-yl)isoindolin-2-yl)-2-oxoethyl)-1H-1,2,4-triazole-3-carbonitrile (22)

Compound 22 was prepared using acid-amine coupling procedure A and acid intermediate I-3. Purification with silica gel column chromatography using 20-100% EtOAc in n-hexanes provided 22 (40 mg, 28.1% yield) as off white solid. ¹H NMR (600 MHz, DMSO-d6): δ 8.90 and 8.89 (s, 1H), 8.58 (bs, 1H), 7.88 (s, 1H), 7.56-7.50 (m, 1H), 7.46 and 7.44 (s, 1H), 7.38-7.35 (m, 1H), 5.52 (s, 2H), 5.04 and 5.02 (s, 2H), 4.74 and 4.78 (s, 2H) (NMR shows doubling of protons due to presence of rotamers); LC-MS m/z 416.90 [M+H]⁺, retention time=1.49 min (Method A); HPLC: 99.61%, retention time=4.02 min (Method C).

### Example 23: 1-(2-(5-(3-chloro-5-fluoropyridin-4-yl)isoindolin-2-yl)-2-oxoethyl)-1H-1,2,4-triazole-3-carbonitrile

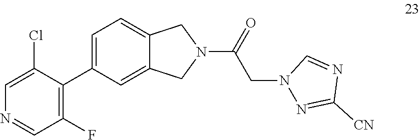 [SMILES: N#Cc1ncn(CC(=O)N2Cc3ccc(-c4c(F)cncc4Cl)cc3C2)n1]

### Preparation of tert-butyl 5-(3-chloro-5-fluoropyridin-4-yl)isoindoline-2-carboxylate

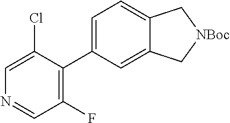 [SMILES: CC(C)(C)OC(=O)N1Cc2ccc(-c3c(F)cncc3Cl)cc2C1]

Title compound was prepared from intermediate I-4 using Suzuki coupling procedure F and 4-bromo-3-chloro-5-fluoropyridine. The crude compound was purified by column chromatography using 5-10% EtOAc in n-hexanes to afford title compound as pale yellow solid 0.09 g (49.52%, yield) ¹H NMR (300 MHz, Chloroform-d) δ 8.55 (s, 1H), 8.47 (s, 1H), 7.46-7.27 (m, 3H), 4.75 (d, J=10.5 Hz, 4H), 1.52 (s, 9H).

### Preparation of 5-(3-chloro-5-fluoropyridin-4-yl)isoindoline trifluoroacetic Acid Salt

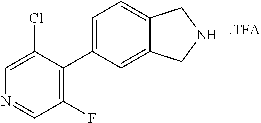 [SMILES: Fc1cncc(Cl)c1-c1ccc2c(c1)CNC2]

Title compound was prepared from precursor (0.09 g) using Boc-deprotection procedure A. The crude material taken as such for next step (0.08 g). LC-MS m/z 248.90 [M+H]⁺, retention time=0.24 min (Method A).

### Preparation of 1-(2-(5-(3-chloro-5-fluoropyridin-4-yl)isoindolin-2-yl)-2-oxoethyl)-1H-1,2,4-triazole-3-carbonitrile (23)

Compound 23 was prepared from intermediate I-3 using acid amide coupling procedure A. The crude compound was purified by column chromatography using 40-70% EtOAc in n-hexanes to afford title compound as brown solid 0.04 g (45.02%, yield). ¹H NMR (400 MHz, DMSO-d6): δ 8.90 and 8.89 (s, 1H), 8.72 and 8.71 (s, 1H), 8.70 and 8.69 (s, 1H), 7.57 (dd, J=7.8 Hz and 4.2 Hz, 1H), 7.50 (d, J=8.4 Hz, 1H), 7.42 (d, J=7.6 Hz, 1H), 5.52 (s, 2H), 5.03 (s, 2H), 4.77 and 4.76 (s, 2H) (NMR shows doubling of protons due to presence of rotamers); LC-MS m/z 382.90 [M+H]⁺, retention time=1.46 min (Method A); HPLC: 98.83%, retention time=6.95 min (Method B).

### Example 24: 1-(2-(5-(4-chloro-2-fluoropyridin-3-yl)isoindolin-2-yl)-2-oxoethyl)-1H-1,2,4-triazole-3-carbonitrile

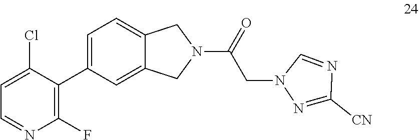 [SMILES: N#Cc1ncn(CC(=O)N2Cc3ccc(-c4c(Cl)ccnc4F)cc3C2)n1]

### Preparation of 4-chloro-2-fluoropyridine

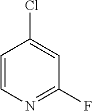

To a solution of compound 4-chloropyridin-2-amine (500 mg, 3.90 mmol) in HF-pyridine (2.5 mL) was added sodium nitrite (296 mg, 4.29 mmol) in portions at 0° C. Reaction mixture was stirred at 10° C. for 1 h. Reaction mixture diluted with water and extracted with DCM (25 mL×2). The combined organic layer was dried over sodium sulphate and concentrated at reduced pressure to get crude title compound as brown liquid (350 mg, 68.46%) Rf=0.7 (100% n-Hexane); ¹H NMR (300 MHz, DMSO-d6) δ 8.27 (dd, J=5.3, 0.8 Hz, 1H), 7.59-7.42 (m, 2H); LC-MS m/z 132.05 [M+H]⁺, retention time=1.40 min (Method A); HPLC: 90.08%, retention time=5.11 min (Method F).

### Preparation of 4-chloro-2-fluoro-3-iodopyridine

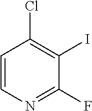

To a cooled solution of 4-chloro-2-fluoropyridine (0.35 g, 2.67 mmol) in anhydrous THF (5 mL) was added LDA (2M in THF, 1.46 mL, 2.93 mmol) dropwise over 5 min and the resulting solution was stirred for 1 h at −78° C. Iodine (0.74 g, 2.93 mmol) in anhydrous THF (2 mL) was then added dropwise over 5 min at −78° C. and stirred for an hour at same temperature. The reaction mixture was then quenched with saturated aq.NH₄Cl solution (5 mL). Crude was extracted with EtOAc (10 mL×2) and combined organic phases were washed with Na₂S₂O₃ (2M, 10 mL), dried over sodium sulphate, evaporated under reduced pressure to afford a title compound as brown solid (0.35 g, 51.20%), Rf=0.6 (100% n-Hexane). ¹H NMR (300 MHz, DMSO-d6) δ 8.18 (d, J=5.3 Hz, 1H), 7.61 (d, J=5.3 Hz, 1H). HPLC: 88.85%, retention time=6.26 min (Method F).

### Preparation of tert-butyl 5-(4-chloro-2-fluoropyridin-3-yl)isoindoline-2-carboxylate

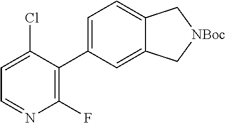 [SMILES: CC(C)(C)OC(=O)N1Cc2ccc(-c3c(Cl)ccnc3F)cc2C1]

Title compound was prepared from intermediate I-4 using Suzuki coupling procedure F and 4-chloro-2-fluoro-3-iodopyridine. The crude compound was purified by column chromatography using 10-20% EtOAc in n-hexanes to afford title compound as pale yellow solid 0.095 g (93.77%, yield) ¹H NMR (400 MHz, Chloroform-d) δ 8.10 (d, J=5.5 Hz, 1H), 7.38 (d, J=7.9 Hz, 1H), 7.35-7.30 (m, 1H), 7.27-7.20 (m, 2H), 4.74 (s, 2H), 4.70 (s, 2H), 1.51 (s, 9H); HPLC: 89.34%, retention time=5.94 min (Method C).

### Preparation of 5-(4-chloro-2-fluoropyridin-3-yl)isoindoline trifluoroacetic Acid Salt

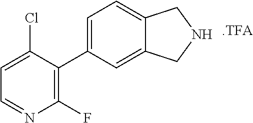 [SMILES: Fc1nccc(Cl)c1-c1ccc2c(c1)CNC2]

Title compound was prepared from precursor (0.095 g) using Boc-deprotection procedure A. The crude material taken as such for next step (0.095 g). LC-MS m/z 246.05 [M−H]⁺, retention time=1.24 min (Method A).

### Preparation of 1-(2-(5-(4-chloro-2-fluoropyridin-3-yl)isoindolin-2-yl)-2-oxoethyl)-1H-1,2,4-triazole-3-carbonitrile (24)

Compound 24 was prepared from intermediate I-3 using acid amide coupling procedure A. The crude compound was purified by column chromatography using 40-70% EtOAc in n-hexanes to afford title compound as brown solid 0.025 g (25.00%, yield). ¹H NMR (400 MHz, DMSO-d6): δ 8.91 and 8.90 (s, 1H), 8.27 (d, J=5.4 Hz, 1H), 7.70 (d, J=5.2 Hz, 1H), 7.58-7.52 (m, 1H), 7.48 (d, J=8.4 Hz, 1H), 7.40 (d, J=8.4 Hz, 1H), 5.53 and 5.52 (s, 2H), 5.03 and 5.02 (s, 2H), 4.77 and 4.75 (s, 2H) (NMR shows doubling of protons due to presence of rotamers); LC-MS m/z 382.95 [M+H]⁺, retention time=1.47 min (Method A); HPLC: 95.84%, retention time=3.90 min (Method C).

### Example 25: 1-(2-(5-(1-methyl-3-(trifluoromethyl)-1H-pyrazol-4-yl)isoindolin-2-yl)-2-oxoethyl)-1H-1,2,4-triazole-3-carbonitrile

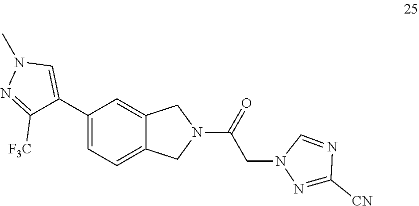 [SMILES: Cn1cc(-c2ccc3c(c2)CN(C(=O)Cn2cnc(C#N)n2)C3)c(C(F)(F)F)n1]

### Preparation of tert-butyl 5-(1-methyl-3-(trifluoromethyl)-1H-pyrazol-4-yl)isoindoline-2-carboxylate

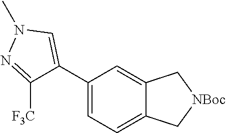 [SMILES: Cn1cc(-c2ccc3c(c2)CN(C(=O)OC(C)(C)C)C3)c(C(F)(F)F)n1]

Title compound was prepared from tert-butyl 5-bromoisoindoline-2-carboxylate using Suzuki coupling procedure I and (1-methyl-3-(trifluoromethyl)-1H-pyrazol-4-yl)boronic acid. Purification with silica gel column chromatography using 0-20% EtOAc in n-hexanes provided 3 (120 mg, 75.0% yield). ¹H NMR (300 MHz, Chloroform-d) δ 7.48 (s, 1H), 7.36-7.20 (m, 3H), 4.71 (s, 2H), 4.68 (s, 2H), 3.99 (s, 3H), 1.52 (s, 12H).

### Preparation of 5-(1-methyl-3-(trifluoromethyl)-1H-pyrazol-4-yl)isoindoline

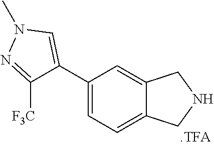 [SMILES: Cn1cc(-c2ccc3c(c2)CNC3)c(C(F)(F)F)n1]

Title compound was prepared using Boc-deprotection procedure A. The crude material (100 mg) taken for the next step without purification. LC-MS m/z 268.00 [M+H]⁺, retention time=1.27 min (Method A)

### Preparation of 1-(2-(5-(1-methyl-3-(trifluoromethyl)-1H-pyrazol-4-yl)isoindolin-2-yl)-2-oxoethyl)-1H-1,2,4-triazole-3-carbonitrile (25)

Compound 25 was prepared using acid-amine coupling procedure A and acid intermediate I-3. Purification with silica gel column chromatography using 20-90% EtOAc in n-hexanes provided 25 (28 mg, 25.6% yield) as off white solid. ¹H NMR (400 MHz, DMSO-d6): δ 8.89 (s, 1H), 8.16 (d, J=6.4 Hz, 1H), 7.48-7.32 (m, 3H), 5.51 (s, 2H), 4.99 (s, 2H), 4.72 (s, 2H), 3.96 (s, 3H);); LC-MS m/z 401.95 [M+H]⁺, retention time=1.47 min (Method A); HPLC: 99.07%, retention time=3.86 min (Method C).

### Example 26: 1-(2-(5-(6-fluoro-2-(trifluoromethyl)pyridin-3-yl)isoindolin-2-yl)-2-oxoethyl)-1H-1,2,4-triazole-3-carbonitrile

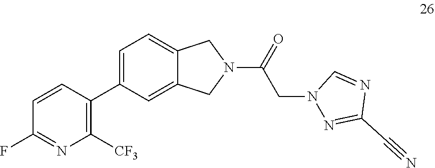 [SMILES: N#Cc1ncn(CC(=O)N2Cc3ccc(-c4ccc(F)nc4C(F)(F)F)cc3C2)n1]

### Preparation of 5-bromo-6-(trifluoromethyl)pyridin-2-amine

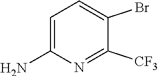 [SMILES: Nc1ccc(Br)c(C(F)(F)F)n1]

To a stirred solution of 6-(trifluoromethyl)pyridin-2-amine (2 g, 12.34 mmol) in methanol (20 mL) was added N-bromosuccinamide (2.19 g, 12.34 mmol) in portions at 0° C. Reaction mixture was then stirred at rt for 10 h. After completion of reaction (monitored by TLC) evaporated the solvent in vacuo to dryness. Crude compound was purified by column chromatography using 0-50% EtOAc in n-hexanes to afford title compound 1.4 g (48.2%), Rf=0.55 (20% EtOAc in n-Hexane). ¹H NMR (300 MHz, Chloroform-d) δ 7.68 (d, J=8.6 Hz, 1H), 6.53 (d, J=8.7 Hz, 1H), 4.71 (s, 2H).

### Preparation of 3-bromo-6-fluoro-2-(trifluoromethyl)pyridine

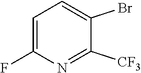 [SMILES: Fc1ccc(Br)c(C(F)(F)F)n1]

To a solution of amine precursor (300 mg, 1.25 mmol) in HF-pyridine (3 mL) was added sodium nitrite (103 mg, 1.50 mmol) in portions at 0° C. Reaction mixture was stirred at 0° C. for 1 h. Reaction mixture diluted with water and extracted with DCM (25 mL×2). The combined organic layer was dried over sodium sulphate and concentrated at reduced pressure (DCM was not evaporated completely due to low boiling point of compound) to get crude title compound (500 mg), which was used for next step without further purification. Rf=0.4 (10% EtOAc in n-Hexane); ¹H NMR (400 MHz, Chloroform-d) δ 8.29-7.97 (m, 1H), 7.07 (ddd, J=8.6, 3.7, 0.6 Hz, 1H).

### Preparation of tert-butyl 5-(6-fluoro-2-(trifluoromethyl)pyridin-3-yl)isoindoline-2-carboxylate

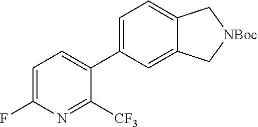 [SMILES: CC(C)(C)OC(=O)N1Cc2ccc(-c3ccc(F)nc3C(F)(F)F)cc2C1]

Title compound was prepared using Suzuki coupling procedure F and boronate ester I-1. Purification with silica gel column chromatography using 0-50% EtOAc in n-hexanes provided product (100 mg, 13.1% yield). ¹H NMR (400 MHz, Chloroform-d) δ 7.84-7.75 (m, 1H), 7.35 □7.26 (m, 1H), 7.21-7.12 (m, 3H), 4.77-4.64 (m, 4H), 1.51 (s, 9H).

### Preparation of 5-(6-fluoro-2-(trifluoromethyl)pyridin-3-yl)isoindoline

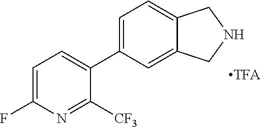 [SMILES: Fc1ccc(-c2ccc3c(c2)CNC3)c(C(F)(F)F)n1]

Title compound was prepare using Boc-deprotection procedure A. The crude material (100 mg) taken for the next step without purification. LC-MS m/z 282.95 [M+H]⁺, retention time=1.29 min (Method A).

### Preparation of 1-(2-(5-(6-fluoro-2-(trifluoromethyl)pyridin-3-yl)isoindolin-2-yl)-2-oxoethyl)-1H-1,2,4-triazole-3-carbonitrile (26)

Compound 26 was prepared using acid-amine coupling procedure A and acid intermediate I-3. Purification with silica gel column chromatography using 20-100% EtOAc in n-hexanes provided 26 (16 mg, 14.6% yield) as brown solid. ¹H NMR (400 MHz, Chloroform-d): δ 8.46 (s, 1H), 7.83 (t, J=8.4 Hz, 1H), 7.42 (t, J=8.4 Hz, 1H), 7.32-7.20 (m, 3H), 5.17 (s, 2H), 5.03 and 5.02 (s, 2H), 4.93 and 4.91 (s, 2H); LC-MS m/z 414.85 [M−H]⁺, retention time=1.51 min (Method A); HPLC: 95.93%, retention time=5.93 min (Method E).

### Example 27: 1-(2-oxo-2-(5-(3-(trifluoromethyl)pyridin-4-yl)isoindolin-2-yl)ethyl)-1H-1,2,4-triazole-3-carbonitrile

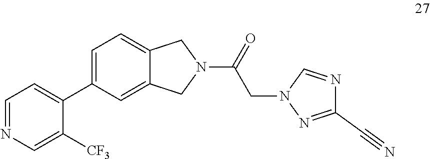 [SMILES: N#Cc1ncn(CC(=O)N2Cc3ccc(-c4ccncc4C(F)(F)F)cc3C2)n1]

Example 27 was prepared from intermediate I-5 using Suzuki coupling procedure B and (3-(trifluoromethyl)pyridin-4-yl)boronic acid. Purification with reversed-phase HPLC (Method H) provided 27 (22% yield) as white solid. ¹H NMR (400 MHz, DMSO-d6) δ 9.06 (s, 1H), 8.97-8.87 (m, 2H), 7.56-7.49 (m, 2H), 7.44 (s, 1H), 7.35 (d, J=7.7 Hz, 1H), 5.58-5.49 (m, 2H), 5.05 (d, J=6.5 Hz, 2H), 4.78 (d, J=10.4 Hz, 2H); LC-MS m/z 399 [M+H]⁺, retention time=0.88 min (Method A).

### Example 28: 1-(2-(5-(5-fluoro-2-(trifluoromethyl)phenyl)isoindolin-2-yl)-2-oxoethyl)-1H-1,2,4-triazole-3-carbonitrile

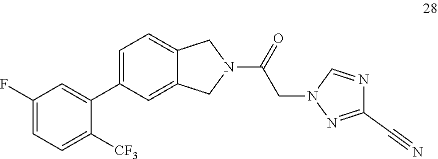 [SMILES: N#Cc1ncn(CC(=O)N2Cc3ccc(-c4cc(F)ccc4C(F)(F)F)cc3C2)n1]

Example 28 was prepared from intermediate I-5 using Suzuki coupling procedure B and (5-fluoro-2-(trifluoromethyl)phenyl)boronic acid. Purification with reversed-phase HPLC (Method G) provided 28 (22% yield) as white solid. ¹H NMR (400 MHz, DMSO-d6) δ 8.90 (d, J=4.6 Hz, 1H), 7.94 (ddd, J=8.1, 5.5, 2.1 Hz, 1H), 7.50 (t, J=7.1 Hz, 2H), 7.39 (s, 1H), 7.32 (t, J=6.1 Hz, 2H), 5.53 (s, 2H), 5.03 (d, J=7.6 Hz, 2H), 4.76 (d, J=11.9 Hz, 2H); LC-MS m/z 416 [M+H]⁺, retention time=1.09 min (Method A).

### Example 29: 1-(2-(5-(4-fluoro-2-(trifluoromethyl)phenyl)isoindolin-2-yl)-2-oxoethyl)-1H-1,2,4-triazole-3-carbonitrile

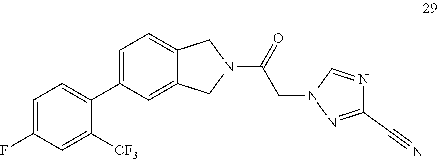 [SMILES: N#Cc1ncn(CC(=O)N2Cc3ccc(-c4ccc(F)cc4C(F)(F)F)cc3C2)n1]

Example 29 was prepared from intermediate I-5 using Suzuki coupling procedure A and (4-fluoro-2-(trifluoromethyl)phenyl)boronic acid. Purification with reversed-phase HPLC (Method F) provided 29 (8% yield) as white solid. ¹H NMR (400 MHz, DMSO-d6) δ 8.89 (d, J=4.7 Hz, 1H), 7.75 (dt, J=9.4, 2.5 Hz, 1H), 7.62 (ddt, J=8.5, 6.1, 2.9 Hz, 1H), 7.48 (dd, J=10.1, 6.5 Hz, 2H), 7.35 (s, 1H), 7.27 (d, J=7.8 Hz, 1H), 5.52 (d, J=2.4 Hz, 2H), 5.02 (d, J=7.1 Hz, 2H), 4.75 (d, J=11.5 Hz, 2H); LC-MS m/z 416 [M+H]⁺, retention time=1.12 min (Method A).

### Example 30: 1-(2-(5-(2-chloro-4,5-difluorophenyl)isoindolin-2-yl)-2-oxoethyl)-1H-1,2,4-triazole-3-carbonitrile

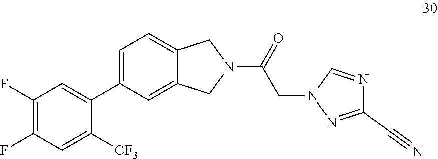 [SMILES: N#Cc1ncn(CC(=O)N2Cc3ccc(-c4cc(F)c(F)cc4C(F)(F)F)cc3C2)n1]

Example 30 was prepared from intermediate I-5 using Suzuki coupling procedure B and (2-chloro-4,5-difluorophenyl)boronic acid. Purification with reversed-phase HPLC (Method I) provided 30 (55% yield) as white solid. ¹H NMR (400 MHz, DMSO-d6) δ 8.90 (d, J=3.0 Hz, 1H), 7.91-7.82 (m, 1H), 7.65-7.56 (m, 1H), 7.54-7.45 (m, 2H), 7.44-7.37 (m, 1H), 5.53 (s, 2H), 5.06-4.98 (m, 2H), 4.76 (d, J=4.7 Hz, 2H); LC-MS m/z 400 [M+H]⁺, retention time=1.10 min (Method A).

### Example 31: 1-(2-(5-(4-methoxy-2-(trifluoromethyl)phenyl)isoindolin-2-yl)-2-oxoethyl)-1H-1,2,4-triazole-3-carbonitrile

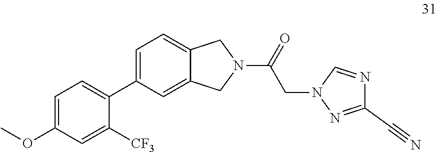 [SMILES: COc1ccc(-c2ccc3c(c2)CN(C(=O)Cn2cnc(C#N)n2)C3)c(C(F)(F)F)c1]

Example 31 was prepared from intermediate I-5 using Suzuki coupling procedure B and (4-methoxy-2-(trifluoromethyl)phenyl)boronic acid. Purification with reversed-phase HPLC (Method I) provided 31 (59% yield) as white solid. ¹H NMR (400 MHz, DMSO-d6) δ 8.95-8.83 (m, 1H), 7.45 (d, J=7.5 Hz, 1H), 7.32-7.24 (m, 5H), 5.60-5.41 (m, 2H), 5.09-4.94 (m, 2H), 4.75 (d, J=9.4 Hz, 2H), 3.89 (s, 3H); LC-MS m/z 428 [M+H]⁺, retention time=1.08 min (Method A).

### Example 32: 1-(2-(5-(2-cyano-4-fluorophenyl)isoindolin-2-yl)-2-oxoethyl)-1H-1,2,4-triazole-3-carbonitrile

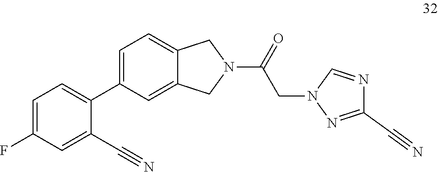 [SMILES: N#Cc1ncn(CC(=O)N2Cc3ccc(-c4ccc(F)cc4C#N)cc3C2)n1]

Example 32 was prepared from intermediate I-5 using Suzuki coupling procedure A and 5-fluoro-2-(4,4,5,5-tetramethyl-1,3,2-dioxaborolan-2-yl)benzonitrile. Purification with reversed-phase HPLC (Method F) provided 32 (9% yield) as white solid. ¹H NMR (400 MHz, DMSO-d6) δ 8.90 (d, J=7.6 Hz, 1H), 8.00-7.97 (m, 1H), 7.77-7.68 (m, 2H), 7.61-7.55 (m, 3H), 5.46 (d, J=2.0 Hz, 2H), 5.04 (s, 2H), 4.77 (d, J=5.6 Hz, 2H); LC-MS m/z 371 [M−H]⁻, retention time=0.96 min (Method A).

### Example 33: 1-(2-(5-(2-chlorophenyl)isoindolin-2-yl)-2-oxoethyl)-1H-1,2,4-triazole-3-carbonitrile

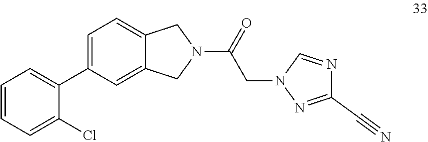 [SMILES: N#Cc1ncn(CC(=O)N2Cc3ccc(-c4ccccc4Cl)cc3C2)n1]

Example 33 was prepared from intermediate I-5 using Suzuki coupling procedure B and (2-chlorophenyl)boronic acid. Purification with reversed-phase HPLC (Method G) provided 33 (26% yield) as white solid. ¹H NMR (400 MHz, DMSO-d6) δ 8.91 (d, J=3.3 Hz, 1H), 7.63-7.56 (m, 1H), 7.52-7.37 (m, 6H), 5.54 (d, J=2.4 Hz, 2H), 5.03 (s, 2H), 4.77 (s, 2H); LC-MS m/z 364 [M+H]⁺, retention time=1.07 min (Method A).

### Example 34: 1-(2-(5-(2-chloro-4-methoxyphenyl)isoindolin-2-yl)-2-oxoethyl)-1H-1,2,4-triazole-3-carbonitrile

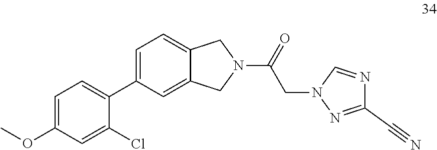 [SMILES: COc1ccc(-c2ccc3c(c2)CN(C(=O)Cn2cnc(C#N)n2)C3)c(Cl)c1]

Example 34 was prepared from intermediate I-5 using Suzuki coupling procedure B and (2-chloro-4-methoxyphenyl)boronic acid. Purification with reversed-phase HPLC (Method I) provided 34 (42% yield) as white solid. ¹H NMR (400 MHz, DMSO-d6) δ 8.91 (d, J=2.8 Hz, 1H), 7.47 (d, J=7.9 Hz, 1H), 7.41 (s, 1H), 7.39-7.32 (m, 2H), 7.16 (t, J=2.6 Hz, 1H), 7.03 (dt, J=8.6, 2.8 Hz, 1H), 5.53 (d, J=2.6 Hz, 2H), 5.02 (s, 2H), 4.75 (s, 2H), 3.88-3.81 (m, 3H); LC-MS m/z 394 [M+H]⁺, retention time=1.06 min (Method A).

### Example 35: 1-(2-(5-(2,6-dichlorophenyl)isoindolin-2-yl)-2-oxoethyl)-1H-1,2,4-triazole-3-carbonitrile

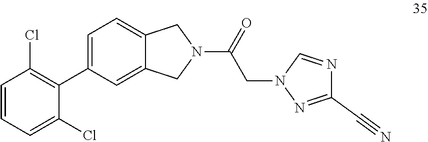 [SMILES: N#Cc1ncn(CC(=O)N2Cc3ccc(-c4c(Cl)cccc4Cl)cc3C2)n1]

Example 35 was prepared from intermediate I-5 using Suzuki coupling procedure A and (2,6-dichlorophenyl)boronic acid. Purification with reversed-phase HPLC (Method F) provided 35 (8% yield) as white solid. ¹H NMR (400 MHz, CDCl₃) δ 8.47 (d, J=2.0 Hz, 1H), 7.43-7.41 (m, 3H), 7.29-7.28 (m, 1H), 7.27-7.22 (m, 2H), 5.17 (d, J=2.4 Hz, 2H), 5.03 (s, 2H), 4.94 (s, 2H); LC-MS m/z 399 [M+H]⁺, retention time=1.11 min (Method A).

### Example 36: 1-(2-oxo-2-(5-(2-(trifluoromethyl)phenyl)isoindolin-2-yl)ethyl)-1H-1,2,4-triazole-3-carbonitrile

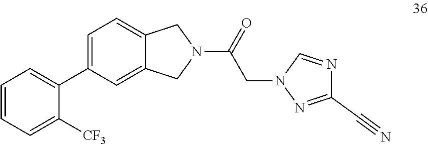 [SMILES: N#Cc1ncn(CC(=O)N2Cc3ccc(-c4ccccc4C(F)(F)F)cc3C2)n1]

Example 36 was prepared from intermediate I-5 using Suzuki coupling procedure A and (2-(trifluoromethyl)phenyl)boronic acid. Purification with reversed-phase HPLC (Method F) provided 36 (8% yield) as white solid. ¹H NMR (400 MHz, DMSO-d6) δ 8.90 (d, J=4.9 Hz, 1H), 7.85 (d, J=8.2 Hz, 1H), 7.74 (dt, J=7.6, 3.6 Hz, 1H), 7.64 (t, J=7.7 Hz, 1H), 7.47 (d, J=7.9 Hz, 1H), 7.45-7.39 (m, 1H), 7.35 (s, 1H), 7.28 (d, J=7.8 Hz, 1H), 5.52 (d, J=3.0 Hz, 2H), 5.02 (d, J=6.4 Hz, 2H), 4.76 (d, J=11.1 Hz, 2H); LC-MS m/z 398 [M+H]⁺, retention time=1.10 min (Method A).

### Example 37: 1-(2-(5-(2-chloro-4-fluorophenyl)isoindolin-2-yl)-2-oxoethyl)-1H-1,2,4-triazole-3-carbonitrile

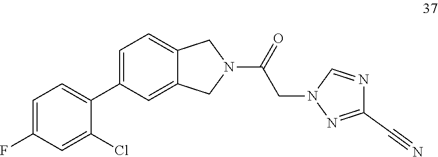 [SMILES: N#Cc1ncn(CC(=O)N2Cc3ccc(-c4ccc(F)cc4Cl)cc3C2)n1]

Example 37 was prepared from intermediate I-5 using Suzuki coupling procedure A and (2-chloro-4-fluorophenyl)boronic acid. Purification with reversed-phase HPLC (Method F) provided 37 (9% yield) as white solid. ¹H NMR (400 MHz, DMSO-d6) δ 8.90 (d, J=3.3 Hz, 1H), 7.58 (dt, J=8.9, 2.6 Hz, 1H), 7.47 (ddd, J=11.3, 9.2, 4.9 Hz, 3H), 7.41-7.29 (m, 2H), 5.53 (d, J=1.7 Hz, 2H), 5.02 (s, 2H), 4.76 (s, 2H); LC-MS m/z 382 [M+H]⁺, retention time=1.10 min (Method A).

### Example 38: 1-(2-(5-(2,4-dichlorophenyl)isoindolin-2-yl)-2-oxoethyl)-1H-1,2,4-triazole-3-carbonitrile

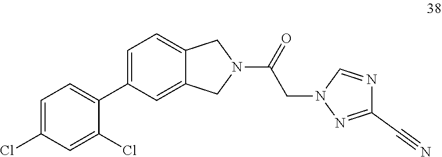 [SMILES: N#Cc1ncn(CC(=O)N2Cc3ccc(-c4ccc(Cl)cc4Cl)cc3C2)n1]

Example 38 was prepared from intermediate I-5 using Suzuki coupling procedure B and (2,4-dichlorophenyl)boronic acid. Purification with reversed-phase HPLC (Method D) provided 41 (31% yield) as white solid. ¹H NMR (400 MHz, DMSO-d6) δ 8.90 (d, J=3.1 Hz, 1H), 7.76 (t, J=2.4 Hz, 1H), 7.57-7.36 (m, 5H), 5.59-5.48 (m, 2H), 5.03 (s, 2H), 4.77 (s, 2H); LC-MS m/z 399 [M+H]⁺, retention time=1.16 min (Method A).

### Example 39: 1-(2-(5-(4-fluoro-2-(2-methoxyethoxy)phenyl)isoindolin-2-yl)-2-oxoethyl)-1H-1,2,4-triazole-3-carbonitrile

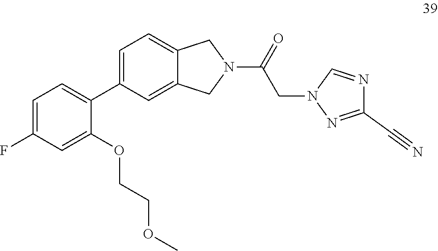 [SMILES: COCCOc1cc(F)ccc1-c1ccc2c(c1)CN(C(=O)Cn1cnc(C#N)n1)C2]

### Preparation of tert-butyl 5-(4-fluoro-2-hydroxyphenyl)isoindoline-2-carboxylate

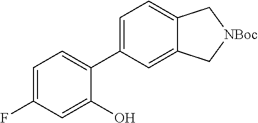 [SMILES: CC(C)(C)OC(=O)N1Cc2ccc(-c3ccc(F)cc3O)cc2C1]

Title compound was prepared from intermediate I-4 using Suzuki coupling procedure H and 2-bromo-5-fluorophenol. The crude compound was purified by column chromatography using 10% EtOAc in n-hexanes, further purified by triturating with pentane to afford title compound, 0.5 g (58.04%, yield). ¹H NMR (400 MHz, Chloroform-d) δ 7.42-7.35 (m, 1H), 7.36-7.30 (m, 2H), 7.17 (m, 1H), 6.76-6.67 (m, 2H), 4.75 (s, 2H), 4.71 (s, 2H), 1.53 (s, 9H); LC-MS m/z 230.2 [M+H-Boc]⁺, retention time=1.77 min (Method B);

### Preparation of tert-butyl 5-(4-fluoro-2-(2-methoxyethoxy)phenyl)isoindoline-2-carboxylate

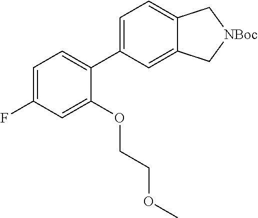 [SMILES: COCCOc1cc(F)ccc1-c1ccc2c(c1)CN(C(=O)OC(C)(C)C)C2]

To a solution of tert-buty 5-(4-fluoro-2-hydroxyphenyl) isoindoline-2-carboxylate (0.1 g, 0.30 mmol) in DMF (5.0 mL) was added potassium carbonate (0.06 g, 0.45 mmol) lot wise at room temperature, then 1-bromo-2-methoxyethane (0.05 g, 0.36 mmol) was added drop wise at room temperature and stirred at 60° C. for 4 h. The reaction mixture diluted with water and extracted with ethylacetate (10 mL×2). The combined organic layer was dried over sodium sulphate and concentrated in vacuo. The crude compound was purified by Column chromatography using 10% EtOAc in n-Hexane to afford title compound as off white solid (0.12 g, 81.53%) Rf=0.5 (20% EtOAc in n-Hexane); ¹H NMR (600 MHz, Chloroform-d) δ 7.49-7.35 (m, 2H), 7.31-7.20 (m, 2H), 6.65-6.69 (m, 2H), 4.71 (s, 2H), 4.67 (s, 2H), 4.09 (t, J=4.7, 4.7 Hz, 2H), 3.70-3.64 (m, 2H), 3.37 (s, 3H), 1.52 (s, 9H); LC-MS m/z 288.3 [M+H-Boc]⁺, retention time=1.92 min (Method B).

### Preparation of 5-(4-fluoro-2-(2-methoxyethoxy)phenyl)isoindoline trifluoroacetic Acid Salt

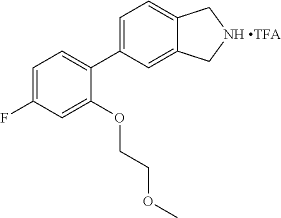

Title compound was prepared from precursor (0.12 g) using Boc-deprotection procedure A. The crude material taken as such for next step (0.15 g). LC-MS m/z 288.3 [M+H]⁺, retention time=0.18 min (Method D); HPLC: 90.74%, retention time=5.45 min (Method E).

### Preparation of 1-(2-(5-(4-fluoro-2-(2-methoxyethoxy)phenyl)isoindolin-2-yl)-2-oxoethyl)-1H-1,2,4-triazole-3-carbonitrile (39)

Compound 39 was prepared from intermediate I-3 using acid amide coupling procedure A. The crude compound was purified by column chromatography using 0-10% methanol in dichloromethane to afford title compound as off white solid 0.04 g (24.27%, yield). ¹H NMR (400 MHz, DMSO-d6): δ 8.90 and 8.89 (s, 1H), 7.52-7.32 (m, 4H), 7.08-7.02 (m, 1H), 6.90-6.84 (m, 1H), 5.53 and 5.52 (m, 2H), 4.98 (s, 2H), 4.72 (s, 2H), 4.18-4.13 (m, 2H), 3.64-3.60 (m, 2H), 3.28 and 3.25 (s, 3H) (NMR shows doubling of protons due to presence of rotamers); LC-MS m/z 421.95 [M+H]⁺, retention time=1.51 min (Method A); HPLC: 98.24%, retention time=6.13 min (Method F).

### Example 40: 1-(2-oxo-2-(5-(2-(trifluoromethoxy)phenyl)isoindolin-2-yl)ethyl)-1H-1,2,4-triazole-3-carbonitrile

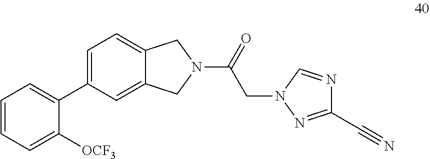 [SMILES: N#Cc1ncn(CC(=O)N2Cc3ccc(-c4ccccc4OC(F)(F)F)cc3C2)n1]

Example 40 was prepared from intermediate I-5 using Suzuki coupling procedure B and (2-(trifluoromethoxy)phenyl)boronic acid. Purification with reversed-phase HPLC (Method I) provided 40 (63% yield) as white solid. ¹H NMR (400 MHz, DMSO-d6) δ 8.90 (d, J=3.9 Hz, 1H), 7.59-7.47 (m, 6H), 7.47-7.41 (m, 1H), 5.53 (s, 2H), 5.04 (s, 2H), 4.77 (d, J=3.2 Hz, 2H); LC-MS m/z 414 [M+H]⁺, retention time=1.10 min (Method A).

### Example 41: 1-(2-(5-(5-cyano-2-fluorophenyl)isoindolin-2-yl)-2-oxoethyl)-1H-1,2,4-triazole-3-carbonitrile

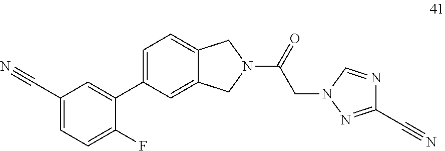 [SMILES: N#Cc1ccc(F)c(-c2ccc3c(c2)CN(C(=O)Cn2cnc(C#N)n2)C3)c1]

Example 41 was prepared from intermediate I-5 using Suzuki coupling procedure A and (5-cyano-2-fluorophenyl)boronic acid. Purification with reversed-phase HPLC (Method F) provided 41 (9% yield) as white solid. ¹H NMR (400 MHz, DMSO-d6) δ 8.90 (d, J=2.0 Hz, 1H), 8.12 (td, J=7.1, 2.1 Hz, 1H), 7.97 (ddt, J=8.6, 4.3, 2.0 Hz, 1H), 7.67-7.50 (m, 4H), 5.54 (d, J=2.0 Hz, 2H), 5.03 (s, 2H), 4.77 (s, 2H); LC-MS m/z 373 [M+H]⁺, retention time=0.95 min (Method A).

### Example 42: 1-(2-(5-(2-chloro-4-(trifluoromethyl)phenyl)isoindolin-2-yl)-2-oxoethyl)-1H-1,2,4-triazole-3-carbonitrile

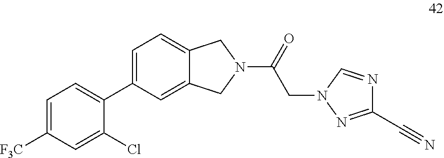 [SMILES: N#Cc1ncn(CC(=O)N2Cc3ccc(-c4ccc(C(F)(F)F)cc4Cl)cc3C2)n1]

Example 42 was prepared from intermediate I-5 using Suzuki coupling procedure B and (2-chloro-4-(trifluoromethyl)phenyl)boronic acid. Purification with reversed-phase HPLC (Method I) provided 42 (52% yield) as white solid. ¹H NMR (400 MHz, DMSO-d6) δ 8.91 (s, 1H), 8.01 (s, 1H), 7.82 (d, J=7.5 Hz, 1H), 7.72-7.61 (m, 1H), 7.49 (dd, J=31.2, 7.6 Hz, 3H), 5.54 (s, 2H), 5.05 (s, 2H), 4.78 (s, 2H); LC-MS m/z 432 [M+H]⁺, retention time=1.17 min (Method A).

### Example 43: 1-(2-(5-(2-cyanophenyl)isoindolin-2-yl)-2-oxoethyl)-1H-1,2,4-triazole-3-carbonitrile

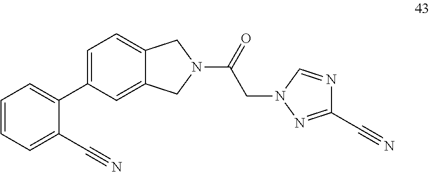 [SMILES: N#Cc1ncn(CC(=O)N2Cc3ccc(-c4ccccc4C#N)cc3C2)n1]

Example 43 was prepared from intermediate I-5 using Suzuki coupling procedure B and (2-cyanophenyl)boronic acid. Purification with reversed-phase HPLC (Method G) provided 43 (17% yield) as white solid. ¹H NMR (400 MHz, DMSO-d6) δ 8.91 (d, J=3.6 Hz, 1H), 7.98 (d, J=7.7 Hz, 1H), 7.87-7.78 (m, 1H), 7.70-7.52 (m, 5H), 5.55 (s, 2H), 5.06 (s, 2H), 4.79 (d, J=3.8 Hz, 2H); LC-MS m/z 355 [M+H]⁺, retention time=0.93 min (Method A).

### Example 44: 1-(2-(5-(3-cyano-2-fluorophenyl)isoindolin-2-yl)-2-oxoethyl)-1H-1,2,4-triazole-3-carbonitrile

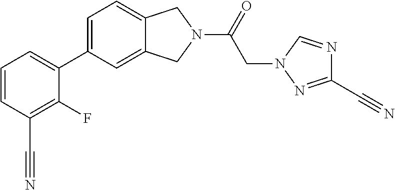 [SMILES: N#Cc1ncn(CC(=O)N2Cc3ccc(-c4cccc(C#N)c4F)cc3C2)n1]

Example 44 was prepared from intermediate I-5 using Suzuki coupling procedure A and (3-cyano-2-fluorophenyl)boronic acid. Purification with reversed-phase HPLC (Method F) provided 44 (9% yield) as white solid. ¹H NMR (400 MHz, DMSO-d6) δ 8.90 (d, J=2.0 Hz, 1H), 8.00-7.85 (m, 2H), 7.63 (s, 1H), 7.59-7.48 (m, 3H), 5.54 (s, 2H), 5.04 (s, 2H), 4.77 (s, 2H); LC-MS m/z 371 [M−H]⁻, retention time=0.95 min (Method A).

### Example 45: 1-(2-oxo-2-(5-(2,4,5-trifluorophenyl)isoindolin-2-yl)ethyl)-1H-1,2,4-triazole-3-carbonitrile

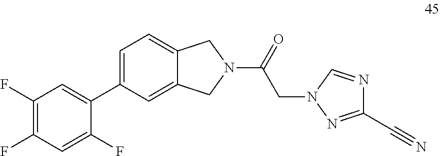 [SMILES: N#Cc1ncn(CC(=O)N2Cc3ccc(-c4cc(F)c(F)cc4F)cc3C2)n1]

Example 45 was prepared from intermediate I-5 using Suzuki coupling procedure C and (2,4,5-trifluorophenyl)boronic acid. Purification with reversed-phase HPLC (Method I) provided 45 (32% yield) as white solid. ¹H NMR (400 MHz, DMSO-d6) δ 8.90 (d, J=1.7 Hz, 1H), 7.79-7.64 (m, 2H), 7.59 (s, 1H), 7.52 (d, J=2.5 Hz, 2H), 5.54 (s, 2H), 5.02 (s, 2H), 4.76 (s, 2H); LC-MS m/z 384 [M+H]⁺, retention time=1.07 min (Method A).

### Example 46: 1-(2-(5-(4-cyanophenyl)isoindolin-2-yl)-2-oxoethyl)-1H-1,2,4-triazole-3-carbonitrile

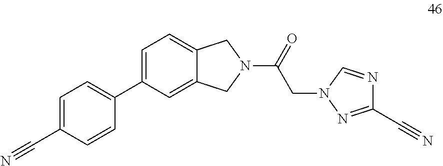 [SMILES: N#Cc1ccc(-c2ccc3c(c2)CN(C(=O)Cn2cnc(C#N)n2)C3)cc1]

Example 46 was prepared from intermediate I-5 using Suzuki coupling procedure C and (4-cyanophenyl)boronic acid. Purification with reversed-phase HPLC (Method G) provided 46 (36% yield) as white solid. ¹H NMR (400 MHz, DMSO-d6) δ 8.90 (d, J=1.4 Hz, 1H), 7.99-7.86 (m, 4H), 7.80 (s, 1H), 7.73 (d, J=8.0 Hz, 1H), 7.58-7.51 (m, 1H), 5.54 (d, J=5.0 Hz, 2H), 5.03 (s, 2H), 4.77 (d, J=5.6 Hz, 2H); LC-MS m/z 355 [M+H]⁺, retention time=0.96 min (Method A).

### Example 47: 1-(2-(5-(2-chloropyridin-3-yl)isoindolin-2-yl)-2-oxoethyl)-1H-1,2,4-triazole-3-carbonitrile

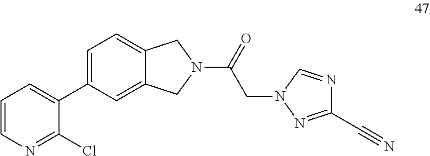 [SMILES: N#Cc1ncn(CC(=O)N2Cc3ccc(-c4cccnc4Cl)cc3C2)n1]

Example 47 was prepared from intermediate I-5 using Suzuki coupling procedure A and (2-chloropyridin-3-yl)boronic acid. Purification with reversed-phase HPLC (Method F) provided 47 (4% yield) as white solid. ¹H NMR (300 MHz, CDCl₃) δ 8.47-8.42 (m, 2H), 7.67 (dd, J=6.9, 2.4 Hz, 1H), 7.44-7.42 (m, 3H), 7.37-7.33 (m, 1H), 5.18 (s, 2H), 5.03 (s, 2H), 4.93 (s, 2H); LC-MS m/z 365 [M+H]⁺, retention time=0.86 min (Method A).

### Example 48: 1-(2-(5-(1-methyl-3-(trifluoromethyl)-1H-pyrazol-5-yl)isoindolin-2-yl)-2-oxoethyl)-1H-1,2,4-triazole-3-carbonitrile

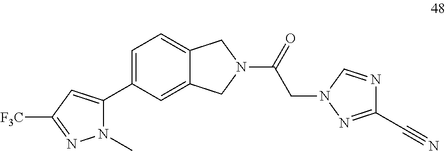 [SMILES: Cn1nc(C(F)(F)F)cc1-c1ccc2c(c1)CN(C(=O)Cn1cnc(C#N)n1)C2]

Example 48 was prepared from intermediate I-5 using Suzuki coupling procedure D and (1-methyl-3-(trifluoromethyl)-1H-pyrazol-5-yl)boronic acid. Purification with reversed-phase HPLC (Method G) provided 48 (44% yield) as white solid. ¹H NMR (400 MHz, DMSO-d6) δ 8.89 (dd, J=7.6, 1.3 Hz, 1H), 8.16 (d, J=6.0 Hz, 1H), 7.70-7.32 (m, 3H), 5.55-5.45 (m, 2H), 5.03-4.90 (m, 2H), 4.76-4.63 (m, 2H), 3.97 (s, 3H); LC-MS m/z 402 [M+H]⁺, retention time=0.98 min (Method A).

### Example 49: 1-(2-(5-(3-chloropyridin-4-yl)isoindolin-2-yl)-2-oxoethyl)-1H-1,2,4-triazole-3-carbonitrile

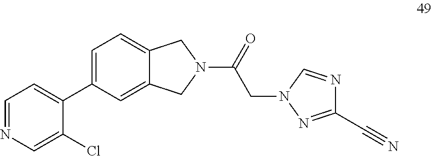 [SMILES: N#Cc1ncn(CC(=O)N2Cc3ccc(-c4ccncc4Cl)cc3C2)n1]

Example 49 was prepared from intermediate I-5 using Suzuki coupling procedure C and (3-chloropyridin-4-yl)boronic acid. Purification with reversed-phase HPLC (Method I) provided 49 (4% yield) as white solid. ¹H NMR (400 MHz, DMSO-d6) δ 8.91 (d, J=3.0 Hz, 1H), 8.76 (d, J=2.2 Hz, 1H), 8.61 (dd, J=4.9, 2.9 Hz, 1H), 7.57 (s, 2H), 7.50 (t, J=5.0 Hz, 2H), 5.54 (s, 2H), 5.04 (s, 2H), 4.78 (s, 2H); LC-MS m/z 365 [M+H]⁺, retention time=0.86 min (Method A).

### Example 50: 1-(2-(5-(2-acetylphenyl)isoindolin-2-yl)-2-oxoethyl)-1H-1,2,4-triazole-3-carbonitrile

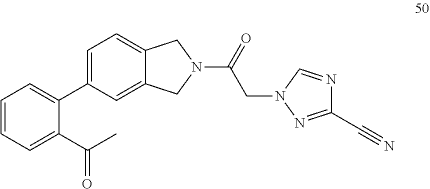 [SMILES: CC(=O)c1ccccc1-c1ccc2c(c1)CN(C(=O)Cn1cnc(C#N)n1)C2]

Example 50 was prepared from intermediate I-5 using Suzuki coupling procedure A and (2-acetylphenyl)boronic acid. Purification with reversed-phase HPLC (Method F) provided 50 (9% yield) as white solid. ¹H NMR (400 MHz, DMSO-d6) δ 8.90 (d, J=3.2 Hz, 1H), 7.61 (dddd, J=16.5, 7.5, 3.1, 1.3 Hz, 2H), 7.53-7.40 (m, 3H), 7.35 (d, J=7.2 Hz, 1H), 7.25 (dq, J=6.2, 1.8 Hz, 1H), 5.53 (d, J=2.2 Hz, 2H), 5.01 (s, 2H), 4.75 (s, 2H), 2.20 (d, J=6.9 Hz, 3H); LC-MS m/z 372 [M+H]⁺, retention time=0.93 min (Method A).

### Example 51: 1-(2-(5-(4-fluoro-2-methoxyphenyl)isoindolin-2-yl)-2-oxoethyl)-1H-1,2,4-triazole-3-carbonitrile

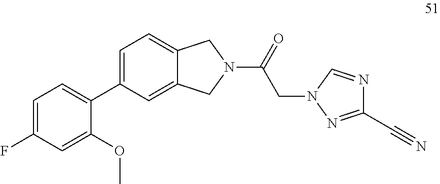 [SMILES: COc1cc(F)ccc1-c1ccc2c(c1)CN(C(=O)Cn1cnc(C#N)n1)C2]

Example 51 was prepared from intermediate I-5 using Suzuki coupling procedure A and (4-fluoro-2-methoxyphenyl)boronic acid. Purification with reversed-phase HPLC (Method F) provided 51 (9% yield) as white solid. ¹H NMR (400 MHz, DMSO-d6) δ 8.90 (d, J=2.1 Hz, 1H), 7.46-7.36 (m, 3H), 7.31 (ddd, J=8.4, 7.0, 5.6 Hz, 1H), 7.04 (dt, J=11.5, 2.9 Hz, 1H), 6.86 (tt, J=8.4, 2.9 Hz, 1H), 5.53 (d, J=2.6 Hz, 2H), 4.99 (s, 2H), 4.73 (s, 2H), 3.78 (d, J=3.1 Hz, 3H); LC-MS m/z 378 [M+H]⁺, retention time=1.04 min (Method A).

### Example 52: 1-(2-(5-(5-chloro-2-methoxyphenyl)isoindolin-2-yl)-2-oxoethyl)-1H-1,2,4-triazole-3-carbonitrile

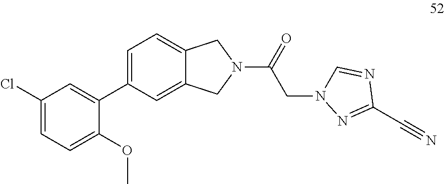 [SMILES: COc1ccc(Cl)cc1-c1ccc2c(c1)CN(C(=O)Cn1cnc(C#N)n1)C2]

Example 52 was prepared from intermediate I-5 using Suzuki coupling procedure A and (5-chloro-2-methoxyphenyl)boronic acid. Purification with reversed-phase HPLC (Method F) provided 52 (8% yield) as white solid. ¹H NMR (400 MHz, DMSO-d6) δ 8.90 (d, J=2.5 Hz, 1H), 7.54-7.47 (m, 1H), 7.47-7.37 (m, 3H), 7.32 (dd, J=6.0, 2.7 Hz, 1H), 7.16 (dd, J=8.9, 2.7 Hz, 1H), 5.53 (s, 2H), 5.00 (s, 2H), 4.74 (s, 2H), 3.77 (d, J=2.6 Hz, 3H); LC-MS m/z 394 [M+H]⁺, retention time=1.11 min (Method A).

### Example 53: 1-(2-(5-(2,4-dimethoxyphenyl)isoindolin-2-yl)-2-oxoethyl)-1H-1,2,4-triazole-3-carbonitrile

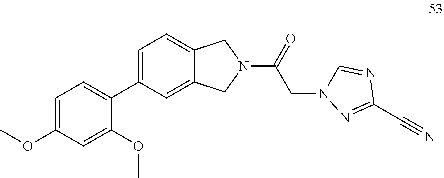 [SMILES: COc1ccc(-c2ccc3c(c2)CN(C(=O)Cn2cnc(C#N)n2)C3)c(OC)c1]

Example 53 was prepared from intermediate I-5 using Suzuki coupling procedure A and (2,4-dimethoxyphenyl)boronic acid. Purification with reversed-phase HPLC (Method A) provided 55 (8% yield) as white solid. ¹H NMR (400 MHz, DMSO-d6) δ 8.90 (d, J=1.7 Hz, 1H), 7.42 (s, 1H), 7.40-7.37 (m, 2H), 7.21 (dd, J=8.4, 4.9 Hz, 1H), 6.70-6.65 (m, 1H), 6.62 (dt, J=8.4, 2.8 Hz, 1H), 5.52 (d, J=3.2 Hz, 2H), 4.98 (s, 2H), 4.72 (s, 2H), 3.81 (d, J=1.8 Hz, 3H), 3.76 (d, J=3.2 Hz, 3H); LC-MS m/z 390 [M+H]⁺, retention time=1.02 min (Method A).

### Example 54: 1-(2-(5-(3-cyanophenyl)isoindolin-2-yl)-2-oxoethyl)-1H-1,2,4-triazole-3-carbonitrile

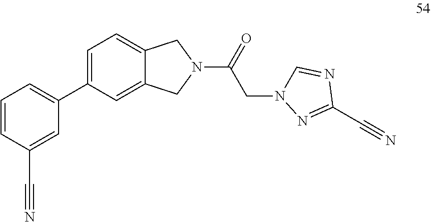 [SMILES: N#Cc1cccc(-c2ccc3c(c2)CN(C(=O)Cn2cnc(C#N)n2)C3)c1]

Example 54 was prepared from intermediate I-5 using Suzuki coupling procedure B and (3-cyanophenyl)boronic acid. Purification with reversed-phase HPLC (Method B) provided 56 (45% yield) as white solid. ¹H NMR (400 MHz, DMSO-d6) δ 8.90 (d, J=1.5 Hz, 1H), 8.18 (dt, J=8.2, 1.5 Hz, 1H), 8.08-8.01 (m, 1H), 7.85 (dq, J=7.7, 1.6 Hz, 1H), 7.80 (s, 1H), 7.76-7.66 (m, 2H), 7.53 (dd, J=8.0, 2.7 Hz, 1H), 5.55 (d, J=7.4 Hz, 2H), 5.03 (s, 2H), 4.77 (d, J=4.1 Hz, 2H); LC-MS m/z 355 [M+H]⁺, retention time=0.94 min (Method A).

### Example 55: 1-(2-(5-(2,4-difluorophenyl)isoindolin-2-yl)-2-oxoethyl)-1H-1,2,4-triazole-3-carbonitrile

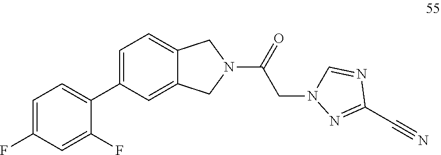 [SMILES: N#Cc1ncn(CC(=O)N2Cc3ccc(-c4ccc(F)cc4F)cc3C2)n1]

Example 57 was prepared from intermediate I-5 using Suzuki coupling procedure A and (2,4-difluorophenyl)boronic acid. Purification with reversed-phase HPLC (Method A) provided 57 (9% yield) as white solid. ¹H NMR (400 MHz, DMSO-d6) δ 8.90 (d, J=1.9 Hz, 1H), 7.64-7.57 (m, 1H), 7.57-7.45 (m, 3H), 7.39 (ddt, J=11.9, 9.4, 2.6 Hz, 1H), 7.22 (tt, J=8.4, 2.2 Hz, 1H), 5.53 (s, 2H), 5.02 (s, 2H), 4.76 (s, 2H); LC-MS m/z 366 [M+H]⁺, retention time=1.05 min (Method A).

### Example 56: 1-(2-(5-(6-fluoropyridin-3-yl)isoindolin-2-yl)-2-oxoethyl)-1H-1,2,4-triazole-3-carbonitrile

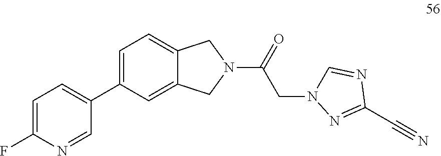 [SMILES: N#Cc1ncn(CC(=O)N2Cc3ccc(-c4ccc(F)nc4)cc3C2)n1]

Example 58 was prepared from intermediate I-5 using Suzuki coupling procedure A and (6-fluoropyridin-3-yl)boronic acid. Purification with reversed-phase HPLC (Method A) provided 58 (9% yield) as white solid. ¹H NMR (300 MHz, DMSO-d6) δ 8.87 (s, 1H), 8.55 (s, 1H), 8.29-8.27 (m, 1H), 7.75 (s, 1H), 7.68 (d, J=7.8 Hz, 1H), 7.51 (d, J=7.8 Hz, 1H), 7.27 (d, J=8.4 Hz, 1H), 5.32 (d, J=3.6 Hz, 2H), 5.01 (s, 2H), 4.76 (d, J=3.9 Hz, 2H); LC-MS m/z 349 [M+H]⁺, retention time=0.86 min (Method A).

### Example 57: 1-(2-(5-(5-fluoro-2-methoxyphenyl)isoindolin-2-yl)-2-oxoethyl)-1H-1,2,4-triazole-3-carbonitrile

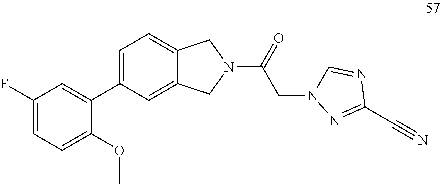 [SMILES: COc1ccc(F)cc1-c1ccc2c(c1)CN(C(=O)Cn1cnc(C#N)n1)C2]

Example 57 was prepared from intermediate I-5 using Suzuki coupling procedure E and (5-fluoro-2-methoxyphenyl)boronic acid. Purification with reversed-phase HPLC (Method B) provided 59 (51% yield) as white solid. ¹H NMR (400 MHz, DMSO-d6) δ 8.90 (d, J=2.3 Hz, 1H), 7.53-7.40 (m, 3H), 7.17 (dddd, J=19.0, 9.2, 5.1, 3.0 Hz, 3H), 5.53 (s, 2H), 5.00 (s, 2H), 4.74 (s, 2H), 3.76 (d, J=3.0 Hz, 3H); LC-MS m/z 378 [M+H]⁺, retention time=1.10 min (Method A).

### Example 58: 1-(2-(5-(4-chloro-2-fluorophenyl)isoindolin-2-yl)-2-oxoethyl)-1H-1,2,4-triazole-3-carbonitrile

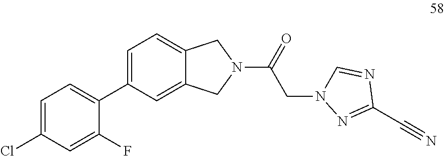 [SMILES: N#Cc1ncn(CC(=O)N2Cc3ccc(-c4ccc(Cl)cc4F)cc3C2)n1]

Example 58 was prepared from intermediate I-5 using Suzuki coupling procedure A and (4-chloro-2-fluorophenyl)boronic acid. Purification with reversed-phase HPLC (Method A) provided 60 (9% yield) as white solid. ¹H NMR (400 MHz, DMSO-d6) δ 8.90 (d, J=1.8 Hz, 1H), 7.57 (ddd, J=7.3, 4.8, 2.6 Hz, 3H), 7.54-7.48 (m, 2H), 7.41 (dt, J=8.3, 2.0 Hz, 1H), 5.53 (s, 2H), 5.02 (s, 2H), 4.76 (s, 2H); LC-MS m/z 382 [M+H]⁺, retention time=1.13 min (Method A).

### Example 59: 1-(2-(5-(2,5-dimethoxyphenyl)isoindolin-2-yl)-2-oxoethyl)-1H-1,2,4-triazole-3-carbonitrile

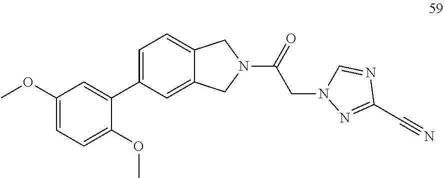 [SMILES: COc1ccc(OC)c(-c2ccc3c(c2)CN(C(=O)Cn2cnc(C#N)n2)C3)c1]

Example 59 was prepared from intermediate I-5 using Suzuki coupling procedure A and (2,5-dimethoxyphenyl)boronic acid. Purification with reversed-phase HPLC (Method A) provided 61 (8% yield) as white solid. ¹H NMR (400 MHz, DMSO-d6) δ 8.90 (d, J=2.0 Hz, 1H), 7.49-7.39 (m, 3H), 7.05 (dd, J=9.0, 3.1 Hz, 1H), 6.92 (dt, J=8.9, 2.7 Hz, 1H), 6.86 (dd, J=4.9, 3.1 Hz, 1H), 5.53 (d, J=2.6 Hz, 2H), 5.00 (s, 2H), 4.73 (s, 2H), 3.75 (d, J=2.4 Hz, 3H), 3.70 (d, J=3.3 Hz, 3H); LC-MS m/z 390 [M+H]⁺, retention time=1.01 min (Method A).

### Example 60: 1-(2-(5-(3-fluoropyridin-4-yl)isoindolin-2-yl)-2-oxoethyl)-1H-1,2,4-triazole-3-carbonitrile

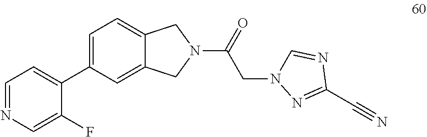 [SMILES: N#Cc1ncn(CC(=O)N2Cc3ccc(-c4ccncc4F)cc3C2)n1]

Example 60 was prepared from intermediate I-5 using Suzuki coupling procedure A and (3-fluoropyridin-4-yl)boronic acid. Purification with reversed-phase HPLC (Method A) provided 62 (9% yield) as white solid. ¹H NMR (400 MHz, CDCl₃) δ 8.57 (t, J=2.0 Hz, 1H), 8.50 (dd, J=5.0, 2.4 Hz, 1H), 8.46 (d, J=1.6 Hz, 1H), 7.59 (d, J=8.4 Hz, 2H), 7.47 (t, J=8.8 Hz, 1H), 7.39 (dd, J=6.8, 4.8 Hz, 1H), 5.18 (s, 2H), 5.04 (d, J=3.6 Hz, 2H), 4.93 (d, J=4.4 Hz, 2H); LC-MS m/z 349 [M+H]⁺, retention time=0.78 min (Method A).

### Example 61: 1-(2-(5-(4-methoxyphenyl)isoindolin-2-yl)-2-oxoethyl)-1H-1,2,4-triazole-3-carbonitrile

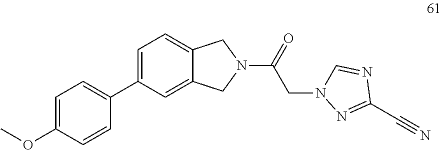 [SMILES: COc1ccc(-c2ccc3c(c2)CN(C(=O)Cn2cnc(C#N)n2)C3)cc1]

Example 61 was prepared from intermediate I-5 using Suzuki coupling procedure A and (4-methoxyphenyl)boronic acid. Purification with reversed-phase HPLC (Method A) provided 63 (9% yield) as white solid. ¹H NMR (400 MHz, DMSO-d6) δ 8.90 (d, J=0.9 Hz, 1H), 7.66-7.55 (m, 4H), 7.45 (d, J=7.9 Hz, 1H), 7.09-6.99 (m, 2H), 5.54 (d, J=2.8 Hz, 2H), 5.00 (d, J=8.3 Hz, 2H), 4.74 (d, J=9.7 Hz, 2H), 3.81 (d, J=1.7 Hz, 3H); LC-MS m/z 360 [M+H]⁺, retention time=1.01 min (Method A).

### Example 62: 1-(2-(5-(2-methoxy-5-(trifluoromethoxy)phenyl)isoindolin-2-yl)-2-oxoethyl)-1H-1,2,4-triazole-3-carbonitrile

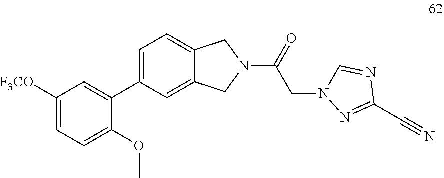 [SMILES: COc1ccc(OC(F)(F)F)cc1-c1ccc2c(c1)CN(C(=O)Cn1cnc(C#N)n1)C2]

Example 62 was prepared from intermediate I-5 using Suzuki coupling procedure A and (2-methoxy-5-(trifluoromethoxy)phenyl)boronic acid. Purification with reversed-phase HPLC (Method A) provided 64 (7% yield) as white solid. ¹H NMR (400 MHz, DMSO-d6) δ 8.91 (d, J=2.1 Hz, 1H), 7.51 (d, J=3.2 Hz, 1H), 7.49-7.34 (m, 3H), 7.32-7.19 (m, 2H), 5.53 (d, J=1.7 Hz, 2H), 5.01 (s, 2H), 4.74 (s, 2H), 3.80 (d, J=2.8 Hz, 3H); LC-MS m/z 444 [M+H]⁺, retention time=1.16 min (Method A).

### Example 63: 1-(2-(5-(2-fluoropyridin-4-yl)isoindolin-2-yl)-2-oxoethyl)-1H-1,2,4-triazole-3-carbonitrile

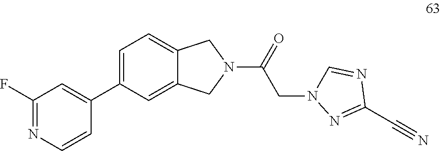 [SMILES: N#Cc1ncn(CC(=O)N2Cc3ccc(-c4ccnc(F)c4)cc3C2)n1]

Example 63 was prepared from intermediate I-5 using Suzuki coupling procedure A and (2-fluoropyridin-4-yl)boronic acid. Purification with reversed-phase HPLC (Method A) provided 65 (9% yield) as white solid. ¹H NMR (400 MHz, DMSO-d6) δ 8.90 (d, J=1.0 Hz, 1H), 8.32 (dd, J=5.3, 1.7 Hz, 1H), 7.92 (s, 1H), 7.85 (d, J=8.0 Hz, 1H), 7.73 (ddt, J=7.2, 3.5, 1.8 Hz, 1H), 7.61-7.49 (m, 2H), 5.54 (d, J=7.8 Hz, 2H), 5.04 (s, 2H), 4.77 (d, J=2.7 Hz, 2H); LC-MS m/z 349 [M+H]⁺, retention time=0.84 min (Method A).

### Example 64: 1-(2-(5-(4-ethynylphenyl)isoindolin-2-yl)-2-oxoethyl)-1H-1,2,4-triazole-3-carbonitrile

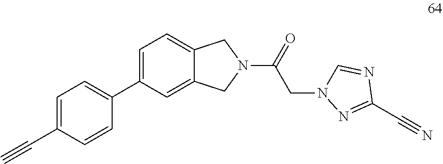 [SMILES: C#Cc1ccc(-c2ccc3c(c2)CN(C(=O)Cn2cnc(C#N)n2)C3)cc1]

Example 64 was prepared from intermediate I-5 using Suzuki coupling procedure A and (4-ethynylphenyl)boronic acid. Purification with reversed-phase HPLC (Method A) provided 66 (8% yield) as white solid. ¹H NMR (400 MHz, DMSO-d6): δ 8.89 (d, J=2.0 Hz, 1H), 7.62-7.43 (m, 7H), 5.52 (d, J=2.8 Hz, 2H), 5.00 (d, J=5.7 Hz, 2H), 4.73 (d, J=4.7 Hz, 2H), 2.55 (s, 1H); LC-MS m/z 354 [M+H]⁺, retention time=1.11 min (Method A).

### Example 65: 1-(2-(5-(2-fluorophenyl)isoindolin-2-yl)-2-oxoethyl)-1H-1,2,4-triazole-3-carbonitrile

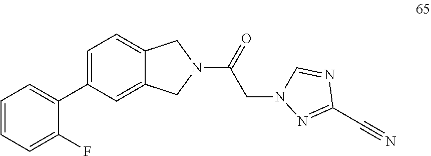 [SMILES: N#Cc1ncn(CC(=O)N2Cc3ccc(-c4ccccc4F)cc3C2)n1]

Example 67 was prepared from intermediate I-5 using Suzuki coupling procedure A and (2-fluorophenyl)boronic acid. Purification with reversed-phase HPLC (Method A) provided 67 (9% yield) as white solid. ¹H NMR (400 MHz, DMSO-d6) δ 8.91 (d, J=1.9 Hz, 1H), 7.60-7.52 (m, 2H), 7.51 (s, 2H), 7.49-7.39 (m, 1H), 7.39-7.28 (m, 2H), 5.54 (s, 2H), 5.03 (s, 2H), 4.76 (s, 2H); LC-MS m/z 348 [M+H]⁺, retention time=1.02 min (Method A).

### Example 66: 1-(2-oxo-2-(5-(pyridin-4-yl)isoindolin-2-yl)ethyl)-1H-1,2,4-triazole-3-carbonitrile

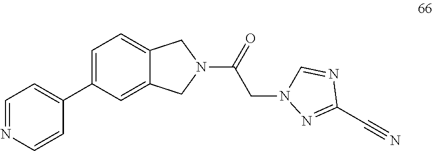 [SMILES: N#Cc1ncn(CC(=O)N2Cc3ccc(-c4ccncc4)cc3C2)n1]

Example 66 was prepared from intermediate I-5 using Suzuki coupling procedure A and pyridin-4-ylboronic acid. Purification with reversed-phase HPLC (Method A) provided 68 (10% yield) as white solid. ¹H NMR (400 MHz, DMSO-d6) δ 8.90 (d, J=0.5 Hz, 1H), 8.70 (s, 2H), 7.89 (s, 1H), 7.82 (d, J=5.6 Hz, 3H), 7.57 (dd, J=8.0, 3.5 Hz, 1H), 5.54 (d, J=5.8 Hz, 2H), 5.04 (d, J=3.4 Hz, 2H), 4.78 (d, J=6.3 Hz, 2H); LC-MS m/z 331 [M+H]⁺, retention time=0.47 min (Method A).

### Example 67: 1-(2-(5-(4-bromophenyl)isoindolin-2-yl)-2-oxoethyl)-1H-1,2,4-triazole-3-carbonitrile

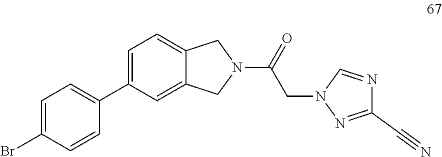 [SMILES: N#Cc1ncn(CC(=O)N2Cc3ccc(-c4ccc(Br)cc4)cc3C2)n1]

Example 67 was prepared from intermediate I-5 using Suzuki coupling procedure A and (4-bromophenyl)boronic acid. Purification with reversed-phase HPLC (Method A) provided 69 (8% yield) as white solid. ¹H NMR (400 MHz, DMSO-d6) δ 8.89 (s, 1H), 7.68-7.65 (m, 6H), 7.52-7.50 (d, J=2.2 Hz, 1H), 5.54 (d, J=3.6 Hz, 2H), 5.0 (d, J=1.8 Hz, 2H), 4.75 (d, J=1.8 Hz, 2H); LC-MS m/z 408, 410 [M+H]⁺, retention time=1.14 min (Method A).

### Example 68: 1-(2-(5-(3-chloro-4-fluorophenyl)isoindolin-2-yl)-2-oxoethyl)-1H-1,2,4-triazole-3-carbonitrile

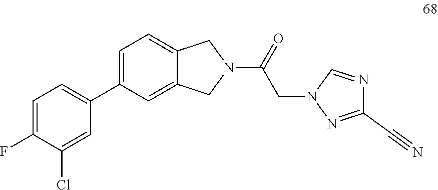 [SMILES: N#Cc1ncn(CC(=O)N2Cc3ccc(-c4ccc(F)c(Cl)c4)cc3C2)n1]

Example 68 was prepared from intermediate I-5 using Suzuki coupling procedure A and (3-chloro-4-fluorophenyl)boronic acid. Purification with reversed-phase HPLC (Method A) provided 70 (9% yield) as white solid. ¹H NMR (400 MHz, DMSO-d6) δ 8.90 (d, J=1.6 Hz, 1H), 7.91 (td, J=7.4, 2.3 Hz, 1H), 7.77-7.62 (m, 3H), 7.56-7.46 (m, 2H), 5.54 (d, J=6.7 Hz, 2H), 5.01 (s, 2H), 4.75 (d, J=4.3 Hz, 2H); LC-MS m/z 382 [M+H]⁺, retention time=1.12 min (Method A).

### Example 69: 1-(2-(5-([1,1′-biphenyl]-2-yl)isoindolin-2-yl)-2-oxoethyl)-1H-1,2,4-triazole-3-carbonitrile

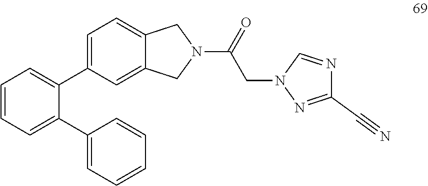 [SMILES: N#Cc1ncn(CC(=O)N2Cc3ccc(-c4ccccc4-c4ccccc4)cc3C2)n1]

Example 69 was prepared from intermediate I-5 using Suzuki coupling procedure A and [1,1′-biphenyl]-2-ylboronic acid. Purification with reversed-phase HPLC (Method A) provided 71 (8% yield) as white solid. ¹H NMR (400 MHz, DMSO-d6) δ 8.87 (d, J=5.5 Hz, 1H), 7.52-7.39 (m, 4H), 7.31-7.19 (m, 5H), 7.17-7.10 (m, 2H), 7.00 (dd, J=19.4, 8.6 Hz, 1H), 5.48 (d, J=3.2 Hz, 2H), 4.89 (d, J=25.7 Hz, 2H), 4.63 (d, J=23.3 Hz, 2H); LC-MS m/z 406 [M+H]⁺, retention time=1.20 min (Method A).

### Example 70: 1-(2-(5-(4-chloro-3-fluorophenyl)isoindolin-2-yl)-2-oxoethyl)-1H-1,2,4-triazole-3-carbonitrile

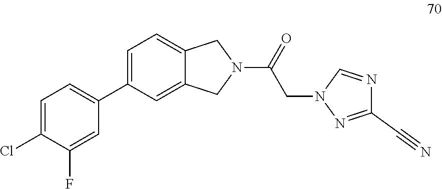 [SMILES: N#Cc1ncn(CC(=O)N2Cc3ccc(-c4ccc(Cl)c(F)c4)cc3C2)n1]

Example 70 was prepared from intermediate I-5 using Suzuki coupling procedure A and (4-chloro-3-fluorophenyl)boronic acid. Purification with reversed-phase HPLC (Method A) provided 72 (9% yield) as white solid. ¹H NMR (400 MHz, DMSO-d6) δ 8.90 (d, J=1.2 Hz, 1H), 7.84-7.75 (m, 2H), 7.69 (ddd, J=9.7, 8.1, 1.5 Hz, 2H), 7.58 (ddd, J=8.3, 6.0, 1.8 Hz, 1H), 7.51 (dd, J=8.1, 2.7 Hz, 1H), 5.54 (d, J=6.4 Hz, 2H), 5.01 (s, 2H), 4.75 (d, J=5.3 Hz, 2H); LC-MS m/z 382 [M+H]⁺, retention time=1.13 min (Method A).

### Example 71: 1-(2-(5-(4-chlorophenyl)isoindolin-2-yl)-2-oxoethyl)-1H-1,2,4-triazole-3-carbonitrile

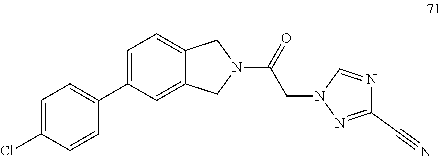 [SMILES: N#Cc1ncn(CC(=O)N2Cc3ccc(-c4ccc(Cl)cc4)cc3C2)n1]

Example 71 was prepared from intermediate I-5 using Suzuki coupling procedure A and (4-chlorophenyl)boronic acid. Purification with reversed-phase HPLC (Method A) provided 73 (9% yield) as white solid. ¹H NMR (400 MHz, DMSO-d6) δ 8.90 (d, J=1.3 Hz, 1H), 7.71 (ddd, J=7.9, 5.1, 2.4 Hz, 3H), 7.64 (d, J=8.0 Hz, 1H), 7.57-7.44 (m, 3H), 5.54 (d, J=3.9 Hz, 2H), 5.01 (d, J=5.9 Hz, 2H), 4.75 (d, J=7.7 Hz, 2H); LC-MS m/z 364 [M+H]⁺, retention time=1.12 min (Method A).

### Example 72: 1-(2-(5-(3,4-difluorophenyl)isoindolin-2-yl)-2-oxoethyl)-1H-1,2,4-triazole-3-carbonitrile

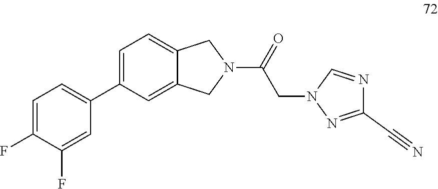 [SMILES: N#Cc1ncn(CC(=O)N2Cc3ccc(-c4ccc(F)c(F)c4)cc3C2)n1]

Example 72 was prepared from intermediate I-5 using Suzuki coupling procedure A and (3,4-difluorophenyl)boronic acid. Purification with reversed-phase HPLC (Method A) provided 74 (9% yield) as white solid. ¹H NMR (400 MHz, DMSO-d6) δ 8.90 (d, J=1.3 Hz, 1H), 7.84-7.74 (m, 1H), 7.73 (s, 1H), 7.67 (d, J=8.1 Hz, 1H), 7.59-7.45 (m, 3H), 5.54 (d, J=6.3 Hz, 2H), 5.01 (d, J=3.4 Hz, 2H), 4.75 (d, J=5.5 Hz, 2H); LC-MS m/z 366 [M+H]⁺, retention time=1.06 min (Method A).

### Example 73: 1-(2-(5-(2-fluoropyridin-3-yl)isoindolin-2-yl)-2-oxoethyl)-1H-1,2,4-triazole-3-carbonitrile

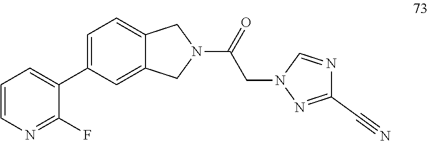 [SMILES: N#Cc1ncn(CC(=O)N2Cc3ccc(-c4cccnc4F)cc3C2)n1]

Example 73 was prepared from intermediate I-5 using Suzuki coupling procedure A and (2-fluoropyridin-3-yl)boronic acid. Purification with reversed-phase HPLC (Method A) provided 75 (9% yield) as white solid. ¹H NMR (400 MHz, DMSO-d6) δ 8.90 (d, J=1.5 Hz, 1H), 8.29-8.23 (m, 1H), 8.18-8.08 (m, 1H), 7.65 (s, 1H), 7.62-7.47 (m, 3H), 5.54 (s, 2H), 5.03 (s, 2H), 4.77 (s, 2H); LC-MS m/z 349 [M+H]⁺, retention time=0.84 min (Method A).

### Example 74: 1-(2-(5-(3,5-difluorophenyl)isoindolin-2-yl)-2-oxoethyl)-1H-1,2,4-triazole-3-carbonitrile

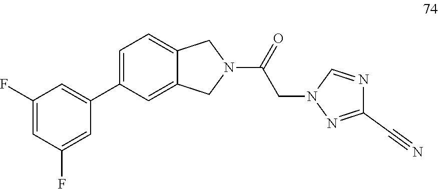 [SMILES: N#Cc1ncn(CC(=O)N2Cc3ccc(-c4cc(F)cc(F)c4)cc3C2)n1]

Example 74 was prepared from intermediate I-5 using Suzuki coupling procedure A and (3,5-difluorophenyl)boronic acid. Purification with reversed-phase HPLC (Method A) provided 76 (9% yield) as white solid. ¹H NMR (400 MHz, DMSO-d6) δ 8.90 (d, J=1.6 Hz, 1H), 7.80 (s, 1H), 7.73 (d, J=8.0 Hz, 1H), 7.49 (tdd, J=10.3, 9.2, 8.7, 2.8 Hz, 3H), 7.26 (ddt, J=9.3, 7.1, 1.8 Hz, 1H), 5.54 (d, J=7.8 Hz, 2H), 5.02 (s, 2H), 4.75 (s, 2H); LC-MS m/z 366 [M+H]⁺, retention time=1.07 min (Method A).

### Example 75: 1-(2-(5-(2-methoxyphenyl)isoindolin-2-yl)-2-oxoethyl)-1H-1,2,4-triazole-3-carbonitrile

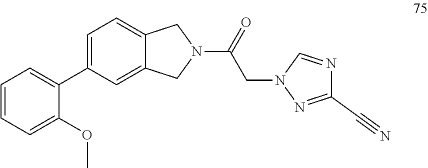 [SMILES: COc1ccccc1-c1ccc2c(c1)CN(C(=O)Cn1cnc(C#N)n1)C2]

Example 75 was prepared from intermediate I-5 using Suzuki coupling procedure A and (2-methoxyphenyl)boronic acid. Purification with reversed-phase HPLC (Method A) provided 75 (9% yield) as white solid. ¹H NMR (400 MHz, DMSO-d6) δ 8.90 (d, J=2.2 Hz, 1H), 7.46 (s, 1H), 7.45-7.40 (m, 2H), 7.40-7.33 (m, 1H), 7.29 (ddd, J=7.3, 5.4, 1.7 Hz, 1H), 7.13 (dd, J=7.5, 3.1 Hz, 1H), 7.04 (tdd, J=7.4, 3.3, 1.1 Hz, 1H), 5.53 (d, J=3.0 Hz, 2H), 5.00 (s, 2H), 4.73 (s, 2H), 3.77 (d, J=3.2 Hz, 3H); LC-MS m/z 360 [M+H]⁺, retention time=1.03 min (Method A).

### Example 76: 1-(2-oxo-2-(5-(4-(trifluoromethyl)phenyl)isoindolin-2-yl)ethyl)-1H-1,2,4-triazole-3-carbonitrile

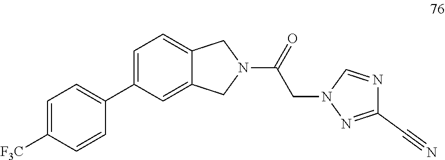 [SMILES: N#Cc1ncn(CC(=O)N2Cc3ccc(-c4ccc(C(F)(F)F)cc4)cc3C2)n1]

Example 76 was prepared from intermediate I-5 using Suzuki coupling procedure B and (4-(trifluoromethyl)phenyl)boronic acid. Purification with reversed-phase HPLC (Method D) provided 78 (58% yield) as white solid. ¹H NMR (400 MHz, DMSO-d6) δ 8.87 (s, 1H), 7.96-7.67 (m, 6H), 7.53 (d, J=7.9 Hz, 1H), 5.52 (s, 2H), 5.02 (d, J=6.2 Hz, 2H), 4.77 (d, J=8.4 Hz, 2H); LC-MS m/z 398 [M+H]⁺, retention time=1.12 min (Method A).

### Example 77: 1-(2-(5-(3-methoxyphenyl)isoindolin-2-yl)-2-oxoethyl)-1H-1,2,4-triazole-3-carbonitrile

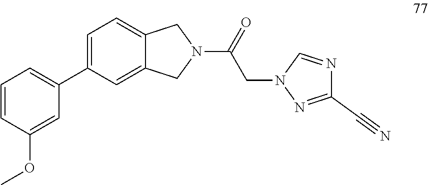 [SMILES: COc1cccc(-c2ccc3c(c2)CN(C(=O)Cn2cnc(C#N)n2)C3)c1]

Example 77 was prepared from intermediate I-5 using Suzuki coupling procedure A and (3-methoxyphenyl)boronic acid. Purification with reversed-phase HPLC (Method A) provided 79 (9% yield) as white solid. ¹H NMR (300 MHz, CD₃OD) δ 8.67 (s, 1H), 7.58 (d, J=7.5 Hz, 2H), 7.42 (d, J=7.5 Hz, 1H), 7.34 (td, J=7.5 Hz, 2.4, 1H), 7.18 (d, J=8.4 Hz, 1H), 7.15-7.14 (m, 1H), 6.97-6.96 (m, 1H), 5.46 (s, 2H), 5.04 (d, J=5.1 Hz, 2H), 4.84 (d, J=7.2 Hz, 2H), 3.84 (d, J=1.2 Hz, 3H); LC-MS m/z 360 [M+H]⁺, retention time=1.02 min (Method A).

### Example 78: 1-(2-(5-(3-methyl-4-(trifluoromethyl)phenyl)isoindolin-2-yl)-2-oxoethyl)-1H-1,2,4-triazole-3-carbonitrile

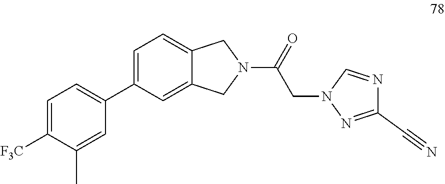 [SMILES: Cc1cc(-c2ccc3c(c2)CN(C(=O)Cn2cnc(C#N)n2)C3)ccc1C(F)(F)F]

Example 78 was prepared from intermediate I-5 using Suzuki coupling procedure A and (3-methyl-4-(trifluoromethyl)phenyl)boronic acid. Purification with reversed-phase HPLC (Method A) provided 80 (8% yield) as white solid. ¹H NMR (400 MHz, DMSO-d6) δ 8.90 (d, J=0.8 Hz, 1H), 7.82-7.72 (m, 3H), 7.70 (d, J=5.5 Hz, 2H), 7.53 (dd, J=8.0, 2.6 Hz, 1H), 5.55 (d, J=4.9 Hz, 2H), 5.03 (d, J=4.1 Hz, 2H), 4.77 (d, J=6.5 Hz, 2H), 2.53 (s, 3H); LC-MS m/z 412 [M+H]⁺, retention time=1.20 min (Method A).

### Example 79: 1-(2-oxo-2-(5-phenylisoindolin-2-yl)ethyl)-1H-1,2,4-triazole-3-carbonitrile

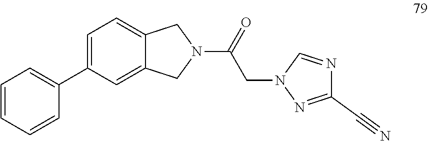 [SMILES: N#Cc1ncn(CC(=O)N2Cc3ccc(-c4ccccc4)cc3C2)n1]

Example 79 was prepared from intermediate I-5 using Suzuki coupling procedure A and phenylboronic acid. Purification with reversed-phase HPLC (Method A) provided 81 (10% yield) as white solid. ¹H NMR (400 MHz, DMSO-d6) δ 8.91 (d, J=1.3 Hz, 1H), 7.72-7.61 (m, 4H), 7.48 (ddd, J=7.8, 5.7, 2.4 Hz, 3H), 7.43-7.34 (m, 1H), 5.54 (d, J=2.9 Hz, 2H), 5.02 (d, J=7.4 Hz, 2H), 4.76 (d, J=8.6 Hz, 2H); LC-MS m/z 330 [M+H]⁺, retention time=1.03 min (Method A).

### Example 80: 1-(2-(5-(3-chlorophenyl)isoindolin-2-yl)-2-oxoethyl)-1H-1,2,4-triazole-3-carbonitrile

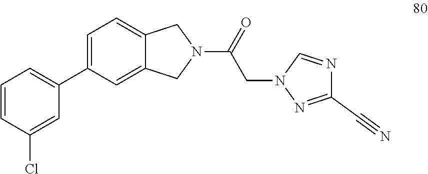 [SMILES: N#Cc1ncn(CC(=O)N2Cc3ccc(-c4cccc(Cl)c4)cc3C2)n1]

Example 80 was prepared from intermediate I-5 using Suzuki coupling procedure A and (3-chlorophenyl)boronic acid. Purification with reversed-phase HPLC (Method A) provided 82 (9% yield) as white solid. ¹H NMR (400 MHz, DMSO-d6) δ 8.90 (d, J=1.8 Hz, 1H), 7.78-7.72 (m, 2H), 7.71-7.60 (m, 2H), 7.55-7.38 (m, 3H), 5.54 (d, J=5.7 Hz, 2H), 5.01 (s, 2H), 4.75 (d, J=4.7 Hz, 2H); LC-MS m/z 364 [M+H]⁺, retention time=1.11 min (Method A).

### Example 81: 1-(2-(5-(3-fluorophenyl)isoindolin-2-yl)-2-oxoethyl)-1H-1,2,4-triazole-3-carbonitrile

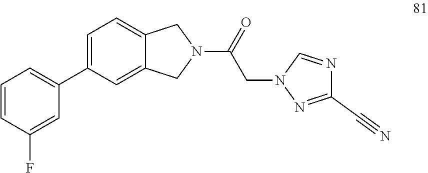 [SMILES: N#Cc1ncn(CC(=O)N2Cc3ccc(-c4cccc(F)c4)cc3C2)n1]

Example 81 was prepared from intermediate I-5 using Suzuki coupling procedure A and (3-fluorophenyl)boronic acid. Purification with reversed-phase HPLC (Method A) provided 83 (9% yield) as white solid. ¹H NMR (400 MHz, DMSO-d6) δ 8.90 (d, J=1.5 Hz, 1H), 7.75 (s, 1H), 7.68 (d, J=8.0 Hz, 1H), 7.58-7.47 (m, 4H), 7.26-7.15 (m, 1H), 5.54 (d, J=5.3 Hz, 2H), 5.02 (d, J=4.4 Hz, 2H), 4.75 (d, J=6.0 Hz, 2H); LC-MS m/z 348 [M+H]⁺, retention time=1.04 min (Method A).

### Example 82: 1-(2-(5-(4-fluorophenyl)isoindolin-2-yl)-2-oxoethyl)-1H-1,2,4-triazole-3-carbonitrile

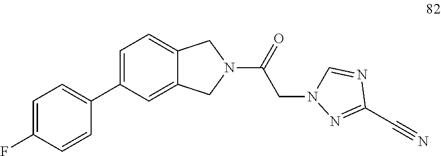 [SMILES: N#Cc1ncn(CC(=O)N2Cc3ccc(-c4ccc(F)cc4)cc3C2)n1]

Example 82 was prepared from intermediate I-5 using Suzuki coupling procedure A and (4-fluorophenyl)boronic acid. Purification with reversed-phase HPLC (Method A) provided 84 (9% yield) as white solid. ¹H NMR (400 MHz, DMSO-d6) δ 8.90 (d, J=1.3 Hz, 1H), 7.77-7.65 (m, 3H), 7.62 (d, J=7.9 Hz, 1H), 7.48 (d, J=6.9 Hz, 1H), 7.31 (tt, J=8.9, 2.3 Hz, 2H), 5.54 (d, J=3.7 Hz, 2H), 5.01 (d, J=6.5 Hz, 2H), 4.75 (d, J=7.9 Hz, 2H); LC-MS m/z 348 [M+H]⁺, retention time=1.03 min (Method A).

### Example 83: 1-(2-oxo-2-(5-(4-(trifluoromethoxy)phenyl)isoindolin-2-yl)ethyl)-1H-1,2,4-triazole-3-carbonitrile

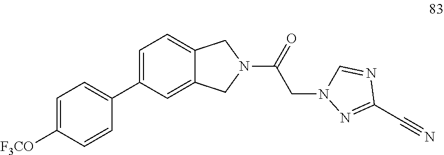 [SMILES: N#Cc1ncn(CC(=O)N2Cc3ccc(-c4ccc(OC(F)(F)F)cc4)cc3C2)n1]

Example 83 was prepared from intermediate I-5 using Suzuki coupling procedure A and (4-(trifluoromethoxy)phenyl)boronic acid. Purification with reversed-phase HPLC (Method A) provided 85 (8% yield) as white solid. ¹H NMR (400 MHz, DMSO-d6) δ 8.90 (d, J=1.3 Hz, 1H), 7.80 (ddd, J=8.5, 5.3, 2.6 Hz, 2H), 7.72 (s, 1H), 7.66 (d, J=8.0 Hz, 1H), 7.55-7.43 (m, 3H), 5.54 (d, J=3.9 Hz, 2H), 5.02 (d, J=6.0 Hz, 2H), 4.76 (d, J=7.3 Hz, 2H); LC-MS m/z 414 [M+H]⁺, retention time=1.16 min (Method A).

### Example 84: 1-(2-(5-(3-fluoro-2-(trifluoromethyl)phenyl)isoindolin-2-yl)-2-oxoethyl)-1H-1,2,4-triazole-3-carbonitrile

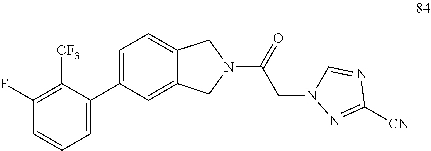 [SMILES: N#Cc1ncn(CC(=O)N2Cc3ccc(-c4cccc(F)c4C(F)(F)F)cc3C2)n1]

### Preparation of tert-butyl 5-(3-chloro-2-(trifluoromethyl)phenyl)isoindoline-2-carboxylate

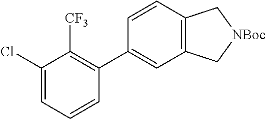 [SMILES: CC(C)(C)OC(=O)N1Cc2ccc(-c3cccc(Cl)c3C(F)(F)F)cc2C1]

Title compound was prepared from 1-bromo-3-chloro-2-(trifluoromethyl)benzene using Suzuki coupling procedure F and boronate ester I-4. Purification with silica gel column chromatography using 0-30% EtOAc in n-hexanes provided product (120 mg, 52.4% yield). ¹H NMR (400 MHz, Chloroform-d) δ 7.58-7.48 (m, 1H), 7.42 (t, J=8.1 Hz, 1H), 7.35-7.19 (m, 2H), 7.20-7.05 (m, 2H), 4.74-4.62 (m, 4H), 1.52 (s, 9H).

### Preparation of 5-(3-chloro-2-(trifluoromethyl)phenyl)isoindoline

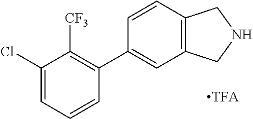 [SMILES: FC(F)(F)c1c(Cl)cccc1-c1ccc2c(c1)CNC2]

Title compound was prepared using Boc-deprotection procedure A. The crude material (100 mg) taken for the next step without purification. LC-MS m/z 297.90 [M+H]⁺, retention time=1.35 min (Method A).

### Preparation of 1-(2-(5-(3-chloro-2-(trifluoromethyl)phenyl)isoindolin-2-yl)-2-oxoethyl)-1H-1,2,4-triazole-3-carbonitrile (84)

Compound 84 was prepared using acid-amine coupling procedure A and acid intermediate I-3. Purification with silica gel column chromatography using 30-100% EtOAc in n-hexanes provided 84 (13 mg, 12.0% yield) as grey solid. ¹H NMR (400 MHz, DMSO-d6): δ 8.89 and 8.88 (s, 1H), 7.78 (d, J=7.6 Hz, 1H), 7.72-7.67 (m, 1H), 7.46 (d, J=8.0 Hz, 1H), 7.36-7.30 (m, 2H), 7.25 (d, J=8.0 Hz, 1H), 5.52 (s, 2H), 5.02 and 4.98 (s, 2H), 4.76-4.73 (s, 2H); (NMR shows doubling of protons due to presence of rotamers); LC-MS m/z 431.90 [M+H]⁺, retention time=1.57 min (Method A); HPLC: 97.10%, retention time=4.95 min (Method C).

### Example 85: 1-(2-oxo-2-(5-(2-(trifluoromethyl)pyridin-3-yl)isoindolin-2-yl)ethyl)-1H-1,2,4-triazole-3-carbonitrile

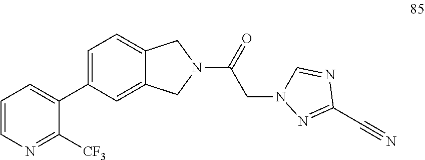 [SMILES: N#Cc1ncn(CC(=O)N2Cc3ccc(-c4cccnc4C(F)(F)F)cc3C2)n1]

### Preparation of 3-(4,4,5,5-tetramethyl-1,3,2-dioxaborolan-2-yl)-2-(trifluoromethyl)pyridine

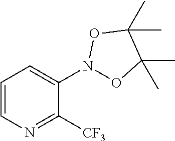 [SMILES: CC1(C)ON(c2cccnc2C(F)(F)F)OC1(C)C]

A 250 ml sealed tube of 3-bromo-2-(trifluoromethyl)pyridine (7.0 g, 30.97 mmol) in dioxane (70 mL) was purged with argon gas for 10 minutes and bispinacalato diborane (8.65 g, 34.07 mmol), KOAc (6.08 g, 61.94 mmol) and Pd(dppf)Cl₂.DCM (1.26 g, 1.54 mmol) were added and the reaction mixture was stirred for 16 h at 100° C. The reaction mixture was diluted with water, extracted with EtOAc (500 mL×2) twice. The combined organic layers were washed with water (300 mL), brine (300 mL), dried over sodium sulphate and concentrated under vacuo. Crude compound was purified by Combiflash chromatography using 40 g Column and 10% EtOAc in n-Hexane to afford title compound as a brownish semisolid 7.0 g (83.0%). Rf=0.50 (10% EtOAc in n-Hexane); 1H NMR (300 MHz, CDCl₃): δ 8.72-8.70 (m, 1H), 8.10-8.00 (m, 1H), 7.50-7.42 (m, 1H), 1.37 (s, 12H); LC-MS: m/z 274.0 (M+H).

### Preparation of tert-butyl 5-(2-(trifluoromethyl) pyridin-3-yl) isoindoline-2-carboxylate

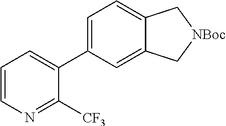 [SMILES: CC(C)(C)OC(=O)N1Cc2ccc(-c3cccnc3C(F)(F)F)cc2C1]

A 250 ml sealed tube with tert-butyl 5-bromoisoindoline-2-carboxylate (7.0 g, 23.48 mmol) in dioxane:H₂O (8:2) (100 mL) was purged with argon gas for 10 minutes. K₃PO₄ (12.44 g, 58.72 mmol), 3-(4,4,5,5-tetramethyl-1,3,2-dioxaborolan-2-yl)-2-(trifluoromethyl)pyridine (7.0 g, 25.82 mmol) and Pd(dppf)Cl₂.DCM (1.92 g, 2.34 mmol) were added and the reaction mixture was stirred for 5 h at 100° C. The reaction mixture was diluted with water, extracted with EtOAc (500 mL×2) twice. The combined organic layers were washed with water (300 mL), brine (300 mL), dried over sodium sulphate and concentrated under vacuo. Crude compound was purified by Combiflash chromatography using 40 g Column and 25% EtOAc in n-Hexane to afford title compound as a brownish semisolid 6.0 g (70.17%). Rf=0.50 (20% EtOAc in n-Hexane); 1H NMR (300 MHz, CDCl₃): δ 8.73 (d, J=3.6 Hz, 1H), 7.72 (d, J=7.8 Hz, 1H), 7.56-7.51 (m, 1H), 7.38-7.28 (m, 1H), 7.25-7.17 (m, 2H), 4.75 (s, 2H), 4.71 (s, 2H), 1.52 (s, 9H).

### Preparation of 5-(2-(trifluoromethyl) pyridin-3-yl) isoindoline. TFA Salt

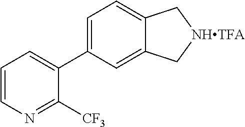

Boc protected precursor (6 g, 16.4 mmol) was dissolved in DCM (20 mL) and trifluoroacetic acid (15 mL) was added at 0° C. The reaction mixture was stirred at rt for 2 h. Reaction mixture was concentrated under vacuo and the crude compound was purified by 10% Diethyl ether in n-Pentane washings to afford title compound as a wheatish solid 7.0 g (Crude). Rf=0.10 (10% MeOH in DCM); LC-MS: [M-TFA]+1=264.9).

### Preparation of 1-(2-oxo-2-(5-(2-(trifluoromethyl) pyridin-3-yl) isoindolin-2-yl) ethyl)-1H-1,2,4-triazole-3-carbonitrile

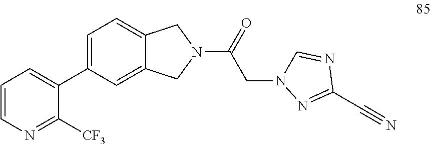 [SMILES: N#Cc1ncn(CC(=O)N2Cc3ccc(-c4cccnc4C(F)(F)F)cc3C2)n1]

To a stirred solution of the TFA salt (7.0 g, 19.33 mmol) and 2-(3-cyano-1H-1,2,4-triazol-1-yl)acetic acid (7.34 g, 48.34 mmol) in DCM (100 mL), DIPEA (12.47 g, 96.68 mmol) and Propyl phosphonic anhydride (T3P) (50% EtOAc solution) (14.5 mL, 23.20 mmol) were added at 0° C. and stirred at rt for 1 h. Reaction mixture was diluted with water, extracted with DCM (500 mL×2) twice. The combined organic layers were washed with water (200 mL), brine (150 mL), dried over sodium sulphate and concentrated under vacuo. Crude compound was purified by Combiflash chromatography using 40 g Column and 50% EtOAc in n-Hexane to afford the title compound as a grey solid 3.1 g (40.28%). Rf=0.4 (70% EtOAc in hexane). Additional purification with reversed-phase HPLC (Method G) provided 85 as grey solid. ¹H NMR (400 MHz, DMSO-d6) δ 8.90 (d, J=4.3 Hz, 1H), 8.80 (d, J=4.6 Hz, 1H), 7.99-7.91 (m, 1H), 7.86-7.77 (m, 1H), 7.52 (d, J=7.8 Hz, 1H), 7.42 (s, 1H), 7.33 (d, J=7.8 Hz, 1H), 5.53 (d, J=1.8 Hz, 2H), 5.04 (d, J=6.4 Hz, 2H), 4.77 (d, J=10.3 Hz, 2H); LC-MS m/z 399 [M+H]⁺, retention time=0.90 min (Method A).

### Example 86: 1-(2-oxo-2-(5-(4-(trifluoromethyl)pyridin-3-yl)isoindolin-2-yl)ethyl)-1H-1,2,4-triazole-3-carbonitrile

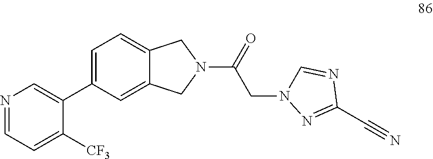 [SMILES: N#Cc1ncn(CC(=O)N2Cc3ccc(-c4cnccc4C(F)(F)F)cc3C2)n1]

Example 86 was prepared from intermediate I-5 using Suzuki coupling procedure B and 3-(4,4,5,5-tetramethyl-1,3,2-dioxaborolan-2-yl)-4-(trifluoromethyl)pyridine. Purification with reversed-phase HPLC (Method H) provided 86 (53% yield) as off-white solid. ¹H NMR (400 MHz, DMSO-d6) δ 8.90 (d, J=4.5 Hz, 2H), 8.71 (d, J=4.6 Hz, 1H), 7.88 (dd, J=5.1, 2.1 Hz, 1H), 7.53 (d, J=7.8 Hz, 1H), 7.45 (s, 1H), 7.36 (d, J=7.8 Hz, 1H), 5.59-5.49 (m, 2H), 5.05 (d, J=6.5 Hz, 2H), 4.78 (d, J=10.9 Hz, 2H); LC-MS m/z 399 [M+H]⁺, retention time=0.89 min (Method A).

### VI. Pharmacology and Utility

Cyanotriazole compounds of the present invention (“cyanotriazole compounds”) showed potent activity against multiple clinical isolates of T. b. gambiense and T. b. rhodesiense. They were also active against melarsoprol and pentamidine resistant mutants of both T. b. brucei and T.b. rhodesiense, indicating they have a novel mode of action compared to standard anti-trypanosomal compounds.

Preliminary morphological investigation using microscopy and flow cytometry of parasites incubated with cyanotrizole compound showed 2 kinetoplastid DNA but single nuclear DNA, suggesting probable defect in nuclear DNA replication or segregation. Since these parasites divide by binary fission, defect in nuclear DNA replication might lead to cytokinesis defect which further led to death of the parasites. The cyanotriazole compounds also showed “concentration and time dependent killing” indicating both increase in concentration of drug and increased duration resulted in increased kill of the parasites. Cyanotriazoles also show ability to sterilize parasites rapidly within 6 hours post treatment under in vitro conditions.

Based on the favorable in vitro and in vivo pharmacological properties, selected cyanotraizole compounds were chosen for testing in stage I and stage II mice models. The stage I mouse model represents the hemolymphatic (blood stream) form of human infection. In this model, all the cyanotraizole compounds tested showed complete cure without relapse at reasonable doses (10 mg/kg QD of Example 8 (1-(2-(5-(5-fluoro-2-(trifluoromethyl)pyridin-3-yl)isoindolin-2-yl)-2-oxoethyl)-1H-1,2,4-triazole-3-carbonitrile), 10 mg/kg BID of Example 29 (1-(2-(5-(4-fluoro-2-(trifluoromethyl)phenyl)isoindolin-2-yl)-2-oxoethyl)-1H-1,2,4-triazole-3-carbonitrile), and 10 mg/kg QD of Example 85 (1-(2-oxo-2-(5-(2-(trifluoromethyl)pyridin-3-yl)isoindolin-2-yl)ethyl)-1H-1,2,4-triazole-3-carbonitrile). The stage II mouse model represents the CNS form of the disease, where in the parasites have invaded the brain similar to human CNS infection. In this model as well, all the compounds tested showed complete cure without relapse (15 mg/kg QD of Example 85, 10 mg/kg QD of Example 8 and 100 mg/kg QD of Example 29).

### VII. Biological Assays

### Parasite Strains and Media

Bloodstream form of T. b. brucei Lister427 strain was obtained from the Genomics Institute of the Novartis Research Foundation. This strain was used for carrying out growth inhibition and kill kinetics assays. T. b. gambiense STIB930 and T. b. rhodesiense STIB900 were obtained from Swiss TPH, were used for carrying out the growth inhibition assays.

Bloodstream form T. b. brucei Lister 427 parasites were continuously passaged in HMI-9 medium formulated from IMDM medium (Invitrogen), 10% heat-inactivated fetal bovine serum (FBS), 10% Serum Plus medium supplement (SAFC Biosciences), 1 mM hypoxanthine (Sigma-Aldrich), 50 μM bathocuproine disulfonic acid (Sigma-Aldrich), 1.5 mM cysteine (Sigma-Aldrich), 1 mM pyruvic acid (Sigma-Aldrich), 39 μg/mL thymidine (Sigma-Aldrich), and 14 μL/L beta-mercapthoethanol (Sigma-Aldrich); all concentrations of added components refer to that in complete HMI-9 medium. The parasites were cultured in 10 mL of HMI-9 medium in T75 CELL-STAR tissue culture flasks at 37° C./5% CO₂.

Bloodstream form of T. b. gambiense and T. b. rhodesiense were also grown in HMI-9 media, described above, but the media was supplemented by 5% human serum and 5% heat-inactivated FBS instead of 10% FBS.

NIH 3T3 fibroblast cells (ATCC) were maintained in RPMI-1640 medium (Life Technologies) supplemented with 10% heat-inactivated fetal bovine serum and 100 IU penicillin/100 μg/ml streptomycin at 37° C./5% CO₂. T. cruzi Tulahuen parasites constitutively expressing E. coli β-galactosidase were maintained in tissue culture as an infection in NIH 3T3 fibroblast cells. Briefly, 2×10⁷ T. cruzi trypomastigotes were used to infect 6×10⁵ NIH 3T3 cells growing in T75 CELLSTAR tissue culture flasks and cultured at 37° C./5% CO₂ until proliferating intracellular parasites lysed host 3T3 cells and were released into the culture medium (typically 6-7 days). During the infection, the tissue culture medium was changed every two days. Number of T. cruzi trypomastigotes present in 1 ml of medium was determined using a haemocytometer.

T. brucei(T. b. brucei and T.b. Gambinese) Growth Inhibition Assay

To determine compound growth inhibitory potency on T. b. brucei Lister427 bloodstream form parasites, 200 nL of 10-point, 3 fold serially diluted compounds in DMSO were transferred to the wells of white, solid bottom 384-well plates (Greiner Bio-One) by either Echo 555 acoustic liquid handling system or Mosquito. Then, 1×10⁴ of T. b. brucei parasites in 40 μL of HMI-9 medium were added to each well, and the plates were incubated for 48 hours at 37° C. in 5% CO₂ incubators. Parasite numbers in individual plate wells were determined through quantification of intracellular ATP amount. The CellTiter-Glo luminescent cell viability reagent (Promega) was added to plate wells, and ATP-dependent luminescence signal was measured on Tecan M1000 plate Reader after 30 min incubation. Luminescence values in wells with compounds were divided by the average luminescence value of the plate DMSO controls, and used for calculation of compound IC₅₀ values.

Similar assay was also used for determining IC₅₀ against T.b. gambinese STIB930 and T. b. rhodesiense STIB900 strains, but the initial concentration of cells used was 3×10⁴ parasites/ml.

T. cruzi Amastigote Growth Inhibition Assay

To determine compound potency on intracellular T. cruzi amastigotes, NIH 3T3 cells were re-suspended in phenol red-free RPMI-1640 medium containing 3% heat-inactivated fetal bovine serum and 100 IU penicillin/100 μg/ml streptomycin, seeded at 1,000 cells/well (40 μl) in white, clear bottom 384-well plates (Greiner Bio-One), and incubated overnight at 37° C./5% CO2. The following day, 100 nl of each compound in DMSO were transferred to individual plate wells by Echo 555 acoustic liquid handling system. After one hour incubation, 1×10⁶ of tissue culture-derived T. cruzi trypomastigotes, in 10 μl of phenol red-free RPMI-1640 medium supplemented with 3% heat-inactivated fetal bovine serum and 100 IU penicillin/100 μg/ml streptomycin were added to each well. Plates were then incubated for 6 days at 37° C./5% CO2. Intracellular T. cruzi parasites were quantified by measuring the activity of parasite-expressed β-galactosidase. Ten microliters of a chromogenic β-galactosidase substrate solution (0.6 mM chlorophenol red-β-D-galactopyranoside/0.6% NP-40 in PBS; both reagents from Calbiochem) were added to each well and incubated for 2 h at room temperature. After incubation, absorption was measured at 570 nM on SpectraMax M2 plate reader (Molecular Devices). Measured absorbance values in wells with compounds were divided by the average absorbance value of the plate DMSO controls, and used for calculation of compound EC₅₀ values as described above.

Assay for Growth Inhibition of Leishmania donovani Axenic Amastigote

Leishmania donovani axenic amastigote parasites are grown at 37° C., 5% CO₂ in media made of RPMI 1640, 4 mM L-glutamine, 20% heat inactivated FBS, 100 units/ml of penicillin and 100 μg/ml of streptomycin, 23 μM Folic Acid, 100 μM Adenosine, 22 mM D-glucose, 25 mM MES. The pH of media is adjusted to 5.5 at 37° C. using HCl. 20 μL of media is first dispensed into 384 well plates and 100 nL of the compounds of invention in DMSO are added to the plate wells. At the same time control compounds and DMSO are added to plates to serve as the positive and negative controls, respectively. 40 μL of parasite culture (9600 parasites) are then added to the plate wells. The plates are then placed into incubators. After 2 day incubation, 20 μL of Cell TiterGlo (Promega) is added to the plate wells. The luminescence signal of each well is measured using the Envision reader (Perkin Elmer). The percentage inhibition of 50%, EC₅₀, is calculated for each of the compounds.

Compounds of the invention have an EC₅₀ of 25 μM or less, typically less than 1 μm, and about half of the compounds have an EC₅₀ below 0.1 μM. Selected compounds of the invention can significantly delay the proliferation of L. donovani. The inhibitory efficacy of the compounds of the invention against L. donovani axenic amastigotes in vitro is provided in Table I.

The exemplified Examples disclosed below were tested in the growth inhibition assays described above and found having anti-parasitic activity against T. b. brucei (T.b.b), T.b. gambiense (T.b.g) T. cruzi (T.c) and L. donovani (L.d). Typically, IC₅₀ values of ≤5 μM (5000 nM) were observed. The resulted IC₅₀ values are summarized in Table 1 below: +≥1 μM; 1 μM>++≥0.1 μM; 0.1 μM>+++

TABLE 1

| | | (a) | (b) | (c) | (d) |
|---|---|---|---|---|---|
| | | T.b.b* | T.b.g* | T.c.* | L.d* |
| Ex | | IC₅₀ | IC₅₀ | IC₅₀ | IC₅₀ |
| # | IUPAC name | (μM) | (μM) | (μM) | (μM) |
| 1 | 1-(2-(5-(2-(difluoromethyl)pyridin- | ++ | | ++ | |
| | 3-yl)isoindolin-2-yl)-2-oxoethyl)- | | | | |
| | 1H-1,2,4-triazole-3-carbonitrile | | | | |
| 2 | 1-(2-(5-(3-chloro-5- | ++ | | +++ | |
| | (trifluoromethyl)pyridin-4- | | | | |
| | yl)isoindolin-2-yl)-2-oxoethyl)-1H- | | | | |
| | 1,2,4-triazole-3-carbonitrile | | | | |
| 3 | 1-(2-(5-(5-chloro-2-fluoropyridin-4- | +++ | | +++ | |
| | yl)isoindolin-2-yl)-2-oxoethyl)-1H- | | | | |
| | 1,2,4-triazole-3-carbonitrile | | | | |
| 4 | 1-(2-(5-(3-chloro-2- | +++ | | +++ | |
| | (trifluoromethyl)phenyl)isoindolin- | | | | |
| | 2-yl)-2-oxoethyl)-1H-1,2,4- | | | | |
| | triazole-3-carbonitrile | | | | |
| 5 | 1-(2-oxo-2-(5-(2-(2,2,2- | ++ | | ++ | |
| | trifluoroethoxy)pyridin-3- | | | | |
| | yl)isoindolin-2-yl)ethyl)-1H-1,2,4- | | | | |
| | triazole-3-carbonitrile | | | | |
| 6 | 1-(2-(5-(6-methyl-2- | +++ | | +++ | |
| | (trifluoromethyl)pyridin-3- | | | | |
| | yl)isoindolin-2-yl)-2-oxoethyl)- | | | | |
| | 1H-1,2,4-triazole-3-carbonitrile | | | | |
| 7 | 1-(2-(5-(2-chloro-4- | ++ | | +++ | |
| | (trifluoromethyl)pyridin-3- | | | | |
| | yl)isoindolin-2-yl)-2-oxoethyl)- | | | | |
| | 1H-1,2,4-triazole-3-carbonitrile | | | | |
| 8 | 1-(2-(5-(5-fluoro-2- | ++ | | ++ | |
| | (trifluoromethyl)pyridin-3- | | | | |
| | yl)isoindolin-2-yl)-2-oxoethyl)- | | | | |
| | 1H-1,2,4-triazole-3-carbonitrile | | | | |
| 9 | 1-(2-(5-(5-fluoro-4- | ++ | | ++ | |
| | (trifluoromethyl)pyridin-3- | | | | |
| | yl)isoindolin-2-yl)-2-oxoethyl)- | | | | |
| | 1H-1,2,4-triazole-3-carbonitrile | | | | |
| 10 | 1-(2-(5-(2-chloro-4,6- | +++ | | +++ | |
| | difluorophenyl)isoindolin-2-yl)-2- | | | | |
| | oxoethyl)-1H-1,2,4-triazole-3- | | | | |
| | carbonitrile | | | | |
| 11 | 1-(2-(5-(4-fluoro-2-(pyrrolidin-1- | ++ | | ++ | |
| | yl)phenyl)isoindolin-2-yl)-2- | | | | |
| | oxoethyl)-1H-1,2,4-triazole-3- | | | | |
| | carbonitrile | | | | |
| 12 | 1-(2-(5-(2-fluoro-6- | +++ | | +++ | |
| | (trifluoromethyl)phenyl)isoindolin- | | | | |
| | 2-yl)-2-oxoethyl)-1H-1,2,4-triazole- | | | | |
| | 3-carbonitrile | | | | |
| 13 | 1-(2-(5-(2-methylpyridin-3- | ++ | | ++ | |
| | yl)isoindolin-2-yl)-2-oxoethyl)-1H- | | | | |
| | 1,2,4-triazole-3-carbonitrile | | | | |
| 14 | 1-(2-(5-(6-methoxy-2- | ++ | | +++ | |
| | (trifluoromethyl)pyridin-3- | | | | |
| | yl)isoindolin-2-yl)-2-oxoethyl)-1H- | | | | |
| | 1,2,4-triazole-3-carbonitrile | | | | |
| 15 | 1-(2-(5-(2-chloro-3,6- | +++ | | +++ | |
| | difluorophenyl)isoindolin-2-yl)-2- | | | | |
| | oxoethyl)-1H-1,2,4-triazole-3- | | | | |
| | carbonitrile | | | | |
| 16 | 1-(2-(5-(3-fluoro-5- | ++ | | +++ | |
| | (trifluoromethyl)pyridin- | | | | |
| | 4-yl)isoindolin-2-yl)-2-oxoethyl)- | | | | |
| | 1H-1,2,4-triazole-3-carbonitrile | | | | |
| 17 | 1-(2-oxo-2-(5-(2- | ++ | | ++ | |
| | (trifluoromethoxy)pyridin- | | | | |
| | 3-yl)isoindolin-2-yl)ethyl)-1H- | | | | |
| | 1,2,4-triazole-3-carbonitrile | | | | |
| 18 | 1-(2-(5-(4-chloro-2- | ++ | | +++ | |
| | (trifluoromethyl)pyridin- | | | | |
| | 3-yl)isoindolin-2-yl)-2-oxoethyl)- | | | | |
| | 1H-1,2,4-triazole-3-carbonitrile | | | | |
| 19 | 1-(2-oxo-2-(5-(2-(2,2,2- | ++ | | ++ | |
| | trifluoroethyl)pyridin-3- | | | | |
| | yl)isoindolin-2-yl)ethyl)-1H- | | | | |
| | 1,2,4-triazole-3-carbonitrile | | | | |
| 20 | 1-(2-(5-(4-fluoro-2- | +++ | | +++ | |
| | (trifluoromethyl)pyridin- | | | | |
| | 3-yl)isoindolin-2-yl)-2-oxoethyl)- | | | | |
| | 1H-1,2,4-triazole-3-carbonitrile | | | | |
| 21 | 1-(2-(5-(2-chloro-4-fluoropyridin- | ++ | | +++ | |
| | 3-yl)isoindolin-2-yl)-2-oxoethyl)- | | | | |
| | 1H-1,2,4-triazole-3-carbonitrile | | | | |
| 22 | 1-(2-(5-(2-fluoro-4- | ++ | | +++ | |
| | (trifluoromethyl)pyridin- | | | | |
| | 3-yl)isoindolin-2-yl)-2-oxoethyl)- | | | | |
| | 1H-1,2,4-triazole-3-carbonitrile | | | | |
| 23 | 1-(2-(5-(3-chloro-5-fluoropyridin- | +++ | | ++ | |
| | 4-yl)isoindolin-2-yl)-2-oxoethyl)- | | | | |
| | 1H-1,2,4-triazole-3-carbonitrile | | | | |
| 24 | 1-(2-(5-(4-chloro-2-fluoropyridin- | ++ | | ++ | |
| | 3-yl)isoindolin-2-yl)-2-oxoethyl)- | | | | |
| | 1H-1,2,4-triazole-3-carbonitrile | | | | |
| 25 | 1-(2-(5-(1-methyl-3- | ++ | | ++ | |
| | (trifluoromethyl)-1H-pyrazol-4- | | | | |
| | yl)isoindolin-2-yl)-2-oxoethyl)- | | | | |
| | 1H-1,2,4-triazole-3-carbonitrile | | | | |
| 26 | 1-(2-(5-(6-fluoro-2- | ++ | | +++ | |
| | (trifluoromethyl)pyridin- | | | | |
| | 3-yl)isoindolin-2-yl)-2-oxoethyl)- | | | | |
| | 1H-1,2,4-triazole-3-carbonitrile | | | | |
| 27 | 1-(2-oxo-2-(5-(3- | +++ | +++ | +++ | |
| | (trifluoromethyl)pyridin-4- | | | | |
| | yl)isoindolin-2-yl)ethyl)-1H- | | | | |
| | 1,2,4-triazole-3-carbonitrile | | | | |
| 28 | 1-(2-(5-(5-fluoro-2- | +++ | | | |
| | (trifluoromethyl)phenyl)isoindolin- | | | | |
| | 2-yl)-2-oxoethyl)-1H-1,2,4- | | | | |
| | triazole-3-carbonitrile | | | | |
| 29 | 1-(2-(5-(4-fluoro-2- | +++ | ++ | +++ | + |
| | (trifluoromethyl)phenyl)isoindolin- | | | | |
| | 2-yl)-2-oxoethyl)-1H-1,2,4- | | | | |
| | triazole-3-carbonitrile | | | | |
| 30 | 1-(2-(5-(2-chloro-4,5- | +++ | ++ | +++ | + |
| | difluorophenyl)isoindolin-2-yl)-2- | | | | |
| | oxoethyl)-1H-1,2,4-triazole-3- | | | | |
| | carbonitrile | | | | |
| 31 | 1-(2-(5-(4-methoxy-2- | +++ | ++ | +++ | + |
| | (trifluoromethyl)phenyl)isoindolin- | | | | |
| | 2-yl)-2-oxoethyl)-1H-1,2,4- | | | | |
| | triazole-3-carbonitrile | | | | |
| 32 | 1-(2-(5-(2-cyano-4- | ++ | | +++ | + |
| | fluorophenyl)isoindolin- | | | | |
| | 2-yl)-2-oxoethyl)-1H-1,2,4- | | | | |
| | triazole-3-carbonitrile | | | | |
| 33 | 1-(2-(5-(2-chlorophenyl)isoindolin- | ++ | ++ | +++ | + |
| | 2-yl)-2-oxoethyl)-1H-1,2,4- | | | | |
| | triazole-3-carbonitrile | | | | |
| 34 | 1-(2-(5-(2-chloro-4- | ++ | ++ | +++ | + |
| | methoxyphenyl)isoindolin-2-yl)- | | | | |
| | 2-oxoethyl)-1H-1,2,4-triazole-3- | | | | |
| | carbonitrile | | | | |
| 35 | 1-(2-(5-(2,6- | ++ | | +++ | + |
| | dichlorophenyl)isoindolin- | | | | |
| | 2-yl)-2-oxoethyl)-1H-1,2,4- | | | | |
| | triazole-3-carbonitrile | | | | |
| 36 | 1-(2-oxo-2-(5-(2- | ++ | | +++ | + |
| | (trifluoromethyl)phenyl)isoindolin- | | | | |
| | 2-yl)ethyl)-1H-1,2,4-triazole-3- | | | | |
| | carbonitrile | | | | |
| 37 | 1-(2-(5-(2-chloro-4- | ++ | ++ | +++ | |
| | fluorophenyl)isoindolin- | | | | |
| | 2-yl)-2-oxoethyl)-1H-1,2,4- | | | | |
| | triazole-3-carbonitrile | | | | |
| 38 | 1-(2-(5-(2,4- | ++ | ++ | +++ | + |
| | dichlorophenyl)isoindolin- | | | | |
| | 2-yl)-2-oxoethyl)-1H-1,2,4- | | | | |
| | triazole-3-carbonitrile | | | | |
| 39 | 1-(2-(5-(4-fluoro-2-(2- | ++ | | + | |
| | methoxyethoxy)phenyl)isoindolin- | | | | |
| | 2-yl)-2-oxoethyl)-1H-1,2,4- | | | | |
| | triazole-3-carbonitrile | | | | |
| 40 | 1-(2-oxo-2-(5-(2- | ++ | | ++ | |
| | (trifluoromethoxy)phenyl)isoindolin- | | | | |
| | 2-yl)ethyl)-1H-1,2,4-triazole-3- | | | | |
| | carbonitrile | | | | |
| 41 | 1-(2-(5-(5-cyano-2- | ++ | ++ | +++ | + |
| | fluorophenyl)isoindolin- | | | | |
| | 2-yl)-2-oxoethyl)-1H-1,2,4-triazole- | | | | |
| | 3-carbonitrile | | | | |
| 42 | 1-(2-(5-(2-chloro-4- | ++ | | + | |
| | (trifluoromethyl)phenyl)isoindolin-2- | | | | |
| | yl)-2-oxoethyl)-1H-1,2,4-triazole-3- | | | | |
| | carbonitrile | | | | |
| 43 | 1-(2-(5-(2-cyanophenyl)isoindolin-2- | ++ | | +++ | + |
| | yl)-2-oxoethyl)-1H-1,2,4-triazole-3- | | | | |
| | carbonitrile | | | | |
| 44 | 1-(2-(5-(3-cyano-2- | ++ | | ++ | |
| | fluorophenyl)isoindolin-2- | | | | |
| | yl)-2-oxoethyl)-1H-1,2,4-triazole- | | | | |
| | 3-carbonitrile | | | | |
| 45 | 1-(2-oxo-2-(5-(2,4,5- | ++ | ++ | ++ | |
| | trifluorophenyl)isoindolin-2-yl)ethyl)- | | | | |
| | 1H-1,2,4-triazole-3-carbonitrile | | | | |
| 46 | 1-(2-(5-(4-cyanophenyl)isoindolin-2- | ++ | | ++ | + |
| | yl)-2-oxoethyl)-1H-1,2,4-triazole-3- | | | | |
| | carbonitrile | | | | |
| 47 | 1-(2-(5-(2-chloropyridin-3- | ++ | | | |
| | yl)isoindolin-2-yl)-2-oxoethyl)-1H- | | | | |
| | 1,2,4-triazole-3-carbonitrile | | | | |
| 48 | 1-(2-(5-(1-methyl-3-(trifluoromethyl)- | ++ | | ++ | |
| | 1H-pyrazol-5-yl)isoindolin-2-yl)-2- | | | | |
| | oxoethyl)-1H-1,2,4-triazole-3- | | | | |
| | carbonitrile | | | | |
| 49 | 1-(2-(5-(3-chloropyridin-4- | ++ | | | |
| | yl)isoindolin-2-yl)-2-oxoethyl)-1H- | | | | |
| | 1,2,4-triazole-3-carbonitrile | | | | |
| 50 | 1-(2-(5-(2-acetylphenyl)isoindolin-2- | ++ | | ++ | |
| | yl)-2-oxoethyl)-1H-1,2,4-triazole-3- | | | | |
| | carbonitrile | | | | |
| 51 | 1-(2-(5-(4-fluoro-2- | ++ | | ++ | |
| | methoxyphenyl)isoindolin-2-yl)-2- | | | | |
| | oxoethyl)-1H-1,2,4-triazole-3- | | | | |
| | carbonitrile | | | | |
| 52 | 1-(2-(5-(5-chloro-2- | ++ | | ++ | |
| | methoxyphenyl)isoindolin-2-yl)-2- | | | | |
| | oxoethyl)-1H-1,2,4-triazole-3- | | | | |
| | carbonitrile | | | | |
| 53 | 1-(2-(5-(2,4- | + | | | |
| | dimethoxyphenyl)isoindolin- | | | | |
| | 2-yl)-2-oxoethyl)-1H-1,2,4-triazole- | | | | |
| | 3-carbonitrile | | | | |
| 54 | 1-(2-(5-(3-cyanophenyl)isoindolin-2- | + | | | |
| | yl)-2-oxoethyl)-1H-1,2,4-triazole-3- | | | | |
| | carbonitrile | | | | |
| 55 | 1-(2-(5-(2,4-difluorophenyl)isoindolin- | + | | | |
| | 2-yl)-2-oxoethyl)-1H-1,2,4-triazole-3- | | | | |
| | carbonitrile | | | | |
| 56 | 1-(2-(5-(6-fluoropyridin-3- | + | | | |
| | yl)isoindolin-2-yl)-2-oxoethyl)-1H- | | | | |
| | 1,2,4-triazole-3-carbonitrile | | | | |
| 57 | 1-(2-(5-(5-fluoro-2- | + | | | |
| | methoxyphenyl)isoindolin-2-yl)- | | | | |
| | 2-oxoethyl)-1H-1,2,4-triazole-3- | | | | |
| | carbonitrile | | | | |
| 58 | 1-(2-(5-(4-chloro-2- | + | | | |
| | fluorophenyl)isoindolin- | | | | |
| | 2-yl)-2-oxoethyl)-1H-1,2,4- | | | | |
| | triazole-3-carbonitrile | | | | |
| 59 | 1-(2-(5-(2,5- | + | | | |
| | dimethoxyphenyl)isoindolin-2- | | | | |
| | yl)-2-oxoethyl)-1H-1,2,4- | | | | |
| | triazole-3-carbonitrile | | | | |
| 60 | 1-(2-(5-(3-fluoropyridin-4- | + | | | |
| | yl)isoindolin-2-yl)-2- | | | | |
| | oxoethyl)-1H-1,2,4-triazole-3- | | | | |
| | carbonitrile | | | | |
| 61 | 1-(2-(5-(4-methoxyphenyl)isoindolin- | + | | | |
| | 2-yl)-2-oxoethyl)-1H-1,2,4-triazole-3- | | | | |
| | carbonitrile | | | | |
| 62 | 1-(2-(5-(2-methoxy-5- | + | | | |
| | (trifluoromethoxy)phenyl)isoindolin- | | | | |
| | 2-yl)-2-oxoethyl)-1H-1,2,4-triazole-3- | | | | |
| | carbonitrile | | | | |
| 63 | 1-(2-(5-(2-fluoropyridin-4- | + | | | |
| | yl)isoindolin-2-yl)-2-oxoethyl)-1H- | | | | |
| | 1,2,4-triazole-3-carbonitrile | | | | |
| 64 | 1-(2-(5-(4-ethynylphenyl)isoindolin- | + | | | |
| | 2-yl)-2-oxoethyl)-1H-1,2,4-triazole-3- | | | | |
| | carbonitrile | | | | |
| 65 | 1-(2-(5-(2-fluorophenyl)isoindolin-2- | + | | | |
| | yl)-2-oxoethyl)-1H-1,2,4-triazole-3- | | | | |
| | carbonitrile | | | | |
| 66 | 1-(2-oxo-2-(5-(pyridin-4-yl)isoindolin- | + | | | |
| | 2-yl)ethyl)-1H-1,2,4-triazole-3- | | | | |
| | carbonitrile | | | | |
| 67 | 1-(2-(5-(4-bromophenyl)isoindolin-2- | + | | | |
| | yl)-2-oxoethyl)-1H-1,2,4-triazole-3- | | | | |
| | carbonitrile | | | | |
| 68 | 1-(2-(5-(3-chloro-4- | + | | | |
| | fluorophenyl)isoindolin- | | | | |
| | 2-yl)-2-oxoethyl)-1H-1,2,4- | | | | |
| | triazole-3-carbonitrile | | | | |
| 69 | 1-(2-(5-([1,1′-biphenyl]-2- | + | | | |
| | yl)isoindolin-2-yl)-2-oxoethyl)-1H- | | | | |
| | 1,2,4-triazole-3-carbonitrile | | | | |
| 70 | 1-(2-(5-(4-chloro-3- | + | | | |
| | fluorophenyl)isoindolin- | | | | |
| | 2-yl)-2-oxoethyl)-1H-1,2,4- | | | | |
| | triazole-3-carbonitrile | | | | |
| 71 | 1-(2-(5-(4-chlorophenyl)isoindolin-2- | + | | | |
| | yl)-2-oxoethyl)-1H-1,2,4-triazole-3- | | | | |
| | carbonitrile | | | | |
| 72 | 1-(2-(5-(3,4-difluorophenyl)isoindolin- | + | | | |
| | 2-yl)-2-oxoethyl)-1H-1,2,4-triazole-3- | | | | |
| | carbonitrile | | | | |
| 73 | 1-(2-(5-(2-fluoropyridin-3- | + | | | |
| | yl)isoindolin-2-yl)-2-oxoethyl)-1H- | | | | |
| | 1,2,4-triazole-3-carbonitrile | | | | |
| 74 | 1-(2-(5-(3,5-difluorophenyl)isoindolin- | + | | | |
| | 2-yl)-2-oxoethyl)-1H-1,2,4-triazole-3- | | | | |
| | carbonitrile | | | | |
| 75 | 1-(2-(5-(2-methoxyphenyl)isoindolin- | + | | | |
| | 2-yl)-2-oxoethyl)-1H-1,2,4-triazole-3- | | | | |
| | carbonitrile | | | | |
| 76 | 1-(2-oxo-2-(5-(4- | + | | | |
| | (trifluoromethyl)phenyl)isoindolin-2- | | | | |
| | yl)ethyl)-1H-1,2,4-triazole-3- | | | | |
| | carbonitrile | | | | |
| 77 | 1-(2-(5-(3-methoxyphenyl)isoindolin- | + | | | |
| | 2-yl)-2-oxoethyl)-1H-1,2,4-triazole-3- | | | | |
| | carbonitrile | | | | |
| 78 | 1-(2-(5-(3-methyl-4- | + | | | |
| | (trifluoromethyl)phenyl)isoindolin-2- | | | | |
| | yl)-2-oxoethyl)-1H-1,2,4-triazole-3- | | | | |
| | carbonitrile | | | | |
| 79 | 1-(2-oxo-2-(5-phenylisoindolin-2- | + | | | |
| | yl)ethyl)-1H-1,2,4-triazole-3- | | | | |
| | carbonitrile | | | | |
| 80 | 1-(2-(5-(3-chlorophenyl)isoindolin- | + | | | |
| | 2-yl)-2-oxoethyl)-1H-1,2,4-triazole- | | | | |
| | 3-carbonitrile | | | | |
| 81 | 1-(2-(5-(3-fluorophenyl)isoindolin- | + | | | |
| | 2-yl)-2-oxoethyl)-1H-1,2,4-triazole- | | | | |
| | 3-carbonitrile | | | | |
| 82 | 1-(2-(5-(4-fluorophenyl)isoindolin-2- | + | | | |
| | yl)-2-oxoethyl)-1H-1,2,4-triazole-3- | | | | |
| | carbonitrile | | | | |
| 83 | 1-(2-oxo-2-(5-(4- | + | | | |
| | (trifluoromethoxy)phenyl)isoindolin- | | | | |
| | 2-yl)ethyl)-1H-1,2,4-triazole-3- | | | | |
| | carbonitrile | | | | |
| 84 | 1-(2-(5-(3-fluoro-2- | ++ | | +++ | |
| | (trifluoromethyl)phenyl)isoindolin-2- | | | | |
| | yl)-2-oxoethyl)-1H-1,2,4-triazole-3- | | | | |
| | carbonitrile | | | | |
| 85 | 1-(2-oxo-2-(5-(2- | +++ | +++ | +++ | + |
| | (trifluoromethyl)pyridin-3- | | | | |
| | yl)isoindolin-2-yl)ethyl)-1H-1,2,4- | | | | |
| | triazole-3-carbonitrile | | | | |
| 86 | 1-(2-oxo-2-(5-(4- | ++ | | ++ | |
| | (trifluoromethyl)pyridin-3- | | | | |
| | yl)isoindolin-2-yl)ethyl)-1H-1,2,4- | | | | |
| | triazole-3-carbonitrile | | | | |

* T.b.b is T. b. brucei , T.b.g. is T.b. gambiense and T.c. is T. cruzi

Accordingly, the compounds of the present invention have been found to inhibit growth of kinetoplastids and therefore useful in the treatment of diseases or disorders associated with kinetoplastids, which include, but are not limited to, human Africa typanosomiasis and Chagas disease.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A compound of Formula (I): 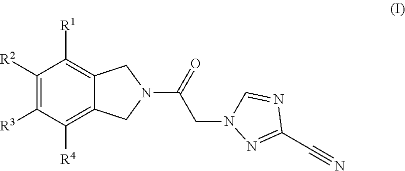
or a pharmaceutically acceptable salt thereof, wherein
R¹, R² and R⁴ are independently H, halogen or C₁-C₄ alkyl;
R³ is independently selected from phenyl and a 5- to 6-membered heteroaryl comprising carbon atoms and 1-4 heteroatoms selected from N, NR^{a}, O, and S(O)ₚ; wherein said phenyl and heteroaryl are substituted with 0-4 R^{3A};
each R^{3A} is independently selected from halogen, CN, OH, C₁-C₆ alkyl, C₂-C₆ alkenyl, C₂-C₆ alkynyl, C₁-C₆ alkoxy, C₁-C₆ haloalkyl, C₁-C₆ haloalkoxy, C(O)—C₁-C₄ alkyl, and phenyl;
each R^{a} is independently selected from H and C₁-C₄ alkyl; and
each p is independently selected from 0, 1 and 2.

2. The compound of claim 1, or a pharmaceutically acceptable salt thereof, wherein
R³ is phenyl.

3. The compound of claim 2, or a pharmaceutically acceptable salt thereof, wherein
R³ is selected from Ph, 2-F-Ph, 3-F-Ph, 4-F-Ph, 2-Cl-Ph, 3-Cl-Ph, 4-Cl-Ph, 4-Br-Ph, 3-CF₃-Ph, 4-CF₃-Ph, 2-OMe-Ph, 3-OMe-Ph, 4-OMe-Ph, 2-OCF₃-Ph, 4-OCF₃-Ph, 2-CN-Ph, 3-CN-Ph, 4-CN-Ph, 2-C(O)Me-Ph, 1,1′-biphenyl-2-yl, 3,4-diF-Ph, 3,5-diF-Ph, 2-F-4-Cl-Ph, 3-F-4-Cl-Ph, 2-Cl-4-F-Ph, 3-Cl-4-F-Ph, 2,4-diCl-Ph, 2-CF₃-4-F-Ph, 2-CF₃-5-F-Ph, 2-CN-4-F-Ph, 2-F-3-CN-Ph, 2-F-5-CN-Ph, 2-CN-4-F-Ph, 2-OMe-4-F-Ph, 2-OMe-5-F-Ph, 2-Cl-4-OMe-Ph, 2-OMe-5-Cl-Ph, 2-OMe-4-OMe-Ph, 2-OMe-5-OMe-Ph, 3-Me-4-CF₃-Ph, 2-CF₃-4-F-Ph, 2-CF₃-4-OMe-Ph, 2-OMe-5-OCF₃-Ph, 2,4,5-triF-Ph, 2-Cl-4,5-diF-Ph, 2,6-diCl-Ph, 2-CF₃-Ph, 2-Cl-4-CF₃-Ph, 2,4-diF-Ph, and 4-ethynyl-Ph, wherein Ph represents phenyl.

4. The compound of claim 1, or a pharmaceutically acceptable salt thereof, wherein
R³ is pyridinyl.

5. The compound of claim 4, or a pharmaceutically acceptable salt thereof, wherein
R³ is selected from pyrid-4-yl, 2-F-pyrid-3-yl, 6-F-pyrid-3-yl, 2-F-pyrid-4-yl, 3-F-pyrid-4-yl, 2-Cl-pyrid-3-yl, 2-Cl-pyrid-4-yl, 2-CF₃-pyrid-4-yl, 3-Cl-pyrid-4-yl, 3-CF₃-pyrid-4-yl, 2-CF₃-pyrid-3-yl, and 4-CF₃-pyrid-3-yl.

6. The compound of claim 1, or a pharmaceutically acceptable salt thereof, wherein
at least one of R¹, R² or R³ is H.

7. The compound of claim 1, wherein the compound is of Formula IA: 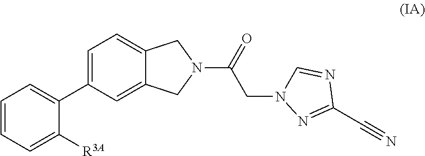
or a pharmaceutically acceptable salt thereof.

8. The compound of claim 1, wherein the compound is of Formula (IB): 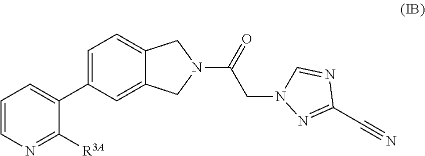
or a pharmaceutically acceptable salt thereof.

9. The compound of claim 1, wherein the compound is of Formula (IC): 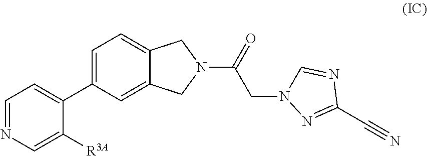
or a pharmaceutically acceptable salt thereof.

10. The compound of claim 7, or a pharmaceutically acceptable salt thereof wherein R^{3A} is independently selected from -Me, —OH, —F, —Cl, —CH₂F, —CHF₂, —CF₃, —CH₂CF₃, —OMe, —OCF₃ and —O—CH₂—CF₃.

11. The compound of claim 1 selected from:
1-(2-(5-(2-(difluoromethyl)pyridin-3-yl)isoindolin-2-yl)-2-oxoethyl)-1H-1,2,4-triazole-3-carbonitrile; 1-(2-(5-(3-chloro-5-(trifluoromethyl)pyridin-4-yl)isoindolin-2-yl)-2-oxoethyl)-1H-1,2,4-triazole-3-carbonitrile; 1-(2-(5-(5-chloro-2-fluoropyridin-4-yl)isoindolin-2-yl)-2-oxoethyl)-1H-1,2,4-triazole-3-carbonitrile; 1-(2-(5-(3-chloro-2-(trifluoromethyl)phenyl)isoindolin-2-yl)-2-oxoethyl)-1H-1,2,4-triazole-3-carbonitrile; 1-(2-oxo-2-(5-(2-(2,2,2-trifluoroethoxy)pyridin-3-yl)isoindolin-2-yl)ethyl)-1H-1,2,4-triazole-3-carbonitrile; 1-(2-(5-(6-methyl-2-(trifluoromethyl)pyridin-3-yl)isoindolin-2-yl)-2-oxoethyl)-1H-1,2,4-triazole-3-carbonitrile; 1-(2-(5-(5-fluoro-2-(trifluoromethyl)pyridin-3-yl)isoindolin-2-yl)-2-oxoethyl)-1H-1,2,4-triazole-3-carbonitrile; 1-(2-(5-(5-fluoro-4-(trifluoromethyl)pyridin-3-yl)isoindolin-2-yl)-2-oxoethyl)-1H-1,2,4-triazole-3-carbonitrile; 1-(2-(5-(2-chloro-4,6-difluorophenyl)isoindolin-2-yl)-2-oxoethyl)-1H-1,2,4-triazole-3-carbonitrile; 1-(2-(5-(4-fluoro-2-(pyrrolidin-1-yl)phenyl)isoindolin-2-yl)-2-oxoethyl)-1H-1,2,4-triazole-3-carbonitrile; 1-(2-(5-(2-fluoro-6-(trifluoromethyl)phenyl)isoindolin-2-yl)-2-oxoethyl)-1H-1,2,4-triazole-3-carbonitrile; 1-(2-(5-(2-methylpyridin-3-yl)isoindolin-2-yl)-2-oxoethyl)-1H-1,2,4-triazole-3-carbonitrile; 1-(2-(5-(6-methoxy-2-(trifluoromethyl)pyridin-3-yl)isoindolin-2-yl)-2-oxoethyl)-1H-1,2,4-triazole-3-carbonitrile; 1-(2-(5-(2-chloro-3,6-difluorophenyl)isoindolin-2-yl)-2-oxoethyl)-1H-1,2,4-triazole-3-carbonitrile; 1-(2-(5-(3-fluoro-5-(trifluoromethyl)pyridin-4-yl)isoindolin-2-yl)-2-oxoethyl)-1H-1,2,4-triazole-3-carbonitrile; 1-(2-oxo-2-(5-(2-(trifluoromethoxy)pyridin-3-yl)isoindolin-2-yl)ethyl)-1H-1,2,4-triazole-3-carbonitrile; 1-(2-(5-(4-chloro-2-(trifluoromethyl)pyridin-3-yl)isoindolin-2-yl)-2-oxoethyl)-1H-1,2,4-triazole-3-carbonitrile; 1-(2-oxo-2-(5-(2-(2,2,2-trifluoroethyl)pyridin-3-yl)isoindolin-2-yl)ethyl)-1H-1,2,4-triazole-3-carbonitrile; 1-(2-(5-(4-fluoro-2-(trifluoromethyl)pyridin-3-yl)isoindolin-2-yl)-2-oxoethyl)-1H-1,2,4-triazole-3-carbonitrile; 1-(2-(5-(2-chloro-4-fluoropyridin-3-yl)isoindolin-2-yl)-2-oxoethyl)-1H-1,2,4-triazole-3-carbonitrile; 1-(2-(5-(2-fluoro-4-(trifluoromethyl)pyridin-3-yl)isoindolin-2-yl)-2-oxoethyl)-1H-1,2,4-triazole-3-carbonitrile; 1-(2-(5-(3-chloro-5-fluoropyridin-4-yl)isoindolin-2-yl)-2-oxoethyl)-1H-1,2,4-triazole-3-carbonitrile; 1-(2-(5-(4-chloro-2-fluoropyridin-3-yl)isoindolin-2-yl)-2-oxoethyl)-1H-1,2,4-triazole-3-carbonitrile; 1-(2-(5-(1-methyl-3-(trifluoromethyl)-1H-pyrazol-4-yl)isoindolin-2-yl)-2-oxoethyl)-1H-1,2,4-triazole-3-carbonitrile; 1-(2-(5-(6-fluoro-2-(trifluoromethyl)pyridin-3-yl)isoindolin-2-yl)-2-oxoethyl)-1H-1,2,4-triazole-3-carbonitrile; 1-(2-oxo-2-(5-(3-(trifluoromethyl)pyridin-4-yl)isoindolin-2-yl)ethyl)-1H-1,2,4-triazole-3-carbonitrile; 1-(2-(5-(5-fluoro-2-(trifluoromethyl)phenyl)isoindolin-2-yl)-2-oxoethyl)-1H-1,2,4-triazole-3-carbonitrile; 1-(2-(5-(4-fluoro-2-(trifluoromethyl)phenyl)isoindolin-2-yl)-2-oxoethyl)-1H-1,2,4-triazole-3-carbonitrile; 1-(2-(5-(2-chloro-4,5-difluorophenyl)isoindolin-2-yl)-2-oxoethyl)-1H-1,2,4-triazole-3-carbonitrile; 1-(2-(5-(4-methoxy-2-(trifluoromethyl)phenyl)isoindolin-2-yl)-2-oxoethyl)-1H-1,2,4-triazole-3-carbonitrile; 1-(2-(5-(2-cyano-4-fluorophenyl)isoindolin-2-yl)-2-oxoethyl)-1H-1,2,4-triazole-3-carbonitrile; 1-(2-(5-(2-chlorophenyl)isoindolin-2-yl)-2-oxoethyl)-1H-1,2,4-triazole-3-carbonitrile; 1-(2-(5-(2-chloro-4-methoxyphenyl)isoindolin-2-yl)-2-oxoethyl)-1H-1,2,4-triazole-3-carbonitrile; 1-(2-(5-(2,6-dichlorophenyl)isoindolin-2-yl)-2-oxoethyl)-1H-1,2,4-triazole-3-carbonitrile; 1-(2-oxo-2-(5-(2-(trifluoromethyl)phenyl)isoindolin-2-yl)ethyl)-1H-1,2,4-triazole-3-carbonitrile; 1-(2-(5-(2-chloro-4-fluorophenyl)isoindolin-2-yl)-2-oxoethyl)-1H-1,2,4-triazole-3-carbonitrile; 1-(2-(5-(2,4-dichlorophenyl)isoindolin-2-yl)-2-oxoethyl)-1H-1,2,4-triazole-3-carbonitrile; 1-(2-(5-(4-fluoro-2-(2-methoxyethoxy)phenyl)isoindolin-2-yl)-2-oxoethyl)-1H-1,2,4-triazole-3-carbonitrile; 1-(2-oxo-2-(5-(2-(trifluoromethoxy)phenyl)isoindolin-2-yl)ethyl)-1H-1,2,4-triazole-3-carbonitrile; 1-(2-(5-(5-cyano-2-fluorophenyl)isoindolin-2-yl)-2-oxoethyl)-1H-1,2,4-triazole-3-carbonitrile; 1-(2-(5-(2-chloro-4-(trifluoromethyl)phenyl)isoindolin-2-yl)-2-oxoethyl)-1H-1,2,4-triazole-3-carbonitrile; 1-(2-(5-(2-cyanophenyl)isoindolin-2-yl)-2-oxoethyl)-1H-1,2,4-triazole-3-carbonitrile; 1-(2-(5-(3-cyano-2-fluorophenyl)isoindolin-2-yl)-2-oxoethyl)-1H-1,2,4-triazole-3-carbonitrile; 1-(2-oxo-2-(5-(2,4,5-trifluorophenyl)isoindolin-2-yl)ethyl)-1H-1,2,4-triazole-3-carbonitrile; 1-(2-(5-(4-cyanophenyl)isoindolin-2-yl)-2-oxoethyl)-1H-1,2,4-triazole-3-carbonitrile; 1-(2-(5-(2-chloropyridin-3-yl)isoindolin-2-yl)-2-oxoethyl)-1H-1,2,4-triazole-3-carbonitrile; 1-(2-(5-(1-methyl-3-(trifluoromethyl)-1H-pyrazol-5-yl)isoindolin-2-yl)-2-oxoethyl)-1H-1,2,4-triazole-3-carbonitrile; 1-(2-(5-(3-chloropyridin-4-yl)isoindolin-2-yl)-2-oxoethyl)-1H-1,2,4-triazole-3-carbonitrile; 1-(2-(5-(2-acetylphenyl)isoindolin-2-yl)-2-oxoethyl)-1H-1,2,4-triazole-3-carbonitrile; 1-(2-(5-(4-fluoro-2-methoxyphenyl)isoindolin-2-yl)-2-oxoethyl)-1H-1,2,4-triazole-3-carbonitrile; 1-(2-(5-(5-chloro-2-methoxyphenyl)isoindolin-2-yl)-2-oxoethyl)-1H-1,2,4-triazole-3-carbonitrile; 1-(2-(5-(2,4-dimethoxyphenyl)isoindolin-2-yl)-2-oxoethyl)-1H-1,2,4-triazole-3-carbonitrile; 1-(2-(5-(3-cyanophenyl)isoindolin-2-yl)-2-oxoethyl)-1H-1,2,4-triazole-3-carbonitrile; 1-(2-(5-(2,4-difluorophenyl)isoindolin-2-yl)-2-oxoethyl)-1H-1,2,4-triazole-3-carbonitrile; 1-(2-(5-(6-fluoropyridin-3-yl)isoindolin-2-yl)-2-oxoethyl)-1H-1,2,4-triazole-3-carbonitrile; 1-(2-(5-(5-fluoro-2-methoxyphenyl)isoindolin-2-yl)-2-oxoethyl)-1H-1,2,4-triazole-3-carbonitrile; 1-(2-(5-(4-chloro-2-fluorophenyl)isoindolin-2-yl)-2-oxoethyl)-1H-1,2,4-triazole-3-carbonitrile; 1-(2-(5-(2,5-dimethoxyphenyl)isoindolin-2-yl)-2-oxoethyl)-1H-1,2,4-triazole-3-carbonitrile; 1-(2-(5-(3-fluoropyridin-4-yl)isoindolin-2-yl)-2-oxoethyl)-1H-1,2,4-triazole-3-carbonitrile; 1-(2-(5-(4-methoxyphenyl)isoindolin-2-yl)-2-oxoethyl)-1H-1,2,4-triazole-3-carbonitrile; 1-(2-(5-(2-methoxy-5-(trifluoromethoxy)phenyl)isoindolin-2-yl)-2-oxoethyl)-1H-1,2,4-triazole-3-carbonitrile; 1-(2-(5-(2-fluoropyridin-4-yl)isoindolin-2-yl)-2-oxoethyl)-1H-1,2,4-triazole-3-carbonitrile; 1-(2-(5-(4-ethynylphenyl)isoindolin-2-yl)-2-oxoethyl)-1H-1,2,4-triazole-3-carbonitrile; 1-(2-(5-(2-fluorophenyl)isoindolin-2-yl)-2-oxoethyl)-1H-1,2,4-triazole-3-carbonitrile; 1-(2-oxo-2-(5-(pyridin-4-yl)isoindolin-2-yl)ethyl)-1H-1,2,4-triazole-3-carbonitrile; 1-(2-(5-(4-bromophenyl)isoindolin-2-yl)-2-oxoethyl)-1H-1,2,4-triazole-3-carbonitrile; 1-(2-(5-(3-chloro-4-fluorophenyl)isoindolin-2-yl)-2-oxoethyl)-1H-1,2,4-triazole-3-carbonitrile; 1-(2-(5-([1,1′-biphenyl]-2-yl)isoindolin-2-yl)-2-oxoethyl)-1H-1,2,4-triazole-3-carbonitrile; 1-(2-(5-(4-chloro-3-fluorophenyl)isoindolin-2-yl)-2-oxoethyl)-1H-1,2,4-triazole-3-carbonitrile; 1-(2-(5-(4-chlorophenyl)isoindolin-2-yl)-2-oxoethyl)-1H-1,2,4-triazole-3-carbonitrile; 1-(2-(5-(3,4-difluorophenyl)isoindolin-2-yl)-2-oxoethyl)-1H-1,2,4-triazole-3-carbonitrile; 1-(2-(5-(2-fluoropyridin-3-yl)isoindolin-2-yl)-2-oxoethyl)-1H-1,2,4-triazole-3-carbonitrile; 1-(2-(5-(3,5-difluorophenyl)isoindolin-2-yl)-2-oxoethyl)-1H-1,2,4-triazole-3-carbonitrile; 1-(2-(5-(2-methoxyphenyl)isoindolin-2-yl)-2-oxoethyl)-1H-1,2,4-triazole-3-carbonitrile; 1-(2-oxo-2-(5-(4-(trifluoromethyl)phenyl)isoindolin-2-yl)ethyl)-1H-1,2,4-triazole-3-carbonitrile; 1-(2-(5-(3-methoxyphenyl)isoindolin-2-yl)-2-oxoethyl)-1H-1,2,4-triazole-3-carbonitrile; 1-(2-(5-(3-methyl-4-(trifluoromethyl)phenyl)isoindolin-2-yl)-2-oxoethyl)-1H-1,2,4-triazole-3-carbonitrile; 1-(2-oxo-2-(5-phenylisoindolin-2-yl)ethyl)-1H-1,2,4-triazole-3-carbonitrile; 1-(2-(5-(3-chlorophenyl)isoindolin-2-yl)-2-oxoethyl)-1H-1,2,4-triazole-3-carbonitrile; 1-(2-(5-(3-fluorophenyl)isoindolin-2-yl)-2-oxoethyl)-1H-1,2,4-triazole-3-carbonitrile; 1-(2-(5-(4-fluorophenyl)isoindolin-2-yl)-2-oxoethyl)-1H-1,2,4-triazole-3-carbonitrile; 1-(2-oxo-2-(5-(4-(trifluoromethoxy)phenyl)isoindolin-2-yl)ethyl)-1H-1,2,4-triazole-3-carbonitrile; 1-(2-(5-(3-fluoro-2-(trifluoromethyl)phenyl)isoindolin-2-yl)-2-oxoethyl)-1H-1,2,4-triazole-3-carbonitrile; 1-(2-oxo-2-(5-(2-(trifluoromethyl)pyridin-3-yl)isoindolin-2-yl)ethyl)-1H-1,2,4-triazole-3-carbonitrile; 1-(2-oxo-2-(5-(4-(trifluoromethyl)pyridin-3-yl)isoindolin-2-yl)ethyl)-1H-1,2,4-triazole-3-carbonitrile.

12. A pharmaceutical composition comprising at least one of the compounds of claim 1 or a pharmaceutically acceptable salt thereof, and at least one pharmaceutically acceptable carrier, diluent or excipient.

13. The pharmaceutical composition of claim 12 further comprising additional therapeutic agent(s).

14. A compound of claim 1 for use in therapy, alone, or optionally in combination with another compound of claim 1, and/or at least one other type of therapeutic agent.

15. A compound of claim 1 for use in the treatment of the pathology and/or symptomology of a disease caused by a parasite, alone, or optionally in combination with another compound of claim 1 and/or at least one other type of therapeutic agent.

16. A method for the treatment of the pathology and/or symptomology of a disease caused by a parasite, comprising administering to a patient in need of such treatment a therapeutically effective amount of at least one of the compounds of claim 1, alone, or optionally in combination with another compound of claim 1, and/or at least one other type of therapeutic agent.

17. A method for the treatment of the pathology and/or symptomology of a disease caused by a parasite, comprising administering to a patient in need thereof a therapeutically effective amount of a first and second therapeutic agent, wherein the first therapeutic agent is a compound of claim 1 and the second therapeutic agent is one other type of therapeutic agent.

18. Use of a compound claim 1 for the manufacture of a medicament for the treatment of the pathology and/or symptomology of a disease caused by a parasite, alone, or optionally in combination with another compound of the present invention and/or at least one other type of therapeutic agent.

19. A combined preparation of a compound of claim 1 and additional therapeutic agent(s) for simultaneous, separate or sequential use in therapy.

20. A combined preparation for use in the treatment of the pathology and/or symptomology of a disease caused by a parasite comprising a compound of claim 1 and an additional therapeutic agent(s) for simultaneous, separate or sequential use in the treatment of the pathology and/or symptomology of a disease caused by a parasite.

21.-22. (canceled)
